(19) United States
(12) Patent Application Publication
(10) Pub. No.: US 2024/0093233 A1
(43) Pub. Date: Mar. 21, 2024

(54) MEASLES-HIV OR MEASLES-HTLV VACCINE

(71) Applicants: VIROXIS, PARIS (FR); INSTITUT PASTEUR, PARIS CEDEX 15 (FR); CENTRE NATIONAL DE LA RECHERCHE SCIENTIFIQUE, PARIS CEDEX 16 (FR); INSTITUT GUSTAVE ROUSSY (IGR), VILLEJUIF (FR)

(72) Inventors: Thierry HEIDMANN, VILLEJUIF CEDEX (FR); Frederic TANGY, PARIS CEDEX 15 (FR)

(21) Appl. No.: 18/261,051

(22) PCT Filed: Jan. 13, 2022
(86) PCT No.: PCT/EP2022/050693

§ 371 Date: Jul. 11, 2023

(30) Foreign Application Priority Data
Jan. 13, 2021 (EP) 21305032.1
Jan. 13, 2021 (EP) 21305033.9

(51) Int. Cl. C12N 15/86; A61P 31/18; C07K 14/15; C12N 7/00

(52) U.S. Cl. CPC C12N 15/86; A61P 31/18; C07K 14/15; C12N 7/00; C12N 2740/16122; C12N 2760/18422; C12N 2760/18443; C12N 2770/24122

(57) ABSTRACT

The invention relates to recombinant measles virus expressing Immunodeficiency virus (IV) or HTLV polypeptides, and concerns in particular immunogenic immunodeficiency virus particles expressed by a measles virus and/or virus like particles (VLPs) that contain proteins of at least one immunodeficiency virus or Human T-lymphotropic virus. These particles may be recombinant infectious particles able to replicate in a host after an administration. The invention provides means, in particular nucleic acid constructs, vectors, cells and rescue systems to produce these recombinant infectious particles. The invention also relates to the use of these recombinant infectious particles, in particular under the form of a composition, more particularly in a vaccine formulation, for the treatment or prevention of an infection by HIV or HTLV.

### FIELD OF THE INVENTION

The application generally relates to recombinant genetic constructs comprising a recombinant measles virus cDNA comprising heterologous polynucleotides encoding immunodeficiency virus (IV) antigens or Human T-Lymphotropic Virus (HTLV) antigens, including protein, polypeptide, antigenic fragment thereof and mutated version thereof, in particular at least one Human (HIV; in particular HIV-1 or HIV-2), Simian (SIV) or Feline (FIV) (all three referenced under the acronym IV in the present description) immunodeficiency virus, or HTLV-1, HTLV-2 or HTLV-3 virus antigen, protein, polypeptide, antigenic fragment thereof and mutated version thereof. The application also relates to the uses of genetic constructs or viruses, and more particularly their applications for inducing protection against the immunodeficiency virus or the HTLV, and/or the measles virus (MV or MeV).

The means of the invention are more particularly dedicated to a combination of recombinant nucleic acid constructs allowing the expression of at least one of the following antigens of a determined IV or HTLV, or a truncated version thereof, or a mutated version thereof or an antigenic fragment thereof: GAG, ENV and NEF, wherein the polynucleotides encoding these polypeptides may be issued or derived from the HIV, the SIV and the FIV, in particular HIV from any known clade, and more particularly HIV-1 or HIV-2, and most preferably from HIV-1, and wherein the immunosuppressive domain of ENV and/or NEF is mutated. The means of the invention are more particularly dedicated to a combination of recombinant nucleic acid constructs allowing the expression of at least one of the following antigens of a determined HTLV, or a truncated version thereof, or a mutated version thereof or an antigenic fragment thereof: GAG, ENV and HBZ (and TAX when applicable), in particular HBZ and TAX when applicable, wherein the polynucleotides encoding these polypeptides may be issued or derived from an HTLV, in particular HTLV-1, HTLV-2 or HTLV-3, and more particularly HTLV-1, and wherein the immunosuppressive domain of ENV is mutated.

The invention also relates to a recombinant MeV-IV or MeV-HTLV virus expressing at least one of the previously mentioned IV or HTLV antigen, polypeptide, antigenic fragment thereof or mutated version thereof, namely GAG and ENV, and NEF or HBZ (and TAX) when applicable. The invention also concerns immunogenic viral particles, like Virus Like Particles, expressed by the recombinant measles virus and comprising IV or HTLV antigens, in particular at least the GAG and ENV, and NEF or HBZ (and TAX) when applicable, antigens, polypeptides, antigenic fragments thereof, truncated and/or mutated versions thereof, and/or infectious Virus-like particles (VLPs) that contains at least the GAG, ENV, and possibly NEF or HBZ (and TAX) polypeptides, or proteins, or antigenic fragments thereof, or mutated versions thereof, said immunogenic particles and/or VLPs being able to elicit a cellular and/or humoral response against IV or HTLV, in particular a IFNγ and/or IL-2 response.

In particular, the invention is related to the use of these genetic constructs, recombinant nucleic acid constructs, expression vectors like plasmid vectors and the like, recombinant virus infectious particles, VLPs, for inducing an immunogenic response within a host against a HIV, SIV or HTLV infection, and more particularly against a HIV-1, HIV-2, HTLV-1, HTLV-2 or HTLV-3 infection.

### BACKGROUND OF THE INVENTION

The human immunodeficiency virus (HIV) is the causative agent of one of the most dangerous human diseases, the acquired immune deficiency syndrome (AIDS). HIV is a member of the genus Lentivirus, which is a member of the Retroviridae family. Lentiviruses are single-stranded, positive sense, enveloped RNA viruses. Upon entry into the target cell, the viral RNA genome is reverse transcribed into a double-stranded DNA by a virally encoded reverse transcriptase. HIV virus is composed of two copies of positive-sense single-stranded RNA which codes for the virus's nine genes. The virus genome is enclosed by a capsid composed of viral protein p24. A matrix composed of the viral protein p17 surrounds the capsid, ensuring the integrity of the virion particle. The single-stranded RNA is bound to the nucleocapsid protein p7. The RNA genome consists of structural landmarks and nine genes (gag, pol, env, tat, rev, nef, vif, vpr and vpu), encoding 19 proteins.

Since the discovery of HIV more than 30 years ago, various strategies have been developed to prevent the disease. As an example, antiviral drugs have been developed to block various stages of the virus life cycle. Such an approach has allowed the suppression of the amplification of the virus in its host, and has lowered the integration of the virus into the genomic material of the infected cells of its host; these results significantly prolong the life of HIV-infected patients. Nonetheless, the overall suppression of the virus within the host has not been reached yet. Moreover, viral resistance to many anti-HIV drugs has been seen, leading to the spreading of HIV-drug resistant viral forms. Due to the highly variable nature of HIV, because the HIV reverse-transcriptase lacks proofreading capability, drug-resistant forms of HIV constantly emerge in HIV patients. There is therefore a need for a treatment which would effectively suppress HIV. The development of a vaccine candidate for preventing or treating the disease is one of the most promising strategy to effectively prevent HIV-infection or block HIV-replication and/or integration in a patient. The most promising strategy is to provide a vaccine which prevents infection by a HIV; such a vaccine could also elicit a therapeutic response within a human being, thereby treating a host already infected by a HIV.

One of the most promising therapy for preventing HIV infections is prophylactic vaccination but no such vaccine is currently available. Prophylaxis would be the easiest and safest way to control the HIV infections, and protect the populations against infection with HIV. In this context, the development of a preventive treatment, like a preventive vaccine, is a major priority to meet the needs of human population. There is therefore a need for a fully efficient treatment able to treat or prevent HIV infections, including to prevent outcomes of HIV primary infection, in particular to prevent the HIV infection and to prevent the apparition of acquired immunodeficiency syndrome.

Several vaccine candidates have been developed in the recent years. Unfortunately, among the six clinical efficacy trials performed to date with HIV vaccine candidates, only the RV144 trial has showed some level of protection with an estimated efficacy of 31% at 42 months, pointing out the importance of the vector/antigen combination for an HIV vaccine (2). Indeed, administration of HIV-canarypox vector (ALVAC HIV) prime followed by boosts with gp120 Env protein (AIDS VAX B/E) may have induced short-term immunity as revealed by the decrease of vaccine efficacy over the first year and the high viral load and the CD4 T cell depletion observed in vaccinated individuals who became infected in the RV144 trial (2, 3).

Vaccination with replication incompetent Adenovirus 5 (Ad5) expressing HIV antigens had no efficacy, and even increased the sensitivity to infection likely because of pre-existing anti-Ad5 antibody immunity in volunteers (4). Other clinical studies have also emphasized the limitation of vaccination using either only HIV T-cell epitopes (Step study, 2) or only envelope protein (Vax003, Vax004, 5, 6).

Human T-cell Leukemia Virus type-1 (HTLV-1) was first described early in the 80's, before discovery of Human Immunodeficiency Virus type-1 (HIV-1). Both are retroviruses that emerged in human populations after zoonotic transmission from simian populations.

It is estimated that approximately 10 million people are HTLV-1-infected, in comparison with 37 million people being HIV-infected worldwide. Both HTLV-1 and HIV-1 lead to chronic infection. HTLV-1 infection may lead to the development of two main diseases: a malignant lymphoproliferation named Adult T-cell Leukemia/Lymphoma (ATLL), and a chronic progressive myelopathy named Tropical Spastic Para paresis/HTLV-1 Associated Myelopathy (TSP/HAM). Approximately 2 to 4% of HTLV-1 infected individuals will develop an ATLL, while between 1 and 2% will develop TSP/HAM. In vivo, HTLV-1 spread occurs through two mechanisms: neo-infection or clonal expansion of infected cells. HTLV-1 full-length integrated viral genome is mainly found in activated CD4+ T-cells; proviral DNA is also detected, but to a lesser extent, in CD8+ T-cells, B cells, monocytes and dendritic cells.

HTLV-1 has a monopartite, linear, dimeric single strand RNA (+) genome of 8,5 kb, with a 5′-cap and a 3′ poly-A tail. There are two long terminal repeats (LTRs) of about 600 nucleotide residues at the 5′ and 3′ ends. The LTRs contain the U3, R and U5 regions. There are also a primer binding site (PBS) at the 5′ end and a polypurin tract (PPT) at the 3′ end. The integrated virus uses the promoter elements in the 5′LTR to initiate and drive transcription, giving rise to the unspliced full length mRNA that will serve as genomic RNA to be packaged into virions. The genomic RNA of HTLV-1 encodes the structural and enzymatic proteins GAG, ENV and POL, which are similar to other retroviruses. HTLV-1 differs from other retrovirus by a unique region towards the 3′ end, designated the pX region, which encodes regulatory proteins such as TAX and REX and additional proteins like HBZ (basic leucine zipper (bZIP) factor (HBZ) encoded in reverse orientation) whose functions are now well documented (46).

There is no vaccine to prevent HTLV-1 infection or HTLV-1 related disease. The feasibility of an anti-HTVL-1 vaccine has been supported first by the worldwide genetic stability among HTLV-1 strains, secondly by promising results after vaccination in animal models, and finally by the presence of a potent HTLV-1-associated immune response in infected individuals. Despites these interesting assets, the need for an anti-HTVL-1 vaccine is not fulfilled.

Therefore, there is a need for a vaccine and products such as active ingredients for preparing a vaccine, and method for producing these products and vaccine. The vaccine candidate should be safe and efficient when immunizing people in need thereof, without significant side effects, and induce the production of antibodies neutralizing the HIV or the HTLV-1, and possibly T cells including T helper cells and/or Cytotoxic T cells. In other words, the vaccine should elicit a strong cellular and/or humoral response. Advantageously, the vaccine should confer sterilizing immunity after a single immunization or a prime-boost immunization. To this end, there is a need for a vaccine that would enable the HIV proteins and/or HIV VLPs or the HTLV proteins and/or HTLV VLPs to be generated in vivo, in particular in infected cells of a host, and thus provide an efficient, long-lasting immunity, especially which induces life-long immunity after only a single, or two or several administration steps within a homologous or heterologous administration regimen (e.g. homologous prime-boost or heterologous prime-boost administration regimen).

Another need is to facilitate the vaccination of the populations that hardly have access to medical centers or the like. A vaccine candidate that would elicit immunization against multiple disease agents could enhance global health of these populations. A single vaccination could therefore allow the immunization against several disease agents present in geographical regions of interest. In particular, with the aim to totally eradicate the measles virus (MeV), a vaccine immunizing against both the MeV and the HIV or the HTLV could clearly protect these populations against these two major threats.

Live attenuated measles vaccine has been safely administered to over a billion children during the last 40 years, affording life-long protection with an efficacy rate of 93-97% after one or two administrations. MV vectors are immunogenic in mice and NHP (Non-Human Primates) inducing long-term neutralizing antibodies and cellular immunity, even in presence of pre-immunity to the vector, and preclinical protection from lethal challenges has been shown for numerous pathogens (7). Protective immunity against a heterologous pathogenic agent using MeV as a delivery vector relies on in vivo replication of MeV resulting in the expression of heterologous antigens in vivo in immune cells naturally targeted by measles virus. The proof of concept of this technology in humans has been demonstrated for a measles chikungunya vaccine (MV-CHIK) that was successfully tested in clinical trial (8). The vaccine was well tolerated and induced a robust and functional antibody response in 100% of volunteers after 2 immunizations. Most importantly, pre-existing measles antibodies did not impair the immunogenicity of the heterologous antigen, confirming that pre-immunity to measles due to vaccination or infection does not restrict the use of recombinant MV for new vaccines (8).

Measles virus has been isolated in 1954 (9). Measles virus is a member of the order mononegavirales, i.e. viruses with a non-segmented negative-strand RNA genome. The non-segmented genome of MeV has an antimessage polarity which results in a genomic RNA which is neither translated in vivo or in vitro nor infectious when purified. Transcription and replication of non-segmented (−) strand RNA viruses and their assembly into virus particles have been studied and reported especially in Fields virology (10). Transcription and replication of the measles virus do not involve the nucleus of the infected cells but rather take place in the cytoplasm of host cell. The genome of the MeV comprises genes encoding six major structural proteins designated N, P, M, F, H and L, and an additional two non-structural proteins from the P gene, designated C and V. The gene order is the following: from the 3′ end of the genomic RNA; N, P (including C and V), M, F, H and L large polymerase at the 5′ end. The genome furthermore comprises non coding regions in the intergenic region M/F. This non coding region contains approximatively 100 nucleotides of untranslated RNA. The cited genes of MeV respectively encode the proteins of the nucleocapsid of the virus or nucleoprotein (N), the phosphoprotein (P), the large protein (L) which together assemble around the genome RNA to provide the nucleocapsid, the hemagglutinin (H), the fusion protein (F) and the matrix protein (M).

Attenuated viruses have been derived from MeV virus to provide vaccine strains, in particular from the Schwarz strain or strains derived therefrom. The Schwarz measles vaccine is a safe and efficient vaccine currently available for preventing measles. Besides providing vaccine, strains attenuated measles virus such as the Schwarz strain have shown to be stable and suitable for the design of efficient delivery vector for immunization against other viruses, like Zika virus or Chikungunya virus (11).

### SUMMARY OF THE INVENTION

To address, at least partially, the drawbacks of the state of the art, the inventors achieved the production of active components (or ingredients) for vaccines based on recombinant genetic constructs, and especially based on recombinant nucleic acid constructs comprising, within an infectious replicative measles virus, cloned antigen(s) or polynucleotide(s), or mutated version(s) thereof, encoding immunodeficiency virus (IV) polypeptides, proteins or antigens, or antigenic fragments thereof, or encoding Human T-cell Leukemia Virus type-1 (HTLV-1) polypeptides, proteins or antigens, or antigenic fragments thereof. Vaccines may be recovered when the recombinant measles virus replicates in the host after administration. The invention thus relates to a IV vaccine or to a HTLV-1 vaccine, especially a pediatric vaccine, and relates to active ingredient based on an attenuated measles virus strain such as a known vaccine strain commercially available, especially the widely used Schwarz measles vaccine. For all these reasons, the inventors used attenuated measles viruses to generate recombinant measles virus particles stably expressing polypeptides of IV or HTLV, in particular immunogenic virus particles thereof and/or VLPs. The measles approach of the invention meets all of the relevant criteria of a future IV or HTLV vaccine, in particular for a future HIV or HTLV vaccine.

One aim of the invention is to provide a genetic construct, in particular recombinant genetic constructs, in particular recombinant nucleic acid constructs, suitable to recover Measles virus expressing IV antigens or particles or HTLV antigens or particles, and optionally also IV Virus Like Particles (IV-VLPs) or HTLV Virus Like Particles (HTLV-VLPs).

In the present invention, MeV-SHIV (Simian Human Immunodeficiency Virus; i.e. a MeV construct comprising both antigens issued from a Simian IV and from a Human IV) vectors expressing simultaneously Gag-Env to form virus-like-particles (VLPs) were generated. The sequences corresponding to SIV239 gag and HIV-1 env genes were inserted into two distinct additional transcription units (ATU) (consensus B Env for prime and SF162 Env for boosts). Another MV vector was generated expressing SIV239 Nef under a secreted and non-myristoylated form. Specific vectors were also generated with targeted mutations within the HIV Env and SIV Nef immunosuppressive domains (also referenced IS or ISD in the present description). Indeed, HIV possesses not only an IS domain within its Env, but also within the Nef protein (13, 14). In the present invention, MeV-HTLV (i.e. a MeV construct comprising antigens issued from HTLV) vectors expressing simultaneously Gag-Env to form virus-like-particles (VLPs) that were previously demonstrated to be very immunogenic were generated. The sequences corresponding to gag and env genes of HTLV-1 were inserted into two distinct additional transcription units (ATU). Specific vectors were also generated with targeted mutations within the Env immunosuppressive domains (also referenced IS or ISD in the present description). Indeed, HTLV possesses an IS domain within its Env protein. An antigen having lost, or substantially lost, its immunosuppressive function may elicit an efficient immune response. This enables the individuals once infected by the virus to allow the immune system to destroy the infected cells and prevent/cure the infection. Mutations within the immunosuppressive domain of ENV and/or NEF abolish the immunosuppressive properties of these proteins. Mutations of immunosuppressive domains have been shown to restore tumor cells sensitivity to immune-rejection (15, 16) and to improve vaccine-immunity (17). ENV protein is known to confer an immunosuppressive function to virus expressing such a protein. An adequately mutated immunosuppressive domain lowers the immunosuppressive function of the mutated protein (or antigen), while having a limited impact on the structure of the protein, thereby allowing normal expression and conformation (i.e. folding) of the protein. A virus expressing a protein mutated within its ISD may therefore be less immunosuppressive than its wild-type counterparts, while its other functions are not impaired. As an example, a ENV protein with a mutated ISD lowers the immunosuppressive effect of a virus expressing such ENV protein, but the envelope function of ENV is not impacted by the mutation, leading to the expression of a virus with ENV protein sharing the same structure as wild-type ENV protein. The immunosuppressive property of a given protein can be measured by following the general procedure described in Mangeney & Heidmann (18) and Mangeney et al. (19).

Mutated ENV and NEF proteins of HIV and ENV of HTLV have been disclosed respectively in U.S. patent application Ser. No. 14/363,095, International application publication No. WO2005095442 (HIV NEF), and International application publication No. WO2013083799 (HIV NEF) and International Patent publication No. WO2005095441 (HTLV ENV), wherein several mutations within the ISD of ENV or NEF are disclosed respectively, illustrating which mutations allow the expression of more immunogenic viruses expressing a mutated ENV and/or NEF.

According to a first aspect, the invention concerns a nucleic acid construct which comprises a cDNA molecule encoding a full length antigenomic (+) RNA strand of a measles virus (MeV); and
(i) a first heterologous polynucleotide encoding at least one GAG antigen, a fragment thereof, or a mutated version thereof of a Simian Immunodeficiency Virus (SIV), a Human Immunodeficiency Virus (HIV), or a Human T-lymphotropic virus (HTLV), wherein the first heterologous polynucleotide is operatively cloned within an additional transcription unit (ATU) inserted within the cDNA of the antigenomic (+) RNA, in particular an ATU located between the P gene and the M gene of the MeV, in particular in the ATU2 inserted between the P gene and the M gene of the MeV;
(ii) a second heterologous polynucleotide encoding at least one ENV antigen, or a fragment thereof comprising an immunosuppressive domain (ISD), in particular at least one fragment comprising the transmembrane subunit of the ENV antigen, wherein the ENV antigen or its fragment is mutated within its immunosuppressive domain (ISD) and is of a Simian Immunodeficiency Virus (SIV), a Human Immunodeficiency Virus (HIV) or a Human T-lymphotropic virus (HTLV), wherein the second heterologous polynucleotide is operatively cloned within the same or a different additional transcription unit (ATU) as in (i) inserted within the cDNA of the antigenomic (+) RNA, in particular an ATU located between the H gene and the L gene of the MeV, in particular in the ATU3 inserted between the H gene and the L gene of the MeV;
wherein the mutation within the ISD domain of ENV reduces the immunosuppressive index of the ENV antigen; and wherein the GAG and ENV antigens, or their respective immunogenic fragments or mutated versions thereof, all originate from the same virus type, in particular are from the same virus strain, more particularly from HIV or HTLV, and preferably from HIV-1 or HIV-2 or HTLV-1.

In a second aspect, the invention concerns a nucleic acid construct which comprises a cDNA molecule encoding a full length antigenomic (+) RNA strand of a measles virus (MeV); and
(i) a first heterologous polynucleotide encoding at least one GAG antigen, a fragment thereof, or a mutated version thereof of a Simian Immunodeficiency Virus (SIV), a Human Immunodeficiency Virus (HIV), or a Human T-lymphotropic virus (HTLV), wherein the first heterologous antigen is operatively cloned within an additional transcription unit (ATU) inserted within the cDNA of the antigenomic (+) RNA, in particular an ATU located between the P gene and the M gene of the MeV, in particular in the ATU2 inserted between the P gene and the M gene of the MeV;
(ii) a second heterologous polynucleotide encoding at least one ENV antigen, or a fragment thereof, comprising an immunosuppressive domain (ISD), in particular at least one fragment comprising the transmembrane subunit of the ENV antigen, wherein the ENV antigen or its fragment is mutated within its immunosuppressive domain (ISD) and is of a Simian Immunodeficiency Virus (SIV), a Human Immunodeficiency Virus (HIV) or a Human T-lymphotropic virus (HTLV), wherein the second heterologous antigen is operatively cloned within the same or an additional transcription unit (ATU) as in (i) inserted within the cDNA of the antigenomic (+) RNA, in particular an ATU located between the H gene and the L gene of the MeV, in particular in the ATU3 inserted between the H gene and the L gene of the MeV;
(iii) a third heterologous polynucleotide encoding at least one NEF antigen, or a fragment thereof, comprising an immunosuppressive domain (ISD), wherein the NEF antigen is mutated within its ISD domain, and is of a SIV or HIV, wherein the third heterologous polynucleotide is operatively cloned within the same or an additional transcription unit (ATU) as in (i) or (ii) inserted within the cDNA of the antigenomic (+) RNA, in particular an ATU located upstream the N gene of the MeV, in particular in the ATU1 inserted upstream the N gene of the MeV, or (iii bis) a third heterologous polynucleotide encoding at least one HBZ antigen, or a fragment thereof, or a mutated version thereof, of a HTLV, wherein the third heterologous polynucleotide is operatively cloned within the same or an additional transcription unit (ATU) as in (I or (ii) inserted within the cDNA of the antigenomic (+) RNA, in particular an ATU located upstream the N gene of the MeV, in particular in the ATU1 inserted upstream the N gene of the MeV,
wherein the mutation within the ISD domain of ENV reduces the immunosuppressive index of the ENV antigen; and wherein the mutation within the ISD domain of NEF reduces the immunosuppressive index of the antigen; and wherein the GAG, ENV, and NEF or GAG, ENV, and HBZ, or their respective immunogenic fragments or mutated versions thereof, all originate from the same virus type, in particular are from the same virus strain, more particularly from HIV-1, HIV-2 or from HTLV-1.

The nucleic acid construct may comprise a first heterologous polynucleotide inserted within a first ATU, a second heterologous polynucleotide sequence inserted into a second ATU at a distinct location from the first ATU, and a third heterologous polynucleotide inserted within a third ATU at a distinct location from the first and second ATUs. Alternatively, at least two heterologous polynucleotides may be inserted within the same ATU, or the three heterologous polynucleotides may be inserted within the same ATU.

In a third aspect, the invention concerns a combination of nucleic acids, wherein the combination comprises:
(a) a first nucleic acid construct which comprises a cDNA molecule encoding a full length antigenomic (+) RNA strand of a measles virus (MeV); and
(i) a first heterologous polynucleotide encoding at least one GAG antigen, a fragment thereof, or a mutated version thereof of a Simian Immunodeficiency Virus (SIV), a Human Immunodeficiency Virus (HIV), or a Human T-lymphotropic virus (HTLV), wherein the first heterologous antigen is operatively cloned within an additional transcription unit (ATU) inserted within the cDNA of the antigenomic (+) RNA, in particular an ATU located between the P gene and the M gene of the MeV, in particular in the ATU2 inserted between the P gene and the M gene of the MeV;
(ii) a second heterologous polynucleotide encoding at least one ENV antigen mutated within its immunosuppressive domain (ISD) of a Simian Immunodeficiency Virus (SIV), a Human Immunodeficiency Virus (HIV) or a Human T-lymphotropic virus (HTLV), wherein the second heterologous antigen is operatively cloned within the same or an additional transcription unit (ATU) as in (i) inserted within the cDNA of the antigenomic (+) RNA, in particular an ATU located between the H gene and the L gene of the MeV, in particular in the ATU3 inserted between the H gene and the L gene of the MeV; and
(b) a second nucleic acid construct comprising:
(i′) a second cDNA molecule encoding a full length antigenomic (+) RNA strand of a measles virus (MeV); and
(ii′) a third heterologous polynucleotide encoding at least one NEF antigen, or a fragment thereof, mutated within its ISD domain, of a SIV or HIV, wherein the third heterologous polynucleotide is operatively cloned within a transcription unit (ATU) inserted within the cDNA of the antigenomic (+) RNA, in particular an ATU located upstream the N gene of the MeV, in particular in the ATU1 inserted upstream the N gene of the MeV,
or (iii′ bis) a third heterologous polynucleotide encoding at least one HBZ antigen, or a fragment thereof, or a mutated version thereof, of a HTLV, wherein the third heterologous polynucleotide is operatively cloned within a transcription unit (ATU) inserted within the cDNA of the antigenomic (+) RNA, in particular an ATU located upstream the N gene of the MeV, in particular in the ATU1 inserted upstream the N gene of the MeV,
wherein the mutation within the ISD domain of ENV reduces the immunosuppressive index of the ENV antigen; wherein the mutation within the ISD domain of NEF reduces the immunosuppressive index of the NEF antigen; and wherein the GAG, ENV and NEF or HBZ antigens or their respective immunogenic fragments or mutated versions thereof, all originate from the same virus type, in particular are from the same virus strain, more particularly from HIV-1. HIV-2 or from HTLV-1.

By reducing the immunosuppressive index of the antigen(s), it should be understood that the mutated antigen elicits a higher immunogenicity as compared to its wild-type counterpart, but that the structure (i.e. the secondary and/or the tertiary structure; e.g. the folding) of the mutated antigen is kept or comparable with the structure of the wild-type antigen.

Particular nucleic acid constructs according to these embodiments are illustrated in FIG. 1 and FIG. 20 and in the examples of the invention. A sequence of nuclear acid construct corresponding to the cDNA molecule encoding a full length antigenomic (+) RNA strand of a measles virus (MeV) comprising a first heterologous polynucleotide encoding a SIV GAG protein and a second heterologous polynucleotide encoding a HIV ENV protein is illustrated on SEQ ID No: 32. A sequence of nuclear acid construct corresponding to the cDNA molecule encoding a full length antigenomic (+) RNA strand of a measles virus (MeV) comprising a first heterologous polynucleotide encoding a SIV NEF protein is illustrated on SEQ ID No: 33. A sequence of nuclear acid construct corresponding to the cDNA molecule encoding a full length antigenomic (+) RNA strand of a measles virus (MeV) comprising a first heterologous polynucleotide encoding a SIV GAG protein and a second heterologous polynucleotide encoding a mutated HIV ENV protein is illustrated on SEQ ID No: 40. A sequence of nuclear acid construct corresponding to the cDNA molecule encoding a full length antigenomic (+) RNA strand of a measles virus (MeV) comprising a first heterologous polynucleotide encoding a mutated SIV NEF protein is illustrated on SEQ ID No: 41. A sequence of nuclear acid construct corresponding to the cDNA molecule encoding a full length antigenomic (+) RNA strand of a measles virus (MeV) comprising a first heterologous polynucleotide encoding a wild type SIV NEF protein is illustrated on SEQ ID No: 42. A sequence of nuclear acid construct corresponding to the cDNA molecule encoding a full length antigenomic (+) RNA strand of a measles virus (MeV) comprising a first heterologous polynucleotide encoding a SIV GAG protein and a second heterologous polynucleotide encoding a wild type HIV ENV protein is illustrated on SEQ ID No: 43. A sequence of nuclear acid construct corresponding to the cDNA molecule encoding a full length antigenomic (+) RNA strand of a measles virus (MeV) comprising a first heterologous polynucleotide encoding a SIV GAG protein and a second heterologous polynucleotide encoding a mutated HIV ENV protein is illustrated on SEQ ID No: 44. A sequence of nuclear acid construct corresponding to the cDNA molecule encoding a full length antigenomic (+) RNA strand of a measles virus (MeV) comprising a first heterologous polynucleotide encoding a HTLV GAG protein and a second heterologous polynucleotide encoding a HTLV ENV protein is illustrated on SEQ ID No: 54.

The expression “encodes” in the above definition encompasses the ability of the nucleic acid construct, in particular the cDNA, to allow transcription of a full length antigenomic (+) RNA, said cDNA serving especially as a template for transcription and where appropriate translation for product expression into cells or cell lines. Hence, when the cDNA is a double stranded molecule, one of the strands has the same nucleotide sequence as the antigenomic (+) RNA of the measles virus with the first heterologous polynucleotide cloned within, except “U” nucleotides that are substituted by “T” nucleotides in the cDNA. The nucleic acid construct of the invention may comprise regulatory elements controlling the transcription of the coding sequences, in particular promoters and termination sequences for the transcription, and possibly enhancer and other cis-acting elements. These regulatory elements may be heterologous with respect to the heterologous polynucleotide issued or derived from IV gene(s) or HTLV gene(s), in particular may be the regulatory elements of the measles virus strain.

The expression “operatively cloned”, which can be substituted by the expression “operatively linked”, refers to the functional cloning, or insertion, of a heterologous polynucleotide within the nucleic acid construct of the invention such that said polynucleotide and nucleic acid construct are effectively, or efficiently, transcribed and if appropriate translated, in particular in cells, cell line, host cell used as a part of a rescue system for the production of recombinant infectious MeV particles or MeV expressing at least one antigen, or at least one protein, or at least one polypeptide, or at least an antigenic fragment thereof, of IV or HTLV. In other words, the nucleic acid construct of the invention allows the production, when placed in appropriate conditions, of an infectious antigenomic (+) RNA capable of producing at least one antigen, or at least one protein, or at least one polypeptide, or at least an antigenic fragment thereof, of HIV or HTLV.

In a particular embodiment of the invention, the nucleic acid construct comprising the cDNA encoding the nucleotide sequence of the full-length infectious antigenomic (+) RNA strand of MeV but without the operatively cloned heterologous antigen complies with the rule of six (6) of the measles virus genome. In other words, the cDNA encoding the nucleotide sequence of the full-length, infectious antigenomic (+) RNA strand of MeV is a polyhexameric cDNA.

The organization of the genome of measles viruses and its replication and transcription process have been fully identified in the prior art and are especially disclosed in Horikami S. M. and Moyer S. A. (20) or in Combredet C. et al (21) for the Schwarz vaccination strain of the virus or for broadly considered negative-sense RNA viruses, in Neumann G. et al (22).

The “rule of six” is expressed in the fact that the total number of nucleotides present in a nucleic acid representing the MeV (+) strand RNA genome or in the nucleic acid constructs comprising the same is a multiple of six. The “rule of six” has been acknowledged in the state of the art as a requirement regarding the total number of nucleotides in the genome of the measles virus, which enables efficient or optimized replication of the MeV genomic RNA. In the embodiments of the present invention defining a nucleic acid construct that meets the rule of six, said rule applies to the nucleic acid construct specifying the cDNA encoding the full-length MV (+) strand RNA genome. In this regard the rule of six applies individually to the cDNA encoding the nucleotide sequence of the full-length infectious antigenomic (+) RNA strand of the measles virus possibly but not necessarily to the polynucleotide cloned into said cDNA and encoding at least one polypeptide of the IV or HTLV.

The nucleic acid constructs of the invention are in particular purified DNA molecules, obtained or obtainable by recombination of at least one polynucleotide of MeV and at least one, or several, antigen(s) of the IV or HTLV, operably cloned or linked together.

According to the invention, the nucleic acid constructs are prepared by cloning an antigen, a polynucleotide, or several antigens or polynucleotides, encoding at least one antigen, polypeptide, a protein, an antigenic fragment thereof, or a mutated version thereof, wherein the antigen, or polypeptide, protein, fragment and mutated version thereof, is selected from the group consisting of the GAG, ENV and NEF, issued or derived from HIV and SIV or GAG, ENV and HBZ issued or derived from HTLV-1, in the cDNA encoding the full-length antigenomic (+) RNA of the measles virus. Constructs according to the invention are illustrated on FIG. 1 and FIG. 20. Alternatively, a nucleic acid construct of the invention may be prepared using steps of synthesis of nucleic acid fragments or polymerization from a template, including by PCR. The polynucleotide(s) and nucleic acid construct of the invention may rather be prepared in accordance with any known method in the art and in particular may be cloned and produced in a producing cell, obtained by polymerization or may be synthesized. Alternatively, any one of these antigens or polynucleotides may be a cDNA issued from the genomic RNA of the IV or HLTV, after retrotranscription, said cDNA being either the full genomic cDNA or a fragment thereof, and encoding a polypeptide of the IV or HTLV.

Additional Transcription Units (ATUs)

The heterologous polynucleotide(s), in particular IV gag, env and/or nef gene(s), in particular HTLV-1 gag, env and/or hbz gene(s), is/are inserted, especially cloned, within an additional transcription unit (ATU) inserted in the cDNA of the MeV. ATU sequences are known from the skilled person and comprise, for use in steps of cloning into cDNA of MeV, cis-acting sequences necessary for MeV-dependent expression of a transgene, such as a promoter of the gene preceding, in MeV cDNA, the insert represented by the polynucleotide encoding the IV or HTLV polypeptides inserted into a multiple cloning sites cassette of said ATU. The ATU may be further defined as disclosed by Billeter et al. in WO 97/06270. Three ATUs are represented on FIGS. 1A and 1B. An ATU may also be defined as multiple cloning cassette inserted within the cDNA of the MeV, in particular between the P-M intergenic region of the MeV genome, and/or between the intergenic H-L region of the MeV genome. An ATU may contain cis-acting sequences necessary for the transcription of the P gene of MeV, in particular cis-acting sequence issued or originates from a MeV. The different ATUs in particular ATU1 and ATU2 may be identical regarding their nucleic acid sequence. ATUs are generally located between two CTT codons corresponding respectively to the start and stop codons of the polymerase. ATUs may further comprise a ATG and a TAG codons corresponding respectively to the start and stop codons for translation of the heterologous polynucleotide cloned within the ATU. Alternatively, ATUs are located between a ATG and a TAG codons corresponding respectively to the start and stop codons for translation of the heterologous polynucleotide cloned within the ATU. In a preferred embodiment of the invention, an ATU is a polynucleotide comprising or consisting of SEQ ID No: 24, wherein a polynucleotide sequence encoding an antigen as defined herein is inserted.

SEQ ID No: 24

SEQ ID No: 24 is an ATU sequence located within the cDNA molecule encoding a full-length antigenomic (+) RNA strand of a measles virus. CTT codons corresponding respectively to the start and stop codons of the polymerase are in bold. ATG and TAG codons corresponding to the start and stop codons for translation of the heterologous polynucleotide cloned within the ATU are underlined.

CTTAGGAACCAGGTCCACACAGCCGCCAGCCCATCAacgcgtacgATG*TAGg cgcgcagcgcttagacgtctcgcgaTC_GATACTAGTACAACCTAAATCCATTATAAAAA ACTT wherein the * corresponds to the location of the heterologous, optionally codon-optimized, sequence polynucleotide encoding at least one HTLV polypeptide to be inserted.

An ATU comprising a heterologous polynucleotide encoding the SIV GAG polypeptide is for example located between positions 3539 and 5074 on SEQ ID No: 32. An ATU comprising a heterologous polynucleotide encoding the HIV ENV polypeptide is for example located between positions 10991 and 13335 on SEQ ID No: 32. An ATU comprising a heterologous polynucleotide encoding the SIV NEF polypeptide is for example located between positions 272 and 1126 on SEQ ID No: 33.

An ATU comprising a heterologous polynucleotide encoding the HTLV GAG polypeptide is for example located between positions 3541 and 4830 on SEQ ID No: 54. An ATU comprising a heterologous polynucleotide encoding the HTLV ENV polypeptide is for example located between positions 10750 and 12216 on SEQ ID No: 54.

Such examples of localization of the heterologous polynucleotides within the cDNA of a measles virus are illustrated on FIG. 16 and FIG. 21.

An ATU (known under reference ATU2) is located between the P and M genes of the MeV. Another ATU (known under reference ATU1) is located upstream the gene N of the MeV. Another ATU (known under reference ATU3) is located between the genes H and L of MeV. It has been observed that the transcription of the viral RNA of MeV follows a gradient from the 5′ to the 3′ end. This explains that, depending on where the heterologous polynucleotide is inserted, its level of expression will vary and be more or less efficient if inserted within ATU1, ATU2 or ATU3.

According to an aspect of the invention, the nucleic acid construct comprises a first and a second heterologous polynucleotides encoding GAG and ENV operatively cloned within one ATU or different ATUs (i.e. the second polynucleotide encoding the ENV antigen is at a location distinct from the location of the first cloned heterologous polynucleotide, said another ATU being in particular the ATU3). In other words, the polynucleotides inserted within the full length antigenomic (+) RNA strand of a measles virus (MeV) may be located within the same ATU or different ATU. In a more particular embodiment, the first and second polynucleotides are inserted within different ATUs, more particularly within ATU2 and ATU3. In a preferred embodiment, the polynucleotide encoding GAG is inserted within ATU2, and the polynucleotide encoding ENV is inserted within ATU3. In particular, the heterologous polynucleotide encodes ENV and GAG of HIV, especially HIV-1.

Antigens Encoded by the Nucleic Acid Construct(s) of the Invention

The term “antigen” is used interchangeably with the terms “polypeptide” or “protein” or “antigenic fragment”, which also refers to mutated version thereof and/or truncated version thereof, and defines a molecule resulting from a concatenation of amino acid residues. In particular, the polypeptides disclosed in the application originate from the expression of gag, env and nef genes of either HIV and SIV; or from the expression of gag, env and/or hbz genes of HTLV-1, and are antigens, proteins, structural proteins, or antigenic fragments thereof, that may be identical to native proteins (i.e. wild-type proteins) or alternatively that may be derived thereof by mutation, including by substitution (in particular by conservative amino acid residues) or by addition of amino acid residues or by secondary modification after translation or by deletion of portions of the native proteins(s) resulting in fragments having a shortened size with respect to the native protein of reference. Fragments are encompassed within the present invention to the extent that they bear epitopes of the native protein suitable for the elicitation of an immune response in a host in particular in a human host, including a child host, preferably a response that enables the protection against IV or HTLV, in particular HIV infection, or against IV or HTLV, in particular HIV, associated disease. Epitopes are in particular of the type of T epitopes involved in elicitation of Cell Mediated Immune response (CMI response). T epitopes are involved in the stimulation of T cells through presentation of the T-cell epitope which can bind on MHC class I and II molecules, leading to the activation of T cells. Epitopes may alternatively be of type B, involved in the activation of the production of antibodies in a host to whom the protein has been administered or in whom it is expressed following administration of the infectious replicative particles of the invention. Fragments may have a size representing more than 50% of the amino-acid sequence size of the native protein of IV or HTLV, in particular HIV, SIV, or HTLV-1, preferably at least 90% or 95%. Polypeptide may have at least 50% identity with the native protein of HIV, in particular HIV-1, preferably at least 60%, preferably at least 70%, preferably at least 85% or 95%. A fragment may also correspond to the above definition, but further comprising a mutated immunosuppressive domain when applicable (e.g. when the antigen is either ENV or NEF). Native IV proteins may correspond to the wild type proteins corresponding to SEQ ID No: 1 for SIV GAG; SEQ ID No: 2 for HIV-1 GAG; SEQ ID No: 3 for HIV-2 GAG; SEQ ID No: 4 for SIV GAG-pro; SEQ ID No: 5 for HIV-1 GAG-pro; SEQ ID No: 6 for HIV-2 GAG-pro. Mutated version of the antigens may correspond to a polypeptide having at least 70%, more preferably at least 80%, and most preferably at least 90% or at least 95% identify with the wild type proteins corresponding to SEQ ID No: 1 for SIV GAG; SEQ ID No: 2 for HIV-1 GAG; SEQ ID No: 3 for HIV-2 GAG; SEQ ID No: 4 for SIV GAG-pro; SEQ ID No: 5 for HIV-1 GAG-pro; SEQ ID No: 6 for HIV-2 GAG-pro. Native HTLV proteins may correspond to the wild type proteins corresponding to SEQ ID No: 45 for HTLV-1 GAG; SEQ ID No: 47 for HTLV-1 ENV; SEQ ID No: 52 for HTLV-2 ENV, SEQ ID No: 46 for HTLV-1 GAG-pro, SEQ ID No: 55 for HTLV-1 HBZ, SEQ ID No: 50 for TAX. Mutated version of the antigens may correspond to a polypeptide having at least 70%, more preferably at least 80%, and most preferably at least 90% or at least 95% identify with the wild type proteins corresponding to SEQ ID No: 45 for HTLV-1 GAG; SEQ ID No: 46 for HTLV-1 GAG-pro; SEQ ID No: 47 for HTLV-1 ENV; SEQ ID No: 55 for HTLV-1 HBZ, and SEQ ID No: 50 for TAX.

In a particular embodiment of the invention, each polynucleotide operatively cloned within the cDNA of the antigenomic (+) RNA encodes polypeptides comprising epitopes located within one of the IV or HTLV polypeptide(s). According to this embodiment, the epitope sequence(s) share(s) 100% identity with the epitope sequence(s) of the native HIV or SIV, or HTLV-1, selected proteins. Such epitopes are listed in the Immune Epitope database and analysis resource (www.iedb.orq). Within the polypeptide(s) of the HIV, FIV, SIV or HTLV-1 encoded by the polynucleotide and having an epitope sequence(s) as defined herein, amino acid residue that does not belong to any epitope may be different from its counterpart in the sequence of the native (i.e. wild-type) HIV, SIV or HTLV-1 protein(s).

By “polypeptide of HIV, SIV or FIV” or “polypeptide of HTLV” is meant an “antigen” or a “polypeptide” as defined herein (either a polypeptide, an antigen, a protein, an antigenic fragment thereof, or a mutated version thereof as compared to a wild-type version of the polypeptide), the amino acid sequence of which is identical to or derived from a counterpart in a strain of HIV, SIV, or HTLV, especially HIV-1, or from a consensus sequence of HIV or HTLV, especially HIV-1, including a polypeptide which is a native mature or precursor of protein of HIV, SIV or HTLV, or is an antigenic fragment thereof or a mutant thereof as defined herein in particular an antigenic fragment or a mutant having at least 50%, at least 80%, in particular advantageously at least 90% or preferably at least 95% amino acid sequence identity to a naturally occurring HIV, or SIV proteins GAG, ENV and NEF, or to a naturally occurring HTLV proteins GAG, ENV and HBZ.

HIV amino acid sequence identity can be determined by alignment by one of skill in the art using manual alignments or using the numerous alignment programs available (for example, BLASTP—http://blast.ncbi.nlm.nih.gov/). Fragments or mutants of GAG, ENV and NEF polypeptides of the invention may be defined with respect to the particular amino acid sequences illustrated herein, especially the amino acid sequences from the group consisting of SEQ ID No: 1, SEQ ID No: 2, SEQ ID No: 3 for GAG, SEQ ID No: 4, SEQ ID No: 5, SEQ ID No: 6 for GAG-pro, SEQ ID No: 7, SEQ ID No: 8, SEQ ID No: 9, SEQ ID No: 10, SEQ ID No: 11, SEQ ID No: 12, SEQ ID No: 13, for ENV and SEQ ID No: 14, SEQ ID No: 15, SEQ ID No: 16, SEQ ID No: 17, SEQ ID No: 18 and SEQ ID No: 19 for NEF. In a particular embodiment of the invention, the polypeptides share at least 50%, at least 80%, in particular advantageously at least 90% or preferably at least 95% amino acid sequence identity with their native proteins of HIV or SIV, or with the polypeptides of SEQ ID No: 1, SEQ ID No: 2, SEQ ID No: 3 for GAG, SEQ ID No: 4, SEQ ID No: 5, SEQ ID No: 6 for GAG-pro, SEQ ID No: 7, SEQ ID No: 9, SEQ ID No: 12 and SEQ ID No: 16 for ENV and SEQ ID No: 14, SEQ ID No: 16 and SEQ ID No: 18 for NEF. Alternatively, the native proteins GAG, GAG-pro, ENV and NEF of HIV or SIV may be found in databases, such as but not limited to the HIV Sequence Database in Los Alamos, which collects all sequences and focuses on annotation and data analysis, and the HIV RT/Protease Sequence Database in Stanford.

HTLV amino acid sequence identity can be determined by alignment by one of skill in the art using manual alignments or using the numerous alignment programs available (for example, BLASTP—http://blast.ncbi.nlm.nih.gov/). Fragments or mutants of GAG and ENV HTLV polypeptides of the invention may be defined with respect to the particular amino acid sequences illustrated herein, especially the amino acid sequences from the group consisting of SEQ ID No: 45 for GAG, SEQ ID No: 46 for GAG-pro, SEQ ID No: 47 for wild-type HTLV-1-ENV; SEQ ID No: 52 for HTLV-2 ENV; SEQ ID No: 48 for HTLV-1 ENV mutated within its IS domain, SEQ ID No: 53 for HTLV-2 mutated within its IS domain; SEQ ID No: 55 for wild type HBZ, SEQ ID No: 49 for mutated HBZ, and SEQ ID No: 50 for TAX. In a particular embodiment of the invention, the polypeptides share at least 50%, at least 80%, in particular advantageously at least 90% or preferably at least 95% amino acid sequence identity with their native proteins of HTLV, in particular of HTLV-1 or HTLV-2, or with the polypeptides SEQ ID No: 45 for GAG, SEQ ID No: 46 for GAG-pro, SEQ ID No: 47 for ENV, and SEQ ID No: 55 for HBZ, and SEQ ID No: 50 for TAX. Alternatively, the native proteins GAG, GAGpro, ENV, HBZ and TAX of HTLV may be found in databases which collect sequences.

GAG Antigens

The first polynucleotide encodes a GAG antigen, an immunogenic fragment thereof, or a mutated version thereof. The antigen issued or derived from GAG corresponds to the definition of the “antigen” as described therein. The polynucleotide encoding GAG may be issued from a SIV strain, a HIV strain or a HTLV strain. The GAG antigen may correspond to the GAG protein or to the GAG-pro protein, which correspond to the pre-pro-protein of GAG. In a particular embodiment, the polynucleotide encoding GAG is issued from HTLV-1, HTLV-2 HTLV-3, HIV-1 or HIV-2, more particularly from HIV-1. The polynucleotide may comprise or consist of a nucleotide sequence encoding an antigen with a sequence selected from the group consisting of SEQ ID No: 1 (GAG-SIV), SEQ ID No: 4 (GAG-pro-SIV), SEQ ID No: 2 (GAG-HIV-1), SEQ ID No: 3 (GAG-HIV-2), SEQ ID No: 5 (GAGpro-HIV-1), SEQ ID No: 6 (GAGpro-HIV-2). The encoded antigen may consist in an amino acid sequence selected from the group consisting of SEQ ID No: 1 (GAG-SIV), SEQ ID No: 4 (GAG-pro-SIV), SEQ ID No: 2 (GAG-HIV-1), SEQ ID No: 3 (GAG-HIV-2), SEQ ID No: 5 (GAGpro-HIV-1), SEQ ID No: 6 (GAGpro-HIV-2). The first polynucleotide may be inserted within the cDNA of the antigenomic (+) RNA strand of the MeV within any ATU, in particular within ATU 1, ATU 2, or ATU 3. In a particular embodiment, the first polynucleotide is inserted within ATU 2. The polynucleotide encoding GAG may be issued from any HTLV strain. The GAG antigen may correspond to the GAG protein or to the GAG-pro protein, which correspond to the pre-pro-protein of GAG. In a particular embodiment, the polynucleotide encoding GAG is issued from HTLV-1, HTLV-2 or HTLV-3, more particularly from HTLV-1. The polynucleotide may comprise or consist of a nucleotide sequence encoding an antigen with a sequence selected from the group consisting of SEQ ID No: 45 (GAG-HTLV) or SEQ ID No: 46 (GAGpro-HTLV). The encoded antigen may consist in an amino acid sequence selected from the group consisting of SEQ ID No: 45 (GAG-HTLV) or SEQ ID No: 46 (GAGpro-HTLV). The first polynucleotide may be inserted within the cDNA of the antigenomic (+) RNA strand of the MeV within any ATU, in particular within ATU 1, ATU 2, or ATU 3. In a particular embodiment, the first polynucleotide is inserted within ATU 2.

Mutated and Wild Type NEF Antigens (from HIV) and Mutated and Wild Type ENV Antigens (from HIV and HTLV)

In the present description, the expression “mutated NEF” or “mutated ENV” corresponds to a NEF antigen or a ENV antigen with reduced immunosuppressive index as compared to a wild type NEF antigen or wild type ENV antigen. Wild type NEF antigen and wild type ENV antigen may partially, or fully, correspond to the amino acid sequence set forth in SEQ ID No: 7 (ENV SIV), SEQ ID No: 9 (ENV-HIV-1), SEQ ID No: 12 (ENV-HIV-2), SEQ ID No: 14 (NEF-SIV), SEQ ID No: 16 (NEF-HIV-1) or SEQ ID No: 18 (NEF-HIV-2). A mutated ENV antigen or NEF antigen may correspond to an antigen having at least 70%, preferably at least 80%, more preferably at least 90% of identity with the wild type amino acid sequences recited herein, and harboring reduced or no immunosuppressive activity.

The second polynucleotide encodes a ENV antigen, or an immunogenic fragment thereof, mutated within its immunosuppressive domain. An immunosuppressive domain (ISD) is a conserved region in envelope genes (env) of HIV, SIV and HTLV. In the invention, the IS domain refers to a specific domain in which a mutation can affect the immunosuppressive property of the ENV protein. The localization of an IS domain can be determined in all ENV proteins of viruses as described in Benit et al. (23). In a broad meaning, the IS domain is defined by its structure and its localization, irrespective of the fact that it possesses or not an immunosuppressive activity. The immunosuppressive properties of the mutated ENV proteins according to the invention may be measured according to an in vivo procedure to assay the immunosuppressive activity of a ENV protein disclosed previously (15, 18).

A mutation within the ISD may correspond to the substitution or deletion of at least one amino acid residue located within the ISD of ENV. Mutated HIV ENV are disclosed in International patent publication No. WO2013083799, wherein several mutations within the ISD of HIV ENV are disclosed, illustrating which mutations allow the expression of more immunogenic viruses expressing a mutated ENV. A mutation within the ISD may correspond to the substitution or deletion of at least one amino acid residue located within the ISD of ENV. Mutated HTLV ENV are disclosed in International patent publication No. WO2005095442, wherein several mutations within the ISD of HTLV ENV are disclosed, illustrating which mutations allow the expression of more immunogenic viruses expressing a mutated ENV.

Any mutated HIV ENV disclosed therein may be contemplated for being encoded by the heterologous polynucleotide inserted within a nucleic acid construct of the invention. More particularly, mutation within the immunosuppressive domain of ENV may consist in the substitution of amino acid residue(s) located within the IS domain of ENV. As an example, the amino acid residue Y located on position 589 of SEQ ID No: 9 may be substituted by amino acid residue R. Such a mutation corresponds to the protein ENV of SEQ ID No: 10. Several substitutions may also be performed within the IS domain of ENV. As an example, the amino acid residue Y located on position 589 of SEQ ID No: 9 may be substituted by amino acid residue R and the amino acid residue K located on position 620 of SEQ ID No: 9 may be substituted by amino acid residue A, G or S. A HIV-1 ENV protein comprising both substitutions (Y589R and K620A) may correspond to the amino acid sequence of SEQ ID No: 11. Other mutations may be contemplated; as examples, amino acid residue Y on position 589 of SEQ ID No: 9 may be substituted by amino acid residues G, L, A or F; amino acid residue L on position 590 of SEQ ID No: 9 may be substituted by amino acid residue R.

When the ENV antigen is issued from HIV-2, the mutation of the IS domain may correspond to the substitution of amino acid residue L located on position 582 of SEQ ID No: 12 by amino acid residue R. such a mutated HIV-2 ENV antigen may correspond to the amino acid sequence illustrated on SEQ ID No: 13. When the ENV antigen is issued from SIV, the mutation of the IS domain may correspond to the substitution of amino acid residue L located on position 600 of SEQ ID No: 7 by amino acid residue R. such a mutated SIV ENV antigen may correspond to the amino acid sequence illustrated on SEQ ID No: 8. An ENV antigen with mutated ISD reduces the immunosuppression induced by wild-type ENV. A ENV antigen with a mutated ISD useful in the present invention corresponds to a mutated ENV which presents a reduced or lowered immunosuppressive index as compared to its wild-type counterpart(s).

The immunosuppressive index may be measured according to the method illustrated on the examples of the invention, in particular by the tumor rejection assays illustrated in the working examples of the present description. Briefly, a wild-type (wild type ENV protein) or modified nucleic acid expressing the protein to be tested (mutated ENV protein) is transduced in tumor cell lines such as MCA205 and CL8.1, in particular MC1205, cell lines by known methods. The tumor cells expressing the protein to be tested are then injected especially subcutaneous (s.c.) injection to a host, generally mice. Following said injection, the establishment of tumor or, to the contrary, its rejection, is determined and the tumor area is measured. Tumor establishment was determined by palpation and tumor area (mm<2>) was determined by measuring perpendicular tumor diameters. Immunosuppression index is defined as i=(Senv-Snone)/Snone, wherein Senv is the maximum area reached by a tumor expressing an envelope protein and Snone is the maximum area reached by a tumor not expressing ENV protein (negative control). The above-defined ratio relative to the immunosuppressive index can be less than 0.2, and can even have a negative value. In the invention, an antigen with reduced or no immunosuppressive properties may mean that the mutated antigen according to the invention has an immunosuppressive index less than about 0.2. The mutation(s) within the immunosuppressive domain of the ENV proteins is (are) sufficient to decrease the immunosuppressive activity of the mutated ENV protein with respect to the corresponding wild type ENV. However, it might be advantageous that another amino acid be also mutated because it ensures that the structure of the mutated ENV protein is essentially conserved with respect to the corresponding wild type ENV protein. Therefore, a mutated ENV antigen may have substantially the same structure as its wild type counterpart (i.e. non-mutated antigen).

The antigen issued or derived from ENV corresponds to the definition of the “antigen” as described therein. The polynucleotide encoding ENV may be issued from a SIV strain, a HIV strain or a HTLV strain.

In a particular embodiment, the ENV polynucleotide is issued from HTLV-1 or HIV, more particularly from HIV-1. The encoded antigen may correspond to an amino acid sequence selected from the group consisting of SEQ ID No: 8 (mutated ENV-SIV), SEQ ID No: 10 (single mutated ENV-HIV-1), SEQ ID No: 11 (double mutated ENV-HIV-1), SEQ ID No: 13 (mutated ENV-HIV-2). In particular embodiments, when the nucleic acid construct is for example used in a prime-boost regimen, the nucleic acid construct used during the boost may encode for a ENV of SEQ ID No: 21 (ENV-HIV cons B) or SEQ ID No: 20 (ENV SF162-HIV). The second polynucleotide may be inserted within the cDNA of the antigenomic (+) RNA strand of the MeV within any ATU, in particular within ATU 1, ATU 2, or ATU 3. In a particular embodiment, the first polynucleotide is inserted within ATU 3.

Mutated HIV-1 ENV protein may have the amino acid residue Y located on position 589 of SEQ ID No: 9 substituted by amino acid residue R. Such a mutation corresponds to the protein ENV of SEQ ID No: 10. Several substitutions may also be performed within the IS domain of ENV. As an example, the amino acid residue Y located on position 589 of SEQ ID No: 9 may be substituted by amino acid residue R and the amino acid residue K located on position 620 of SEQ ID No: 9 may be substituted by amino acid residue A, G or S. A HIV-1 ENV protein comprising both substitutions (Y589R and K620A) may consist in the amino acid sequence of SEQ ID No: 11. Other mutations may be contemplated; as examples, amino acid residue Y on position 589 of SEQ ID No: 9 may be substituted by amino acid residues G, L, A or F; amino acid residue L on position 590 of SEQ ID No: 9 may be substituted by amino acid residue R. When the ENV antigen is issued from HIV-2, the mutation of the IS domain may correspond to the substitution of amino acid residue L located on position 582 of SEQ ID No: 12 by amino acid residue R. Such a mutated HIV-2 ENV antigen may correspond to the amino acid sequence illustrated on SEQ ID No: 13. When the ENV antigen is issued from SIV, the mutation of the IS domain may correspond to the substitution of amino acid residue L located on position 600 of SEQ ID No: 7 by amino acid residue R. such a mutated SIV ENV antigen may correspond to the amino acid sequence illustrated on SEQ ID No: 8.

In a particular embodiment, the fragment of the ENV antigen comprises or consists of the envelope subunit gp41, which corresponds to the extracellular domain of the HIV envelope subunit, deleted of the immune-dominant region (cluster I), but comprising the mutated immunosuppressive domain, the so-called 3S motif and the MPER (membrane-proximal external region). Such an ENV antigen are for example encoded by the nucleic acid construct of the invention of SEQ ID No: 43 and SEQ ID No: 44.

The inventors have shown that the MeV comprising the polynucleotides for GAG and mutated ENV-GP41 as above results in the production of VLP associated antigens and a strong cellular immune response in mice (see for example the results illustrated on FIG. 17 and FIG. 18).

The polynucleotide encoding ENV may be issued from any HTLV strain. In a particular embodiment, the polynucleotide is issued from HTLV-1. The encoded antigen may correspond to an amino acid sequence selected from the group consisting of SEQ ID No: 48 (mutated ENV-HTLV-1) or SEQ ID No: 53 (Mutated ENV-HTLV-2). The second polynucleotide may be inserted within the cDNA of the antigenomic (+) RNA strand of the MeV within any ATU, in particular within ATU 1, ATU 2, or ATU 3. In a particular embodiment, the first polynucleotide is inserted within ATU 3.

In a particular embodiment, the fragment of the ENV antigen comprises or consists of the envelope subunit gp21, which corresponds to the extracellular domain of the HTLV envelope transmembrane (TM) subunit, comprising the mutated immunosuppressive domain.

MeV comprising the polynucleotides for GAG and mutated ENV-GP21 as above results in the production of VLP associated antigens that should induce strong cellular immune responses (see for example the results illustrated on FIGS. 21 and 22).

Mutated HTLV-1 ENV protein may have the amino acid residue Q on position 389 of SEQ ID No: 47 substituted by amino acid residue R. An additional substitution may be performed within the IS domain of ENV, the amino acid residue A located on position 395 of SEQ ID No: 47 may be substituted by amino acid residue F. A HTLV-1 ENV protein comprising both substitutions (Q389R and A395F) is the amino acid sequence of SEQ ID No: 48.

Mutated HTLV-2 ENV protein may have the amino acid residue Q on position 385 of SEQ ID No: 52 substituted by amino acid residue R. Another or an additional substitution may be performed within the IS domain of ENV of HTLV-2, the amino acid residue A located on position 391 of SEQ ID No: 52 may be substituted by amino acid residue F. A HTLV-2 ENV protein comprising both substitutions (Q385R and A391F) is the amino acid sequence of SEQ ID No:53.

Any mutated NEF antigen disclosed in the present application may be encoded within a nucleic acid construct according to the invention. The polynucleotide encoding NEF may be issued from a SIV strain or a HIV strain. In a particular embodiment, the polynucleotide is issued from HIV-1. The encoded antigen may correspond to an amino acid sequence selected from the group consisting of SEQ ID No: 15 (mutated NEF-SIV), SEQ ID No: 17 (mutated NEF-HIV-1) or SEQ ID No: 19 (mutated NEF-HIV-2). The third polynucleotide may be inserted within the cDNA of the antigenomic (+) RNA strand of the MeV within any ATU, in particular within ATU 1, ATU 2, or ATU 3. In a particular embodiment, the first polynucleotide is inserted within ATU 1.

More particularly, mutation within the immunosuppressive domain of NEF may consist in the substitution of amino acid residue(s) located within the IS domain of NEF. The immunosuppressive index may be measured according to the method illustrated on the examples of the invention, in particular the tumor rejection assays as recalled above for the ENV antigen. In a particular embodiment, the mutation consists of at least one amino acid substitution in the immunosuppressive domain of a NEF protein, which modulates the immunosuppressive property of said protein.

As an example, mutation within the immunosuppressive domain of NEF may consist in the substitution of amino acid residue(s) located within the IS domain of NEF. A mutation within the ISD of NEF may correspond to the substitution or deletion of at least one amino acid residue located within the ISD of NEF. A NEF antigen with a mutated immunosuppressive domain has reduced or no immunosuppressive properties, as described here above, but kept other functional properties as compared to a wild type NEF antigen.

As examples, when the NEF antigen is issued from SIV, the amino acid residue E located on position 125 of SEQ ID No: 14 may be substituted by amino acid residue R; such a mutated NEF antigen may correspond to a protein of amino acid sequence SEQ ID No: 15; when the NEF antigen is issued from HIV-1, the amino acid residue E located on position 93 of SEQ ID No: 16 may be substituted by amino acid residue R; such a mutated NEF antigen may correspond to a protein of amino acid sequence SEQ ID No: 17; when the NEF antigen is issued from HIV-2, the amino acid residue E located on position 125 of SEQ ID No: 18 may be substituted by amino acid residue R; such a mutated NEF antigen may correspond to a protein of amino acid sequence SEQ ID No: 19. Any polynucleotide encoding a NEF antigen may also comprise a peptide signal allowing the cellular exportation (i.e. secretion) of the NEF antigen. The peptide signal may be any peptide signal known for allowing the exportation of a protein. The NEF antigen of SEQ ID No: 22 corresponds to a wild-type HIV-1-NEF (i.e. not mutated within its IS domain) with a peptide signal, while the NEF antigen of SEQ ID No: 23 corresponds to a HIV-1-NEF antigen with a mutated ISD (substitution of the amino acid residue E located on position 93 of SEQ ID No: 17 by amino acid residue R) and a peptide signal. The NEF antigen may also be mutated for avoiding myristoylation.

Within the nucleic acid construct, the polynucleotides encoding GAG and ENV are issued or derived from genes issued from a same virus species, i.e. both are issued or derived from a HIV, a SIV or a HTLV, in particular from HIV-1 or HIV-2 or from HLTV-1, and more particularly from HIV-1. In a particular embodiment, the GAG and ENV antigens (or the polynucleotides encoding the GAG and ENV antigens) are both issued or derived from a HIV, in particular from HIV-1 or HIV-2.

Within the nucleic acid construct, the polynucleotides encoding GAG, ENV and NEF are issued or derived from genes issued from a same virus species, i.e. both are issued or derived from a HIV or a SIV, in particular from HIV-1 or HIV-2, and more particularly from HIV-1. In a particular embodiment, the GAG, ENV and NEF antigens (or the polynucleotides encoding the GAG and ENV antigens) are all issued or derived from a HIV, in particular from HIV-1 or HIV-2.

Mutated and Wild Type HBZ Antigens (from HTLV)

In an embodiment of the invention, the third heterologous polynucleotide encodes at least a fragment of a HBZ antigen. HBZ refers to HTLV bZIP factor. The wild type version of HBZ may correspond to the protein listed as UniProt reference P0C746, or of SEQ ID No: 55. In the present description, the HBZ antigen may correspond to at least a fragment of the mutated HBZ of SEQ ID No: 49, or a fragment or a further mutated version thereof. Alternatively, a fragment or a mutated version of wild type HBZ of SEQ ID No: 55 may be encoded by the heterologous polynucleotide inserted within the nucleic acid construct of the invention. The mutated HBZ may correspond to a wild type HBZ, in particular of SEQ ID No: 49, wherein 2 leucine amino-acid residues localized at the N-terminal part are substituted by alanine residues (L27A/L28A) to prevent the activation of the transforming growth factor beta/Smad pathway, therefore reducing the oncogenic properties of wild type HBZ of SEQ ID No: 55.

The third heterologous polynucleotide may encode at least a fragment of a HBZ antigen of HTLV, in particular comprising or consisting of the amino acid sequence of SEQ ID No: 49, associated with at least a fragment of a TAX antigen. In particular, the third heterologous polynucleotide encodes a HBZ antigen associated with a TAX antigen of the amino acid residue of SEQ ID No: 49 and SEQ ID No: 50 respectively; or of SEQ ID No: 51 wherein the HBZ and TAX antigen are associated to a GPI anchor.

A mutated HBZ antigen may correspond to an antigen having at least 70%, preferably at least 80%, more preferably at least 90% of identity with the wild type amino acid sequences recited herein (SEQ ID No: 49), and harboring reduced or no immunosuppressive activity. The HBZ antigen may also be associated with another antigen issued from HTLV, and in particular TAX, more particularly TAX of the amino acid residues sequence of SEQ ID No: 50. Such a fusion protein comprising the antigens of HBZ and TAX is for example illustrated in the amino acid residues sequence set forth in SEQ ID No: 51. A mutated TAX antigen may be encoded in the nucleic acid construct of the invention. A mutated TAX may correspond to a TAX antigen having at least 70%, preferably at least 80%, more preferably at least 90% of identity with the wild type amino acid sequences recited herein (SEQ ID No: 50). In a particular embodiment, the HBZ antigen is associated with a fragment of the TAX antigen comprising at least two epitopes recognized by T cells, in particular by human T cells.

Within the nucleic acid construct, the polynucleotides encoding GAG, ENV and HBZ (and TAX when applicable) are issued or derived from genes issued from a same virus species, i.e. both are issued or derived from the same HTLV, in particular from HTLV-1 or HTLV-2 or HTLV-3, and more particularly from HTLV-1. In a particular embodiment, the GAG, ENV and HBZ (and TAX when applicable) antigens (or the polynucleotides encoding the GAG, ENV, HBZ (and TAX when applicable) antigens) are all issued or derived from the same HTLV, in particular from HTLV-1 or HTLV-2 or HTLV-3.

According to a particular embodiment of the invention, the NEF or HBZ antigen, the ENV antigen and the GAG antigen (including the GAGpro antigen) correspond to full length proteins, possibly mutated within their IS domain when applicable.

According to one aspect of the invention, a polynucleotide encoding at least one antigen of HIV, SIV or HTLV, in particular HIV-1, is issued or derived from the genome of isolated and purified wild strain(s) of HIV, SIV or HTLV, in particular HIV-1, or are derived from a consensus sequence, such as a consensus HIV-1 genome, including any virus strain whose genome has been fully or partially sequenced. At least some of these sequences may be found in the NCBI nucleotide database or in the Los Alamos databases on immunodeficiency viruses. The term “derive” appearing in the definition of the polynucleotides merely specifies that the sequence of said polynucleotide may be identical to the corresponding sequence in any IV strain or HTLV strain, or may vary to the extent that it encodes polypeptides, antigens, proteins, or fragments thereof, of IV or HTLV that meet(s) the definition of the “antigen” or “polypeptide” according to the present invention. In particular, a polynucleotide derives from the nucleic acid of a IV or HTLV strain when it is codon-optimized with respect to such sequence. Accordingly, the term does not restrict the production mode of the polynucleotide. The polynucleotide may encode an antigen or a polypeptide mutated as compared to a wild-type sequence of the antigen or polypeptide, in particular within the immunosuppressive domain of ENV and NEF.

Alternatively, fragments may be short polypeptides with at least 10 amino acid residues, which harbor epitope(s) of the native protein listed in the Immune Epitope database and analysis resource (http://www.iedb.org). Fragments in this respect also include polyepitopes.

Since the transcription of the viral RNA of MeV follows a gradient from the 5′ to the 3′ end, the inventors found that cloning two polynucleotides at different locations within the cDNA encoding the full-length antigenomic (+) RNA of the measles virus may lead to the production of higher yield of antigenic particles and/or SIV, HIV or HTLV virus like particles (VLPs), while this production may be less important when the polynucleotides are all cloned within a single and same location. Furthermore, cloning the heterologous polynucleotides at different locations may reduce the attenuation of the expression of the encoded polypeptides. Indeed, when several genes are cloned within a single ATU, it may lead to reduction of the expression of the encoded polypeptides. Particular nucleic acid constructs according to this embodiment are illustrated in FIG. 1 and FIG. 20 and in the examples. Accordingly, as an alternative embodiment, several polynucleotides may be inserted within a single ATU, leading for example to the expression of a fusion protein comprising a plurality of antigens encoded by the several polynucleotides.

The cDNA molecule encoding the full-length antigenomic (+) RNA strand of the MeV may be characteristic of or may be obtained from an attenuated strain of MeV. An “attenuated strain” of MeV is defined as a strain that is avirulent or less virulent than the parent strain in the same host, while maintaining immunogenicity and possibly adjuvanticity when administrated in a host for preserving immunodominant T and B cell epitopes and possibly the adjuvancity such as the induction of T cell costimulatory proteins or cytokine IL-12.

An attenuated strain of a measles virus accordingly refers to a strain which has been serially passaged on selected cells and, possibly, adapted to other cells to produce seed strains suitable for the preparation of human vaccine strains, harboring a stable genome which would not allow reversion to pathogenicity nor integration in host chromosomes. As a particular “attenuated strain”, an approved strain for a vaccine is an attenuated strain suitable for the invention when it meets the criteria defined by the FDA (US Food and Drug Administration); i.e. it meets safety, efficacy, quality and reproducibility criteria, after rigorous reviews of laboratory and clinical data (www.fda.gov/cber/vaccine/vacappr.htm).

In particular, the cDNA molecule encoding the full-length antigenomic (+) RNA strand of the MeV is obtained from an attenuated virus strain selected from the group comprising of consisting of the Schwarz strain, the Zagreb strain, the AIK-C strain, the Moraten strain, the Philips strain, the Beckenham 4A strain, the Beckenham 16 strain, the Edmonston seed A strain, the Edmonston seed B strain, the CAM-70 strain, the TD 97 strain, the Leningrad-16 strain, the Shanghai 191 strain and the Belgrade strain. All these strains have been described in the prior art. The invention uses in particular strains that have been allowed for use as commercial vaccines. In particular, the cDNA molecule encoding the full length antigenomic (+) RNA strand of the MeV is obtained from the Schwarz strain.

According to a particular embodiment of the invention, the cDNA molecule is placed under the control of heterologous expression control sequences.

The insertion of such a control for the expression of the cDNA, is favorable when the expression of this cDNA is sought in cell types which do not enable full transcription of the cDNA with its native control sequences.

According to a particular embodiment of the invention, the heterologous expression control sequence comprises the T7 promoter and T7 terminator sequences. These sequences are respectively located 5′ and 3′ of the coding sequence for the full length antigenomic (+) RNA strand of MeV and from the adjacent sequences around this coding sequence.

In a particular embodiment of the invention, the cDNA molecule, which is defined here above is modified, i.e. comprises additional nucleotide sequences or motifs.

In a preferred embodiment, the cDNA molecule used according to the invention further comprises, at its 5′-end, adjacent to the first nucleotide of the nucleotide sequence encoding the full-length antigenomic (+) RNA strand of the MeV approved vaccine strain, a GGG motif followed by a hammerhead ribozyme sequence and comprises, at its 3′-end, adjacent to the last nucleotide of said nucleotide sequence encoding the full-length anti-genomic (+) RNA strand, the sequence of a ribozyme. The Hepatitis delta virus ribozyme (5) is appropriate to carry out this preferred embodiment.

The GGG motif placed at the 5′ end, adjacent to the first nucleotide of the above coding sequence improves the efficiency of the transcription of said cDNA coding sequence. As a requirement for the proper assembly of measles virus particles is the fact that the cDNA encoding the antigenomic (+) RNA complies with the rule of six, when the GGG motif is added, a ribozyme is also added at the 5′ end of the coding sequence of the cDNA, 3′ from the GGG motif, in order to enable cleavage of the transcript at the first coding nucleotide of the full-length antigenomic (+) RNA strand of MeV.

In order to prepare the nucleic acid construct of the invention, the preparation of a cDNA molecule encoding the full-length antigenomic (+) RNA of a measles virus disclosed in the prior art is achieved by known methods. The obtained cDNA provides especially the basis for the genome vector involved in the rescue of recombinant measles virus particles when it is inserted in a vector such as a plasmid.

A particular cDNA molecule suitable for the preparation of the nucleic acid construct of the invention is the one obtained using the Schwarz strain of measles virus. Plasmid pTM-MVSchw, in particular of SEQ ID No: 25, which contains an infectious MeV cDNA corresponding to the anti-genome of the Schwarz MV vaccine strain and is used for preparation of recombinant vectors encompassing the heterologous polynucleotides of the invention, has been described elsewhere (21). Accordingly, the cDNA used within the present invention may be obtained as disclosed in WO2004/000876 or may be obtained from plasmid pTM-MVSchw deposited by Institut Pasteur at the CNCM under No 1-2889 on Jun. 12, 2002, the sequence of which is disclosed in WO2004/000876 incorporated herein by reference. The plasmid pTM-MVSchw has been obtained from a Bluescript plasmid and comprises the polynucleotide coding for the full-length measles virus (+) RNA strand of the Schwarz strain placed under the control of the promoter of the T7 RNA polymerase. It has 18967 nucleotides and a sequence represented as SEQ ID NO: 25. cDNA molecules (also designated cDNA of the measles virus or MeV cDNA for convenience) from other MeV strains may be similarly obtained starting from the nucleic acid purified from viral particles of attenuated MeV such as those described herein. An additional transcription unit may be a multiple-cloning site cassette previously inserted in the vector, as explained in Combredet et al. (21). An ATU may comprise cis-acting sequences necessary for the transcription of the inserted IV or HTLV genes. The heterologous polynucleotide(s) are cloned or inserted within additional transcription units (ATU) as defined here above.

These embodiments are particularly suitable for providing a nucleic acid construct suitable to treat a disease related to an immunosuppressive virus, in particular a disease associated with a HIV or HTLV infection and associated diseases.

In a particular embodiment, which may be combined with any one of the embodiments already disclosed, the third polynucleotide encodes a NEF antigen further comprising a peptide signal towards its 5′ end, allowing the cellular export and lack of myristoylation of NEF. Such a NEF antigen may correspond to the amino acid sequence set forth in SEQ ID No: 22 or SEQ ID No: 23. The peptide signal may be selected among a group of peptide signal allowing the cellular export of the NEF antigen, like but not limited to the murine IgG kappa or the human IL-2 signal sequence. These peptide signals may correspond to the amino acid residues of sequence SEQ ID No: 36 and SEQ ID No: 38 respectively, and may be encoded by polynucleotide residues of SEQ ID No: 37 and SEQ ID No: 39 respectively. To avoid myristoylation of NEF, the amino acid sequence of wild type NEF may be further mutated. A NEF antigen without myristoylation does not down-regulate the expression of CD4 and MHC-I. Mutated NEF antigen without myristoylation correspond for example to the amino acid residue sequences of SEQ ID No: 22 and SEQ ID No: 23. As another example, myristoylation is avoided for the antigen having the sequence of amino acid residues of SEQ ID No: 26, or SEQ ID No: 27, or SEQ ID No: 28, or SEQ ID No: 29, SEQ ID No: 30 or SEQ ID No: 31.

In another particular embodiment, the HBZ antigen may further comprise an outer cell attachment region, for example a GPI anchor (GPI for Glycosylphosphatidylinositol). Such an embodiment allows the expression of HBZ, its cellular exportation towards the cell membrane, and its localization anchored on the outer surface of the cell membrane. GPI is a short glycolipid. It may be encoded to be attached to the 3′ end of the HBZ antigen or to the 3′ end of the HBZ-TAX antigen after translation of the heterologous polynucleotide encoding the HBZ-TAX antigen, as illustrated in SEQ ID No: 51. The GPI anchor may correspond to the amino acid residues sequence of SEQ ID No: 34, encoded by the nucleotide sequence of SEQ ID No: 35. HBZ may also be encoded by a polynucleotide further comprising a peptide signal, in particular towards its 5′ end, for example for allowing the cellular export and lack of myristoylation of HBZ. The single peptide may be selected among a group of peptide signal allowing the cellular export of the HBZ antigen, like but not limited to the murine IgG kappa or the human IL-2 signal sequence. These peptide signals may correspond to the amino acid residues of sequence SEQ ID No: 36 and SEQ ID No: 38 respectively, and may be encoded by polynucleotide residues of SEQ ID No: 37 and SEQ ID No: 39 respectively.

In another particular embodiment, the NEF antigen may further comprise an outer cell attachment region, for example a GPI anchor (GPI for Glycosylphosphatidylinositol). Such an embodiment allows the expression of NEF, its cellular exportation towards the cell membrane, and its localization anchored on the outer surface of the cell membrane. GPI is a short glycolipid. It may be encoded to be attached to the 3′ end of the NEF antigen after translation of the heterologous polynucleotide encoding the NEF antigen, as illustrated in SEQ ID No: 30 and SEQ ID No: 31. The GPI anchor may correspond to the amino acid residues sequence of SEQ ID No: 34, encoded by the nucleotide sequence of SEQ ID No: 35.

According to a preferred embodiment, the invention also concerns modification and in particular optimization of the polynucleotides to allow an efficient expression of the HIV, SIV or HTLV antigens, proteins, polypeptides, or fragments thereof, in a host cell.

Accordingly, optimization of the polynucleotide sequence can be operated avoiding cis-active domains of nucleic acid molecules: internal TATA-boxes, chi-sites and ribosomal entry sites; AT-rich or GC-rich sequence stretches; ARE, INS, CRS sequence elements; repeat sequences and RNA secondary structures; cryptic splice donor and acceptor sites, branch points.

The optimized polynucleotides may also be codon optimized for expression in a specific cell type, in particular may be modified for the Maccaca codon usage or for the human codon usage. This optimization allows increasing the efficiency of chimeric infectious particles production in cells without impacting the amino acid composition of the expressed protein(s).

In particular, the optimization of the polynucleotide encoding the HIV, SIV or HTLV antigens may be performed by modification of the wobble position in codons without impacting the identity of the amino acid residue translated from said codon with respect to the original one.

Optimization is also performed to avoid editing-like sequences from Measles virus. The editing of transcript of measles virus is a process which occurs in particular in the transcript encoded by the P gene of measles virus. This editing, by the insertion of extra G residues at a specific site within the P transcript, gives rise to a new protein truncated compared to the P protein. Addition of only a single G residue results in the expression of the V protein, which contains a unique carboxyl terminus (24).

In the polynucleotides according to this particular embodiment of the invention, the following editing-like sequences from measles virus can be mutated: AAAGGG, AAAAGG, GGGAAA, GGGGAA, as well as their complementary sequence: TTCCCC, TTTCCC, CCTTTT, CCCCTT. For example, AAAGGG can be mutated in AAAGGC, AAAAGG can be mutated in AGAAGG or in TAAAGG or in GAAAGG, and GGGAAA in GCGAAA.

Hence, the nucleic acid construct(s) of the invention may comprise at least one of the following sequences, or a plurality of the following sequences, or at least two of the following sequences, or the three of the following sequences:
SEQ ID No: 32; SEQ ID No: 33; SEQ ID No: 40; SEQ ID No: 41; SEQ ID No: 42; SEQ ID No: 43 and/or SEQ ID No: 44; in particular at least SEQ ID No: 32 and SEQ ID No: 33, or at least SEQ ID No: 40, or SEQ ID No: 41.

In an embodiment of the invention, the first nucleic acid construct has a recombinant cDNA sequence selected from the group consisting of:
SEQ ID No: 32 (construct MeV-SIVgag-HIVenv Cons B WT);
SEQ ID No: 40 (construct MeV-SIVgag-HIVenv Cons B MT);
SEQ ID No: 33 (construct MeV-SIVgag-HIVenv SF162 WT);
SEQ ID No: 41 (construct MeV-SIVgag-HIVenv SF162 MT);
SEQ ID No: 43 (construct MeV-SIVgag-HIVenv gp41 WT); and
SEQ ID No: 44 (construct MeV-SIVgag-HIVenv gp41 MT).

In an embodiment of the invention, the second nucleic acid construct has a recombinant cDNA sequence selected from the group consisting of:
SEQ ID No: 33 (construct MeV-NEF SIV WT); and
SEQ ID No: 41 (construct MeV-NEF SIV MT).

In an embodiment of the invention, the first nucleic acid construct has the recombinant cDNA sequence of SEQ ID No: 44 (construct MeV-SIVgag-HIVenv gp41 MT) and the second nucleic acid construct has the recombinant cDNA sequence of SEQ ID No: 41 (construct MeV-NEF SIV MT).

In an embodiment of the invention, the first nucleic acid construct has the recombinant cDNA sequence of SEQ ID No: 40 (construct MeV-SIVgag-HIVenv Cons B MT) and the second nucleic acid construct has the recombinant cDNA sequence of SEQ ID No: 41 (construct MeV-NEF SIV MT).

In an embodiment of the invention, the first nucleic acid construct has the recombinant cDNA sequence of SEQ ID No: 41 (construct MeV-SIVgag-HIVenv SF162 MT) and the second nucleic acid construct has the recombinant cDNA sequence of SEQ ID No: 41 (construct MeV-NEF SIV MT).

According to any one of the particular embodiments of the invention, it is provided nucleic acid constructs comprising polynucleotide(s) which increase the efficiency of chimeric recombinant MeV-HIV, MeV-SIV or MeV-HTLV infectious particles production.

Alternatively, or complementarily, the heterologous polynucleotide(s) may encode any one of the following antigens, or an antigenic fragment thereof, or at least two of the following antigens, or the three of the following antigens:
the GAG of SEQ ID No: 2, SEQ ID No: 3, SEQ ID No: 4 or SEQ ID No: 5, or an antigenic fragment thereof; and/or
the ENV of SEQ ID No: 8, SEQ ID No: 10, SEQ ID No: 11, SEQ ID No: 12, SEQ ID No: 13; SEQ ID No: 20, or SEQ ID No: 21, or an antigenic fragment thereof; and/or
the NEF antigen of SEQ ID No: 15, SEQ ID No: 17, SEQ ID No: 18, SEQ ID No: 19, SEQ ID No: 22 or SEQ ID No: 23 or an antigenic fragment thereof.

In an embodiment of the invention, the nucleic acid construct has the recombinant cDNA sequence of SEQ ID No: 54 (construct MeV-HTLVgag-HTLVenv).

According to any one of the particular embodiments of the invention, it is provided nucleic acid constructs comprising polynucleotide(s) which increase the efficiency of chimeric recombinant MeV-HTLV infectious particles production.

Alternatively, or complementarily, the heterologous polynucleotide(s) may encode any one of the following antigens, or an antigenic fragment thereof, or at least two of the following antigens, or the three of the following antigens:
the GAG of SEQ ID No: 45 or SEQ ID No: 46, or an antigenic fragment thereof; and/or
the ENV of SEQ ID No: 47, SEQ ID No: 48, SEQ ID No: 52 or SEQ ID No: 53, or an antigenic fragment thereof; more particularly ENV of SEQ ID No. 48 or SEQ ID No. 53 or antigenic fragment thereof, and/or
the HBZ antigen of SEQ ID No: 49, in particular combined with SEQ ID No: 50 (corresponding to TAX) or an antigenic fragment thereof.

It should be noted that the polynucleotide(s) may encode a polypeptide as defined here above a single time or that a fragment of a polypeptide may be encoded several times by a single polynucleotide. In a preferred embodiment, each polypeptide is encoded a single time within a single heterologous polynucleotide, and more preferentially, each polypeptide is encoded a single time within the plurality of polypeptides. According to a particular embodiment of the invention, several polynucleotides wherein each polynucleotide encodes at least one HTLV antigen are combined or fused to form a polynucleotide encoding several proteins of the HTLV. These polynucleotides may distinguish from each other by the fact that they code for proteins of various strains of HTLV or for different proteins of a HTLV strains.

It should be noted that the polynucleotide(s) may encode a polypeptide as defined here above a single time or that a fragment of a polypeptide may be encoded several times by a single polynucleotide. In a preferred embodiment, each polypeptide is encoded a single time within a single heterologous polynucleotide, and more preferentially, each polypeptide is encoded a single time within the plurality of polypeptides. According to a particular embodiment of the invention, several polynucleotides wherein each polynucleotide encodes at least one HIV, SIV or HTLV antigens are combined or fused to form a polynucleotide encoding several proteins of the HIV, SIV or HTLV. These polynucleotides may distinguish from each other by the fact that they code for proteins of various strains of the HIV, SIV or HTLV or for different proteins of a HIV, SIV and HTLV strains.

In a particular embodiment of the invention, the nucleic acid construct comprises from the 5′ to 3′ end the following polynucleotides:
(a) the third heterologous polynucleotide encoding at least a NEF or HBZ (and TAX when applicable) antigen, or an immunogenic fragment thereof, wherein the third polynucleotide is operatively cloned within an ATU located upstream the N gene of the MeV, in particular within the ATU1;
(b) a polynucleotide encoding the N protein of the MeV;
(c) a polynucleotide encoding the P protein of the MeV;
(d) the first heterologous polynucleotide encoding at least a GAG antigen, or an immunogenic fragment thereof wherein the first polynucleotide is in particular operatively cloned within an ATU, in particular ATU2;
(e) a polynucleotide encoding the M protein of the MeV;
(f) a polynucleotide encoding the F protein of the MeV;
(g) a polynucleotide encoding the H protein of the MeV;
(h) the second heterologous polynucleotide encoding at least a ENV antigen, or an immunogenic fragment thereof wherein the second polynucleotide is in particular operatively cloned within an ATU, in particular ATU3;
(i) a polynucleotide encoding the L protein of the MeV, and wherein said polynucleotides are operatively linked within the nucleic acid construct and under the control of a viral replication and transcriptional regulatory elements such as MeV leader and trailer sequence(s).

Several examples of this embodiment are schematically illustrated on FIG. 1 and FIG. 20.

The various terms used therein have the same meaning as the one used in the previous particular embodiments. The different polynucleotides inserted within the nucleic acid construct encode GAG, ENV and NEF or HBZ (and TAX when applicable) antigens, or their respective immunogenic fragments or mutated versions, all originate from the same virus type, in particular the same virus strain, more particularly from HIV-1 or HTLV-1. In this construct, the ENV, GAG and NEF or HBZ antigens are especially originating from HIV in particular HIV-1 or HIV-2 or HTLV-1 or HTLV-2 or HTLV-3, and correspond to the amino acid sequences illustrated therein.

The expressions “N protein”, “P protein”, “M protein”, “F protein”, “H protein” and “L protein” refer respectively to the nucleoprotein (N), the phosphoprotein (P), the matrix protein (M), the fusion protein (F), the hemagglutinin protein (H) and the RNA polymerase large protein (L) of a measles virus and encompass reference to the respective polypeptides or antigenic fragments thereof. These components have been identified in the prior art and are especially disclosed in Fields, Virology (10).

In a particular embodiment of the invention, the nucleic acid construct comprises a recombinant cDNA whose sequence is selected from the group consisting of:
SEQ ID No: 32, SEQ ID No: 40, SEQ ID No: 43 or SEQ ID No: 44, in particular SEQ ID No: 40 or SEQ ID No: 44 (MeV-GAG-ENV);
SEQ ID No: 33 or SEQ ID No: 41, in particular SEQ ID No: 41 (MeV-NEF). wherein said sequences are described as follows:

SEQ ID No: 32 and SEQ ID No: 40

SEQ ID No: 32 and SEQ ID No: 40 are the sequences of a nucleic acid constructs according to a particular embodiment of the invention wherein said constructs contain the pTM-MVSchwarz vector wherein the sequence encoding the GAG and ENV protein of HIV-1 has been respectively cloned within the Additional Transcription Unit 2 and 3. ENV has a mutated ISD as described here above.

SEQ ID No: 33 and SEQ ID No: 41

SEQ ID No: 33 and SEQ ID No: 41 are the sequences of a nucleic acid constructs according to a particular embodiment of the invention wherein said constructs contain the pTM-MVSchwarz vector wherein the sequence encoding the NEF protein of HIV-1 has been cloned within the Additional Transcription Unit 1. NEF has a mutated ISD as described here above.

The invention also relates to a transfer vector, which may be used for the preparation of recombinant MeV-IV or MeV-HTLV particles when rescued from helper cells or production cells. Several transfer vectors are illustrated on FIG. 1 and FIG. 20. In a preferred embodiment of the invention, the transfer vector is a transfer vector plasmid suitable for the transfection of helper cells or of production cells, and comprising the nucleic acid construct according to the invention. The transfer vector plasmid may be obtained from a Bluescript plasmid and may be obtained by cloning the heterologous polynucleotide(s) of the invention in the pTM-MVSchw plasmid described here above.

In a particular embodiment of the invention, the heterologous polynucleotide encoding the NEF or HBZ antigen is located in ATU 1, the heterologous polynucleotide encoding the GAG antigen is located in ATU 2 and the heterologous polynucleotide encoding the ENV antigen is located in ATU 3, as illustrated in FIG. 1 (for HIV) and FIG. 20 (for HTLV).

In another particular embodiment, the heterologous polynucleotide encoding the GAG antigen is located in a ATU located between the P gene and the M gene of the Measles virus, the heterologous polynucleotide encoding the ENV antigen is located in a ATU located between the H gene and the L gene of the Measles virus, and, in another Measles virus, the heterologous polynucleotide encoding the NEF antigen is located in an ATU located upstream the N gene of the Measles virus (illustrated on FIG. 16A and FIG. 16B respectively). Similar construction can be made with the antigens issued from HTVL, for example illustrated on FIG. 20 and FIG. 21. As an example, the heterologous polynucleotide encoding the GAG antigen is located in a ATU located between the P gene and the M gene of the Measles virus, the heterologous polynucleotide encoding the ENV antigen is located in a ATU located between the H gene and the L gene of the Measles virus and, in another Measles virus, the heterologous polynucleotide encoding the HBZ antigen is located between the P gene and the M gene of the Measles virus.

FIG. 1 (MeV-IV) and FIG. 20 (MeV-HTLV) represents different nucleic acid constructions encompassed by the present invention. As illustrated, different combinations of antigens encoded within the nucleic acid constructs are contemplated, and the localization of the heterologous polynucleotide encoding each antigen is variable. These schemes are for illustrative purposes only. They do not limit the localization of the heterologous polynucleotides within the nucleic acid construct of the invention, and do not represent all combinations of antigens encoded by the nucleic acid construct of the invention.

In a particular embodiment, the invention concerns a nucleic acid construct which comprises a cDNA molecule encoding a full length antigenomic (+) RNA strand of a measles virus (MeV); and
(i) a first heterologous polynucleotide encoding at least one GAG antigen, a fragment thereof, or a mutated version thereof of a Human T-lymphotropic virus (HTLV), in particular GAG of SEQ ID No: 45 or SEQ ID No. 46, wherein the first heterologous polynucleotide is operatively cloned within an additional transcription unit (ATU) inserted within the cDNA of the antigenomic (+) RNA, in particular an ATU located between the P gene and the M gene of the MeV, in particular in the ATU2 inserted between the P gene and the M gene of the MeV;
(ii) a second heterologous polynucleotide encoding at least one ENV antigen, or a fragment thereof comprising an immunosuppressive domain (ISD), in particular at least one fragment comprising the transmembrane subunit of the ENV antigen, wherein the ENV antigen or its fragment is mutated within its immunosuppressive domain (ISD) and is of a Human T-lymphotropic virus (HTLV), in particular ENV of SEQ ID No: 48, wherein the second heterologous polynucleotide is operatively cloned within the same or a different additional transcription unit (ATU) as in (i) inserted within the cDNA of the antigenomic (+) RNA, in particular an ATU located between the H gene and the L gene of the MeV, in particular in the ATU3 inserted between the H gene and the L gene of the MeV;
wherein the mutation within the ISD domain of ENV reduces the immunosuppressive index of the ENV antigen; and wherein the GAG and ENV antigens, or their respective immunogenic fragments or mutated versions thereof, all originate from the same virus type, in particular are from the same virus strain, more particularly from HTLV-1.

In a particular embodiment, the invention concerns a nucleic acid construct which comprises a cDNA molecule encoding a full length antigenomic (+) RNA strand of a measles virus (MeV); and
(iii) a third heterologous polynucleotide encoding at least one HBZ antigen, a fragment thereof, or a mutated version thereof at the oncogenic domain of HBZ of a Human T-lymphotropic virus (HTLV), in particular HBZ of SEQ ID No: 49, the third heterologous polynucleotide further encoding at least one TAX antigen, a fragment thereof, a mutated version thereof at the oncogenic domain of a Human T-lymphotropic virus (HTLV), in particular TAX of SEQ ID No: 50, wherein the third heterologous polynucleotide is operatively cloned within the same or a different additional transcription unit (ATU) as in (i) or (ii) inserted within the cDNA of the antigenomic (+) RNA, in particular an ATU located between the P gene and the M gene of the MeV, in particular in the ATU2 inserted between the P gene and the M gene of the MeV.

In a particular embodiment, the invention concerns a nucleic acid construct which comprises a cDNA molecule encoding a full length antigenomic (+) RNA strand of a measles virus (MeV); and
(i) a first heterologous polynucleotide encoding at least one GAG antigen, a fragment thereof, or a mutated version thereof of a Human T-lymphotropic virus (HTLV), in particular GAG of SEQ ID No: 45 or SEQ Id No. 46, wherein the first heterologous polynucleotide is operatively cloned within an additional transcription unit (ATU) inserted within the cDNA of the antigenomic (+) RNA, in particular an ATU located between the P gene and the M gene of the MeV, in particular in the ATU2 inserted between the P gene and the M gene of the MeV;
(ii) a second heterologous polynucleotide encoding at least one ENV antigen mutated within its immunosuppressive domain (ISD) of Human T-lymphotropic virus (HTLV), in particular ENV of SEQ ID No: 48, wherein the second heterologous polynucleotide is operatively cloned within the same or a different additional transcription unit (ATU) as in (i) inserted within the cDNA of the antigenomic (+) RNA, in particular an ATU located between the H gene and the L gene of the MeV, in particular in the ATU3 inserted between the H gene and the L gene of the MeV;
(iii) a third heterologous polynucleotide encoding at least one HBZ antigen, a fragment thereof, or a mutated version thereof at the oncogenic domain of HBZ of a Human T-lymphotropic virus (HTLV), in particular HBZ of SEQ ID No: 49, wherein the third heterologous polynucleotide is operatively cloned within the same or a different additional transcription unit (ATU) as in (i) inserted within the cDNA of the antigenomic (+) RNA, in particular an ATU located between the P gene and the M gene of the MeV, in particular in the ATU2 inserted between the P gene and the M gene of the MeV;
(iv) a fourth heterologous polynucleotide encoding at least one TAX antigen, a fragment thereof, a mutated version thereof at the oncogenic domain of a Human T-lymphotropic virus (HTLV), in particular TAX of SEQ ID No: 50, wherein the fourth heterologous polynucleotide is operatively cloned within the same or a different additional transcription unit (ATU) as in (i) inserted within the cDNA of the antigenomic (+) RNA, in particular an ATU located between the P gene and the M gene of the MeV, in particular in the ATU2 inserted between the P gene and the M gene of the MeV.

The invention also concerns the use of a transfer plasmid vector or the use of the nucleic acid construct according to the invention to transform cells suitable for the rescue of recombinant viral MeV-IV or MeV-HTLV particles, in particular to transfect or to transduce such cells respectively with plasmids or with viral vectors harboring the nucleic acid construct of the invention, said cells being selected for their capacity to express required measles virus proteins for appropriate replication, transcription and encapsidation of the recombinant genome of the virus corresponding to the nucleic acid construct of the invention in recombinant, infectious, replicative recombinant MeV-IV or MeV-HTLV particles.

The nucleic acid construct of the invention and the transfer plasmid vector are suitable and intended for the preparation of recombinant infectious replicative recombinant measles—Immunodeficiency virus (MeV-IV) or replicative recombinant measles—Human T-Lymphotropic Virus (MeV-HTLV), and accordingly said nucleic acid construct and transfer plasmid vector are intended for insertion in a transfer genome vector that as a result comprises the cDNA molecule of the measles virus, especially of the Schwarz strain, for the production of said recombinant MeV-IV virus or MeV-HTLV virus, and expression of IV or HTLV polypeptide(s), possibly as IV VLPs or HTLV VLPs. The pTM-MVSchw plasmid is suitable to prepare the transfer vector, by insertion of the heterologous polynucleotide(s) as described herein necessary for the expression of IV or HTLV polypeptide(s), protein(s), antigen(s), or antigenic fragment(s) thereof. As used herein, the term “virus-like particle” (VLP) refers to a structure that in at least one attribute resembles a virus but which has not been demonstrated to be infectious as such. Virus Like Particles in accordance with the invention do not carry genetic information encoding the proteins of the Virus Like Particles, in general, virus-like particles lack a viral genome and, therefore, are noninfectious and non-replicative. In accordance with the present invention, Virus Like Particles can be produced in large quantities and are expressed together with MeV-IV or MeV-HTLV recombinant particles.

The invention also relates to the cells or cell lines thus transformed by the transfer vector of the invention and by further polynucleotides providing helper functions and proteins. Polynucleotides are thus present in said cells, which encode proteins that include in particular the N, P and L proteins of a measles virus (i.e., native MeV proteins or functional variants thereof capable of forming ribonucleoprotein (RNP) complexes), preferably as stably expressed proteins at least for the N and P proteins functional in the transcription and replication of the recombinant viral MeV-IV or MeV-HTLV particles. The N and P proteins may be expressed in the cells from a plasmid comprising their coding sequences or may be expressed from a DNA molecule inserted in the genome of the cell. The L protein may be expressed from a different plasmid. It may be expressed transitory. The helper cell is also capable of expressing a RNA polymerase suitable to enable the synthesis of the recombinant RNA derived from the nucleic acid construct of the invention, possibly as a stably expressed RNA polymerase. The RNA polymerase may be the T7 phage polymerase or its nuclear form (nlsT7).

In an embodiment, the cDNA clone of a measles virus is from the same measles virus strain as the N protein and/or the P protein and/or the L protein. In another embodiment, the cDNA clone of a measles virus is from a different strain of virus than the N protein and/or the P protein and/or the L protein.

The cells transformed or transfected with a nucleic acid construct according to the invention are able to produce recombinant measles viruses and/or IV VLPs or HTLV VLPs. Accordingly, the recombinant measles virus comprises in its genome the nucleic acid construct of the invention and is able to express at least one polypeptide, protein or antigenic fragment thereof, of the IV or HTLV. Hence, the measles virus of the invention is able to express the mutated ENV antigen, the mutated ENV protein, or the mutated ENV polypeptide, or an antigenic fragment thereof; and/or the GAG antigen, the GAG protein, or the GAG polypeptide, or an antigenic fragment thereof; and/or the mutated NEF or HBZ antigen, the mutated NEF or HBZ protein, or the mutated NEF or HBZ polypeptide, or an antigenic fragment thereof.

In a preferred embodiment of the invention, the recombinant measles virus expresses at least the mutated ENV antigen and the GAG antigen of the IV or HTLV. In another preferred embodiment of the invention, the recombinant measles virus expresses the mutated ENV antigen, the GAG antigen and the NEF or HBZ antigen of the IV or HTLV, in particular of the HIV-1 or HIV-2 or HTLV-1 or HTLV-2 or HTLV-3.

Furthermore, according to some embodiments of the invention, the recombinant measles virus also expresses at least one polypeptide or protein, or an antigenic fragment thereof, of the measles virus. In other words, the recombinant measles virus expresses at least one of the following polypeptides: the N protein, the P protein, the M protein, the F protein, the H protein and the L protein of the MeV.

According to this embodiment, the recombinant virus expresses recombinant antigenic particles of the measles virus and the IV virus or HTLV virus, allowing the elicitation of cellular response, or a humoral response, or a cellular and humoral response against polypeptides of the IV or HTLV and against polypeptides of the MeV. In particular embodiments of the invention, the elicitation of the cellular response comprises elicitation of a T cell response, in particular CD4+ and/or CD8+ T cells response, and more particularly IFNγ and IL-2.

The invention thus relates to a process for the preparation of recombinant infectious measles virus particles comprising:
(a) transfecting cells, in particular helper cells, in particular HEK293 helper cells, stably expressing T7 RNA polymerase and measles N and P proteins with the nucleic acid construct according to the invention or with the transfer plasmid vector according to the invention;
(b) maintaining the transfected cells in conditions suitable for the production of recombinant measles virus and/or IV VLPs or HTLV VLPs;
(c) infecting cells enabling propagation of the recombinant measles virus and/or the IV VLPs or HTLV VLPs by co-cultivating them with the transfected cells of step (b);
(d) harvesting the recombinant measles virus expressing at least one IV antigen or HTLV antigen, preferentially at least the ENV and the GAG antigens and optionally the NEF or HBZ antigen, the GAG antigen and the ENV antigen, preferentially issued or derived from HIV, in particular from HIV-1 or HIV-2, or derived from HTLV, in particular from HTLV-1 or HTLV-2 or HTLV-3.

According to a particular embodiment, the invention relates to a process for the preparation of recombinant infectious measles virus particles comprising:
a) transferring, in particular transfecting, the nucleic acid construct of the invention or the transfer vector containing such nucleic acid construct in a helper cell line which also expresses proteins necessary for transcription, replication and encapsidation of the antigenomic (+) RNA sequence of MeV from its cDNA and under conditions enabling viral particles assembly and
b) recovering the recombinant infectious MeV-IV or MeV-HTLV virus expressing at least one antigen, polypeptide or protein of IV or HTLV, or an antigenic fragment thereof, preferentially from HIV, more particularly from HIV-1 or from HIV-2, or from HTLV, more particularly from HTLV-1 or HTLV-2 or HTLV-3.

According to a particular embodiment of the invention, the process comprises:
a) transfecting helper cells with a nucleic acid construct according to the invention with a transfer plasmid vector, wherein said helper cells are capable of expressing helper functions to express an RNA polymerase, and to express the N, P and L proteins of a MeV virus;
b) co-cultivating said transfected helper cells of step 1) with passaged cells suitable for the passage of the MeV attenuated strain from which the cDNA originates;
c) recovering the recombinant infectious MeV-IV or MeV-HLTV virus expressing at least one polypeptide of the IV or HTLV respectively.

According to another particular embodiment of the invention the method for the production of recombinant infectious MeV-IV or MeV-HTLV comprises:
a) recombining a cell or a culture of cells stably producing a RNA polymerase, the nucleoprotein (N) of a measles virus and the polymerase cofactor phosphoprotein (P) of a measles virus, with a nucleic acid construct of the invention and with a vector comprising a nucleic acid encoding a RNA polymerase large protein (L) of a measles virus, and
b) recovering the infectious, MeV-IV or MeV-HTLV virus from said recombinant cell or culture of recombinant cells.

According to a particular embodiment of the process, recombinant MeV are produced, which express IV or HTLV protein(s) comprising at least the ENV protein and GAG protein, and/or IV VLPs or HTLV VLPs comprising at least the ENV protein and the GAG protein, and wherein the recombinant MeV and/or VLPs may express at least one other IV or HTLV proteins, or antigen, or an antigenic fragment thereof, i.e. NEF or HBZ or a fragment thereof. In other embodiment, the IV or HTLV VLPs comprise the mutated ENV protein or a fragment thereof, the GAG protein or a fragment thereof and the mutated NEF or HBZ protein or a fragment thereof. As an illustration, a process to rescue recombinant MeV expressing IV or HTLV proteins, in particular Iv or HTLV VLPs comprises the steps of:
1) cotransfecting helper cells, in particular HEK293 helper cells, that stably express T7 RNA polymerase, and measles N and P proteins with (i) a transfer vector, in particular a plasmid, comprising cDNA encoding the full-length antigenomic (+) RNA of a measles virus recombined with at least one polynucleotide encoding at least one IV or HTLV protein, for example encoding the mutated ENV protein, the GAG protein, and the mutated NEF or HBZ protein, said polynucleotides being localized within the same nucleic acid construct, or within different nucleic acid constructs, and with (ii) a vector, especially a plasmid, encoding the MeV L polymerase cDNA;
2) cultivating said cotransfected helper cells in conditions enabling the production of MeV-IV or MeV-HTLV recombinant virus;
3) propagating the thus produced recombinant virus by co-cultivating said helper cells of step 2) with cells enabling said propagation such as Vero cells;
4) recovering replicating MeV-IV or MeV-HTLV recombinant virus and IV or HTLV protein(s), in particular IV or HTLV Virus Like Particles, more particularly HIV-1 or HIV-2 or HTLV-1 or HTLV-2 or HTLV-3 protein(s), and still more particularly HIV-1 or HTLV-1 Virus Like Particles.

As used herein, “recombining” means introducing at least one polynucleotide into a cell, for example under the form of a vector, said polynucleotide integrating (entirely or partially) or not integrating into the cell. According to a particular embodiment, recombination can be obtained with a first polynucleotide, which is the nucleic acid construct of the invention. Recombination can, also or alternatively, encompasses introducing a polynucleotide, which is a vector encoding a RNA polymerase large protein (L) of a measles virus, whose definition, nature and stability of expression has been described herein.

In accordance with the invention, the cell or cell lines or a culture of cells stably producing a RNA polymerase, a nucleoprotein (N) of a measles virus and a polymerase cofactor phosphoprotein (P) of a measles virus is a cell or cell line as defined in the present specification or a culture of cells as defined in the present specification, i.e., are also recombinant cells to the extent that they have been transformed by the introduction of one or more polynucleotides as defined above. In a particular embodiment of the invention, the cell or cell line or culture of cells, stably producing the RNA polymerase, the N and P proteins, does not produce the L protein of a measles virus or does not stably produce the L protein of a measles virus, e.g., enabling its transitory expression or production. The production of recombinant MeV-IV or MeV-HTLV virus of the invention may involve a transfer of cells transformed as described herein. “Transfer” as used herein refers to the plating of the recombinant cells onto a different type of cells, and particularly onto monolayers of a different type of cells. These latter cells are competent to sustain both the replication and the production of infectious recombinant MeV-IV or MeV-HTLV virus i.e., respectively the formation of infectious viruses inside the cell and possibly the release of these infectious viruses outside of the cells possibly with release of IV immunogenic particles and/or IV VLPs, or HTLV immunogenic particles and/or HTLV VLPs. This transfer results in the co-culture of the recombinant cells of the invention with competent cells as defined in the previous sentence. The above transfer may be an additional, i.e., optional, step when the recombinant cells are not efficient virus-producing culture i.e., when infectious recombinant MeV-IV virus or MeV-HTLV virus cannot be efficiently recovered from these recombinant cells. This step is introduced after further recombination of the recombinant cells of the invention with any nucleic acid construct of the invention, and optionally a vector comprising a nucleic acid encoding a RNA polymerase large protein (L) of a measles virus.

In a particular embodiment of the invention, a transfer step is required since the recombinant cells, usually chosen for their capacity to be easily recombined are not efficient enough in the sustaining and production of recombinant infectious MeV-IV virus or MeV-HTLV. In said embodiment, the cell or cell line or culture of cells of step 1) of the above-defined methods is a recombinant cell or cell line or culture of recombinant cells according to the invention.

Cells suitable for the preparation of the recombinant cells of the invention are prokaryotic or eukaryotic cells, particularly animal or plant cells, and more particularly mammalian cells such as human cells or non-human mammalian cells or avian cells or yeast cells. In a particular embodiment, cells, before recombination of its genome, are isolated from either a primary culture or a cell line. Cells of the invention may be dividing or non-dividing cells.

According to a preferred embodiment, helper cells are derived from human embryonic kidney cell line 293, which cell line 293 is deposited with the ATCC under No. CRL-1573. Particular cell line 293 is the cell line disclosed in WO2008/078198 and referred to in the following examples as 293T7-NP. Thus, the invention also relates to a host cell, in particular an avian cell or a mammalian cell, transfected or transformed with the nucleic acid construct according to any embodiment of the invention, or transfected with a transfer plasmid vector. Suitable cells are the VERO NK or E6 cells (African green monkey kidney cells), and MRC5 cells (Medical Research Council cell strain 5). According to another aspect of this process, the cells suitable for passage are CEF cells (chick embryo fibroblasts). CEF cells can be prepared from fertilized chicken eggs as obtained from EARL Morizeau (8 rue Moulin, 28190 Dangers, France) or from any other producer of fertilized chicken eggs.

The process which is disclosed according to the present invention is used advantageously for the production of infectious replicative recombinant MeV-IV or MeV-HTLV virus appropriate for use as immunization compositions. The invention thus relates to a composition, in particular an antigenic composition, whose active principle comprises infection replicative recombinant MeV-IV or MeV-HTLV virus rescued from the nucleic acid construct of the invention and in particular obtained by the process disclosed. The composition may be a vaccine composition for administration to a human in need thereof, especially children. Said composition may be used for the treatment against IV infection or HTLV infection. Said composition may be used for the protection against AIDS or HTLV-related disease. Thus, the composition may be an immunogenic or antigenic composition for the protective or prophylactic treatment against an HIV or HTLV infection. In particular, the active ingredients or active principles within the composition comprise recombinant MeV-HIV or HTLV particles, said recombinant MeV-HIV or MeV-HTLV particles being rescued from a transfer plasmid vector according to the invention. In a particular embodiment of the invention, the composition is a vaccine.

The invention also concerns the recombinant MeV-HIV or MeV-HTLV infectious replicating virus particles in association with HIV or HTLV polypeptide(s) or protein(s), or antigenic fragment(s) thereof, possibly associated HIV or HTLV VLPs, or any composition according to the invention, for the use in the treatment or the prevention of an infection by HIV or HTLV virus in a subject, in particular a human subject, in particular a child.

The invention also concerns recombinant MeV-HIV or MeV-HTLV infectious, replicative virus and associated HIV or HTLV polypeptide(s) or protein(s), or antigenic fragment(s) thereof, and potentially associated HIV or HTLV VLPs for use in an administration scheme and according to a dosage regime that elicits an immune response, advantageously a protective immune response, against HIV or HTLV virus infection or induced disease, in particular in a human subject, in particular a child.

In a particular embodiment of the invention, the composition or the use of the composition is able to elicit immunization of a subject, in particular a human subject, in particular a child, after a single injection, like a subcutaneous injection, in particular an intramuscular injection. In other words, the composition or the use of the composition may require a single administration of a selected dose of the recombinant MeV-HIV or MeV-HTLV infectious replicative virus. Alternatively, it may require multiple doses administration in a prime-boost regimen. Priming and boosting may be achieved with identical active ingredients consisting of recombinant MeV-HIV or MeV-HTLV infectious, replicative virus and associated HIV or HTLV polypeptide(s) and protein(s), or antigenic fragment(s) thereof, and/or HIV or HTLV VLPs. In another embodiment, the antigens encoded by the nucleic acid construct(s) may be different, or issued from different strains of a single type of virus, in priming and boosting, as illustrated in the examples of the invention.

This embodiment is particularly useful in a vaccine prime-boost administration regimen, wherein the nucleic acid construct comprising the polynucleotide encoding at least one fragment of a ENV antigen is used as a prime or as a boost to promote neutralizing antibodies and therapeutic antibodies inhibitors of the NK-dependent T CD4 cell depletion.

Thus, according to a particular embodiment of the invention, it is provided a vaccine comprising as one ingredient a MeV-HIV or a MeV-HTLV infectious, replicative virus and associated HIV or HTLV polypeptide(s) or protein(s), and/or HIV or HTLV VLPs, and/or genetic constructs according to the invention, for use in a prime/boost administration regimen, in particular prime/boost vaccination, and more particularly heterologous prime/boost vaccination. A heterologous prime/boost vaccination comprises the administration to a subject of a first dose (prime dose) comprising a first therapeutic agent, and a second dose (boost dose) comprising a second, different, therapeutic agent, the second dose being administered after the first dose (usually several weeks).

According to the invention, the heterologous prime/boost vaccination is performed with ingredient a MeV-HIV or a MeV-HTLV infectious, replicative virus and associated HIV or HTLV polypeptide(s) or protein(s), and/or HIV or HTLV VLPs, and/or genetic constructs according to the invention as a therapeutic agent for the prime dose or the boost dose. Another therapeutic agent is provided for the prime dose or the boost dose, the case being. The second therapeutic agent may be a RNA, in particular a mRNA, a messenger RNA-liposome type compound, vaccine vectors of adenovirus type expressing the vaccine HIV or HTLV antigens, non-measles vectors expressing vaccine HIV or HTLV antigens, proteins, peptides, full-length protein antigen or peptide subdomain in the form of peptides, synthetic or natural, corresponding to HIV or HTLV antigens. The antigens provided may correspond to the antigen encoded by any genetic construct disclosed herein. The efficiency of such an administration is illustrated in the examples of the invention (see FIG. 19). Protein and/or peptide prime or boost administration may consist of full-length and/or subdomain proteins of the antigens expressed during prime or boost (the case being) by the recombinant measles virus. For HIV vaccination, the protein administered may be subdomains of the ENV (i.e. ectodomain of gp41) and NEF antigens in the form of peptides and/or small proteins of approximately 50-70 amino acids. For HTLV vaccination, the protein administered may be peptides and/or small proteins from subdomains of the ENV, HBZ and/or TAX antigens.

These peptides, small proteins, and viruses used for heterologous primes or boosts may be in particular administered in the presence of adjuvants. These adjuvants can be of several types, in particular Aluminum salts (Alum), Tween (polysorbate) 80 and squalene (MF59) emulsions, TLR4 agonists such as 3-O-desacyl-4′-monophosphoryl lipid A (MPL), synthetic TLR7/8 agonists such as imidazoquinoline (R848, Resiquimod), and/or TLR9 agonists such as a 22-mer single-stranded DNA (CpG 1018).

The vaccine according to the invention may be administered by different routes. In an embodiment, the vaccine according to the invention is administered by subcutaneous, intramuscular and/or intramucosal routes. The inventors demonstrated in non-human primates the vaccine efficacy of MeV-HIV administered by subcutaneous and intranasal (mucosal) routes (FIGS. 13 and 14). In a preferred embodiment of the invention, the vaccine comprises the administration of MeV-HIV or MeV-HTLV by the intramucosal route (e.g. tongue, rectum or vagina), and more particularly by the nasal route. These types of administration aim at inducing IgA antibodies at the mucosal level in order to prevent HIV or HTLV infections by blocking the virus at its main entry sites. In an embodiment, the vaccine comprises the administration of MeV-HIV or MeV-HTLV by the intramucosal route (e.g. tongue, rectum or vagina), and more particularly by the nasal route as a prime administration, in particular in a heterologous prime/boost vaccination. In an embodiment, the vaccine comprises the administration of MeV-HIV or MeV-HTLV by the intramucosal route (e.g. tongue, rectum or vagina), and more particularly by the nasal route as a boost administration, in particular in a heterologous prime/boost vaccination.

The invention also concerns an assembly of different active ingredients including as one of these ingredients recombinant MeV-HIV or MeV-HTLV infectious, replicative virus and associated HIV or HTLV polypeptide(s) or protein(s), and/or HIV or HTLV VLPs. The assembly of active ingredients is advantageously for use in immunization of a host, in particular a human host.

In monkeys, the inventors have shown that administration of recombinant MeV-HIV infectious, replicative virus elicits an immune response and especially elicits production of neutralizing antibodies against HIV-related polypeptides. Accordingly, it has been shown that administration of the active ingredients according to the invention elicits immunization of the host. The vaccine according to the invention is safe, leads to immune answer within the host, which encompasses especially CD4+ and CD8+ T cell responses, and in particular IFNγ and/or IL-2 responses. As shown in the examples, the vaccine according to the invention induces antigen-specific T cell responses. It has also been shown that immunized monkey hosts have a reduced viremia when subjected to a SHIV infection. Administration of recombinant MeV-HTLV infectious, replicative virus may elicit an immune response and especially may elicit production of neutralizing antibodies against HIV-related polypeptides, and may be associated with similar immune answer as those observed with MeV-HIV infectious replicative virus.

The composition according to the invention may be able to elicit production of recombinant HIV or HTLV-specific immunoglobulins, especially IgM and IgG, and neutralizing antibodies. The composition according to the invention should be a safe vaccine, immunogenic and efficacious in a host. The compositions and their use may confer at least T cell response and may confer immunity against a HIV or HTLV infection in a vaccinated host.

The composition according to the invention also concerns recombinant MeV-HIV or HTLV infectious, replicative virus and associated HIV or HTLV polypeptide(s) or protein(s), or antigenic fragment(s) thereof, and potentially associated HIV or HTLV VLPs for use in an administration scheme and according to a dosage regime that elicits an immune response, advantageously a protective immune response, against measles virus infection or induced disease, in particular in a human subject, in particular a child.

The invention also concerns the recombinant MeV-HIV or MeV-HTLV infectious replicating virus particles in association with HIV or HTLV polypeptide(s) or protein(s), or antigenic fragment(s) thereof, and/or HIV or HTLV VLPs, or any composition according to the invention, for the use in the treatment or the prevention of an infection by measles virus in a subject, in particular a human subject, in particular a child.

### DESCRIPTION OF THE FIGURES

Some of the figures, to which the present application refers, are in color. The application as filed contains the color print-out of the figures, which can therefore be accessed by inspection of the file of the application at the patent office.

FIG. 1. Schematic representation of nucleic acid constructs of the invention. Different genetic sequences have been inserted in ATU1, 2 or 3 as shown in FIGS. 1A-K, with the sequences of SIV/HIV gag and HIV env (A), SIV/HIV Nef (B), HIV Nef, HIV gag and HIV env (C-F), HIVgp41 (G), HIV GAG and HIV gp41 (H).

FIG. 2. Electron microscopy image of Vero cells infected by a recombinant MeV-SIVgag-HIVenv virus. N: nucleus; C: cytosol; arrowheads: MeV viral particles; arrows: gag-forming VLPs. MOI: 0.01.

FIG. 3. Summary of vaccine and immunization schedule and repeated low-dose SHIV162P3 challenges. A: Prime and boost 1 were subcutaneous. Boost 2 was both subcutaneous and intranasal. Challenges were intra-rectal (i.r.). Subcutaneous immunizations were performed at 2 distinct points in animals left and right back, while intranasal immunization was performed with a spray vaccine. B: Vaccines and doses used for immunization of the animals. MV: MeV control; Wt: MeV-SHIV Wild-type; Mt: MeV-SHIV Mutated (mutated SIV-nef and HIV-env; wild-type SIV-gag).

FIG. 4. Infection in animals challenged with SHIV-SF162p3. A: Percentage of infected animals after each challenge according to Kaplan-Meier estimator. B: Kaplan-Meier estimation of animals infected above the threshold of 10⁴ virus copies/ml of serum. p values are calculated by the log-rank Mantel-Cox test; *:p<0.05; **:p<0.01.

FIG. 5. Peaks of SHIV RNA copies in each group of immunized and challenged animals. A: log peak after challenge. B: log peak after 1 week. MV: MeV control; Wt: MeV-SHIV Wild-type; Mt: MeV-SHIV Mutated. p values are calculated by the Kruskal-Wallis and Dunn's multiple comparisons tests. *: p<0.05; **p<0.01. Horizontal bars represent the median values.

FIG. 6. Plasma virus-load kinetic in vaccinated (Wt and Mt) and control (MV) animals presented with interquartile ranges. p values are calculated according to the Wilcoxon matched-pairs signed rank tests. *:p<0.05; **:p<0.01. MV: MeV control; Wt: MeV-SHIV Wild-type; Mt: MeV-SHIV Mutated. Horizontal bars represent the median values.

FIG. 7. Post-challenge lymphocyte NADIR count. Number of lymphocytes×1000 par μl of blood. p values are calculated by the Kruskal-Wallis and Dunn's multiple comparisons tests. *:pw0.05. MV: MeV control; Wt: MeV-SHIV Wild-type; Mt: MeV-SHIV Mutated. Horizontal bars represent the median values.

FIG. 8. Detection of SHIV162p3 by qRT-PCT of SHIV RNA copies per plasma ml measured after 13 weeks post first detection. MV: MeV control; Wt: MeV-SHIV Wild-type; Mt: MeV-SHIV Mutated.

FIG. 9. SHIV integrated DNA copies per 10⁶ cells in different cell types or organs. A: PBMCs. B: Spleen. C: axillary lymph node. D: inguinal lymph node. E: Rectum. MV: MeV control; Wt: MeV-SHIV Wild-type; Mt: MeV-SHIV Mutated. p values reflect Kruskal-Wallis and Dunn's comparisons tests. *: p<0.05; **p<0.01. Horizontal bars represent the median values.

FIG. 10. Post-prime IFNγ cellular immune response. A: IFNγ anti-GAG vaccine-induced immune responses analyzed after 2 weeks after prime. B: IFNγ anti-NEF vaccine-induced immune responses analyzed after 2 weeks after prime. Response analyzed with IFNγ Fluorospot assay (FLISPOT) and illustrated as spot forming cells (SFC) against SIV-GAG and SIV-NEF. Horizontal bars represent the median values.

FIG. 11. Post-boosts IL-2 cellular immune response. A: IL-2 anti-GAG vaccine-induced immune responses analyzed 1 week after the second boost. B: IL-2 anti-NEF vaccine-induced immune responses analyzed 2 weeks after the second boost. Measures performed by IL-2 intra-cellular staining (ICS) assays. MV: MeV control; Wt: MeV-SHIV Wild-type; Mt: MeV-SHIV Mutated (mutated SIV-nef and HIV-env; wild-type SIV-gag). p values calculated with the Kruskal-Wallis test and Dunn's multiple comparisons tests; *: p<0.05; **:p<0.01. Horizontal bars represent the median values.

FIG. 12. Log of SHIV RNA copies per ml at peak viremia. A: Post-prime anti-GAG FLISPOT at week+2. B: Post-boost anti-ENV at week+24. Results are presented as IFNγ producing spot-forming cells (SFC) per 106 PBMCs in vaccinated animals. Red triangles: Wt: MeV-SHIV Wild-type; Green triangles: Mt: MeV-SHIV Mutated (mutated SIV-nef and HIV-env; wild-type SIV-gag). Statistical analyses are performed with the non-parametric Spearman correlation, two tailed p values.

FIG. 13. Vaccine-elicited humoral immune response—antibody titration by ELISA (Log end point ELISA titers) against A: Env (gp120); B: Gag; C: Nef; D: MeV (MV) proteins. p values are calculated by the Kruskal-Wallis and Dunn's multiple comparisons tests. *:p<0.05; **:p<0.01: ***:p<0.001. Horizontal bars represent the median values. Serums were collected at base line (week-2), prime (week+2), boost (2 weeks post second boost=week+31), and post-challenge (2 weeks from first positive qRT-PCR of SHIVSF162p3 RNA).

FIG. 14. Vaccine-elicited cellular response—IFNγ producing cells specific to A: Env; B: Gag; C: Nef: D: MeV (MV) proteins. p values are calculated by the Kruskal-Wallis and Dunn's multiple comparisons tests. *:p<0.05; **:p<0.01; ***:p<0.001. Horizontal bars represent the median values. PBMCs were collected at base line (week−2), prime (week+2), boost (2 weeks post second boost=week+31), and post-challenge (2 weeks from first positive qRT-PCR of SHIVSF162p3 RNA).

FIG. 15. Eosinophil levels vs. peak viremia in vaccinated animals. Number of Eosinophils×1000 per RNA copies/ml of serum (Log 10). p values reflect Spearman correlation non-parametric tests. **:p<0.01.

FIG. 16. Schematic illustrations of nucleic acid constructs according to the invention. (A) represents a MeV vector comprising a first heterologous polynucleotide encoding a GAG antigen from SIV, and a second heterologous polynucleotide encoding a ENV antigen from HIV. (B) represents a MeV vector comprising a third heterologous polynucleotide encoding a NEF antigen from SIV.

FIG. 17. Production of SIV GAG and HIV GP41 in cells infected with MeV-SHIV GAG GP41. Western blot analysis of GP41 and GAG antigens from Vero cells infected with MeV HIV GAG ENV-gp41. Cell: cell lysat; VLP: virus-like particle. Staining: 2F5 anti-GP41 antibody; 55-2F12 anti-GAG antibody.

FIG. 18. Cellular response in mice after vaccination with Measles-SHIV virus according to the invention. Measurement by ELISPOT of cell immune responses following mouse vaccination with MeV GAG GP41 Wt or Mt (SIV GAG HIV GP41), MV GAG ENV Wt (SIV GAG HIV ENV) and MeV control virus.

FIG. 19. Comparison between heterologous prime/boost vaccination and homologous prime/boost vaccination for the induction of antibodies directed against the immunosuppressive (IS) domain of the HIV envelope. Comparison of IS domain antibody titers obtained in two groups of 6 mice vaccinated with Me-HIV in homologous prime/boost (Me-HIV/Me-HIV) or heterologous Me-HIV prime and gp41 boost peptides (Me-HIV/gp41 peptides). In contrast to the homologous prime/boost (white box-plot), the heterologous prime/boost induced high titers of antibodies directed against the IS domain (red box-plot). N=4 experiments, means and SD are shown.

FIG. 20. Schematic representation of nucleic acid constructs of the invention. Different genetic sequences have been inserted in ATU1, 2 or 3 as shown in FIG. 20A-C; HTLV GAG and HTLV ENV (A), HBZ-Tax, HTLV gag and HTLV env (B and C).

FIG. 21. Schematic illustrations of nucleic acid constructs according to the invention. It is illustrated a MeV vector comprising a first heterologous polynucleotide encoding a GAG antigen from HTLV, and a second heterologous polynucleotide encoding a ENV antigen from HTLV.

FIG. 22. HTLV GAG and ENV production in cell infected with the nucleic acid construct MeV HTLV GAG ENV of the invention. Western blot analysis of HTLV ENV (A) and GAG (B) antigens from Vero cells infected with MeV HTLV GAG ENV. Black arrow point to the blots corresponding to ENV or GAG antigen molecular weights. Cell: cell lysat; VLP: virus-like particle. Staining: VC-17 anti-HTLV ENV Antibody; 4D7-H5 anti-GAG antibody.

FIG. 23: Cellular response in mice after vaccination with a Measles-HTLV virus according to the invention. Measurement by ELISPOT of cell immune responses following mouse vaccination with MeV HTLV GAG ENV Wt or Mt, and MeV control virus.

### EXAMPLES

Materials and Methods

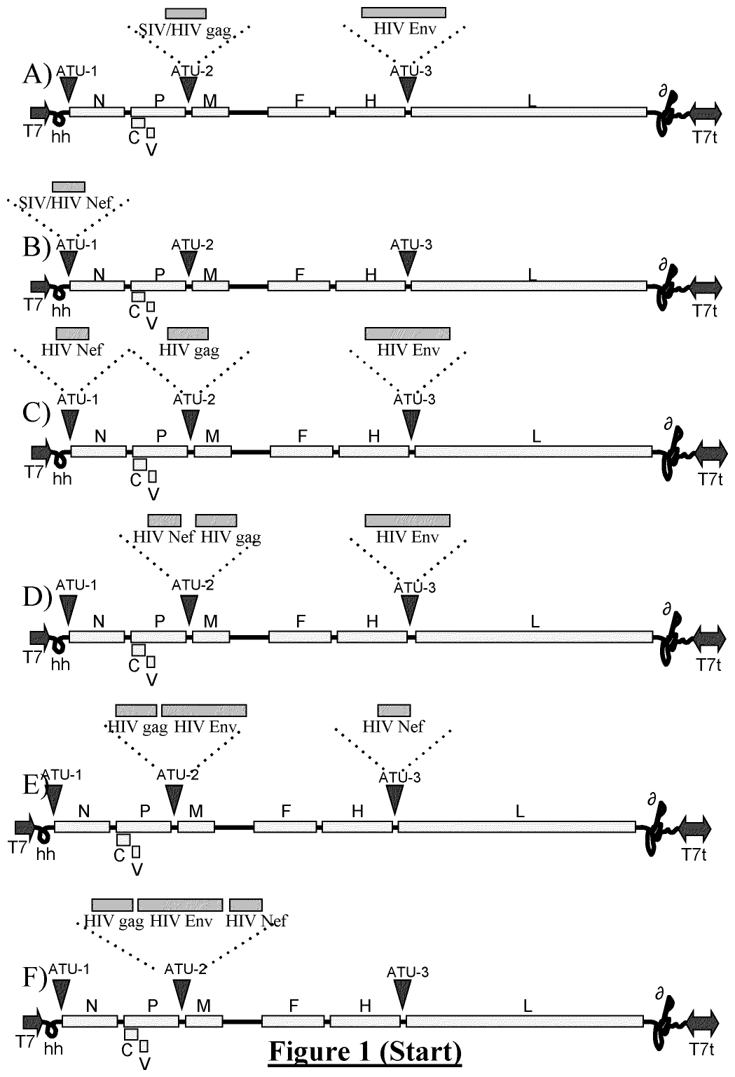

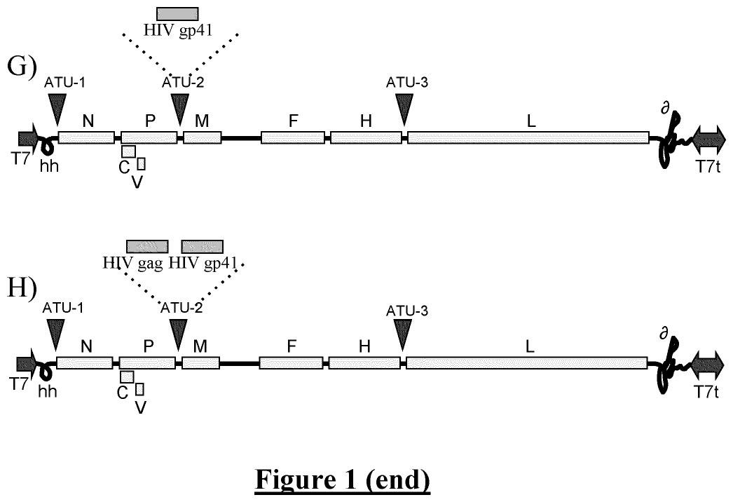

Plasmid construction and vector production. The plasmid pTM-MVSchw carries an infectious cDNA corresponding to the anti-genome of the Schwarz MV vaccine strain (9). An additional transcription unit (ATU) has been inserted into the plasmid backbone by site-directed mutagenesis between the MV P and M genes. Each MV open reading frame (ORF) expression is controlled by its own cis-acting element. The expression of additional ORFs inserted in the ATU is controlled by cis-acting elements modeled after those present in the N/P boundary region (allowing for the necessary transient transcription stop upstream of the transgene, autonomous transcription, capping and polyadenylation of the transgene). Into a single pTM-MVSchw plasmid: SIVmac239 Gag and HIV-1 Env (Consensus B Env delta V1/V2 for the prime and SF162 Env for the boosts) genes have been sub-cloned in the ATU2 and ATU3 respectively (FIG. 1). Into another pTM-MVSchw plasmid: SIVmac239 Nef gene has been sub-cloned into ATU2 (FIG. 1). SIVmac239 Nef gene encodes for a secreted and non-myristoylated form. The corresponding viruses were rescued from the pTM-MVSchw-SHIV plasmids using a helper cell-based system. Briefly, helper HEK293 cells expressing both the T7-RNA polymerase and the Schwarz MV N and P proteins (HEK293-T7-MV) were co-transfected with the pTM-MVSchw-SHIV (either encoding for Gag-Env or Nef antigens) and a plasmid expressing the Schwarz MV polymerase L. Subsequently, transfected HEK293-T7-MV helper cells were gently harvested and cocultured with MRC-5 cells for the amplification of the MVSchw-SHIV viruses. Virus titers were determined by endpoint titration on Vero cells and expressed as TCID50/ml. Into a single pTM-MVSchw plasmid, polynucleotide sequences encoding GAG of HTLV-1 of SEQ ID No: 45 and ENV of HTLV-1 of SEQ ID No: 48 have been inserted. The corresponding viruses were rescued from the pTM-MVSchw-HTLV plasmids using a helper cell-based system. Briefly, helper HEK293 cells expressing both the T7-RNA polymerase and the Schwarz MV N and P proteins (HEK293-T7-MV) were co-transfected with the pTM-MVSchw-HTLV (encoding for Gag-Env antigens) and a plasmid expressing the Schwarz MV polymerase L. Subsequently, transfected HEK293-T7-MV helper cells were gently harvested and cocultured with MRC-5 cells for the amplification of the MVSchw-HTLV viruses. Virus titers were determined by endpoint titration on Vero cells and expressed as TCID50/ml.

Transmission electron microscopy. MV-SHIV infected cells fixed in 1.6% glutaraldehyde in 0.1M phosphate buffer were collected by scraping and centrifuged. Cell pellets postfixed with 2% osmium tetroxide were dehydrated in ethanol and embedded in Epon™ 812. Ultrathin sections stained with standard uranyl acetate and lead citrate solutions were observed under a FEI Tecnai 12 electron microscope. Digital images were taken with a SIS MegaviewIII CCD camera.

Identification of HIV Env and SIV nef immunosuppressive (IS) domain mutations by tumor rejection assays. 293T cells (7.5 10⁵) were cotransfected with HIV env or nef gene fragments pointed-mutated at the IS domain inserted into pDFG retroviral vectors (1.75 μg) and expression vectors for the MLV proteins (0.55 μg for the amphotropic MLV env vector and 1.75 μg for the MLV gag and pol vector; see ref. 10). Thirty-six hours after transfection, supernatants were harvested for infection of MCA205 cells (2.5 ml per 5.10⁵ cells with 8 mg/ml polybrene). Cells were maintained in selective medium (400 units/ml hygromycin) for 3 weeks and then washed with PBS, scraped without trypsinization, and inoculated s.c. in mice flanks. Tumor area (mm²) was determined by measuring perpendicular tumor diameters, and extent of immunosuppression was quantified by an index based on tumor size (AIS domain−Aₙₒₙₑ)/Aₙₒₙₑ, where AIS domain and Aₙₒₙₑ are the mean areas at the peak of growth of tumors from mice injected with Env or Nef IS domain-expressing or control cells, respectively. Mice were maintained in the animal facility of Gustave Roussy Institute in accordance with institutional regulations.

Identification of HTLV Env immunosuppressive (IS) domain mutations by tumor rejection assays. 293T cells (7.5 10⁵) were cotransfected with HTLV env gene fragments pointed-mutated at the IS domain inserted into pDFG retroviral vectors (1.75 μg) and expression vectors for the MLV proteins (0.55 μg for the amphotropic MLV env vector and 1.75 μg for the MLV gag and pol vector; see ref. 6). Thirty-six hours after transfection, supernatants were harvested for infection of MCA205 cells (2.5 ml per 5.10⁵ cells with 8 mg/ml polybrene). Cells were maintained in selective medium (400 units/ml hygromycin) for 3 weeks and then washed with PBS, scraped without trypsinization, and inoculated s.c. in mice flanks. Tumor area (mm²) was determined by measuring perpendicular tumor diameters, and extent of immunosuppression was quantified by an index based on tumor size (AIS domain−Aₙₒₙₑ)/Aₙₒₙₑ, where AIS domain and Aₙₒₙₑ are the mean areas at the peak of growth of tumors from mice injected with Env IS domain-expressing or control cells, respectively. Mice were maintained in the animal facility of Gustave Roussy Institute in accordance with institutional regulations.

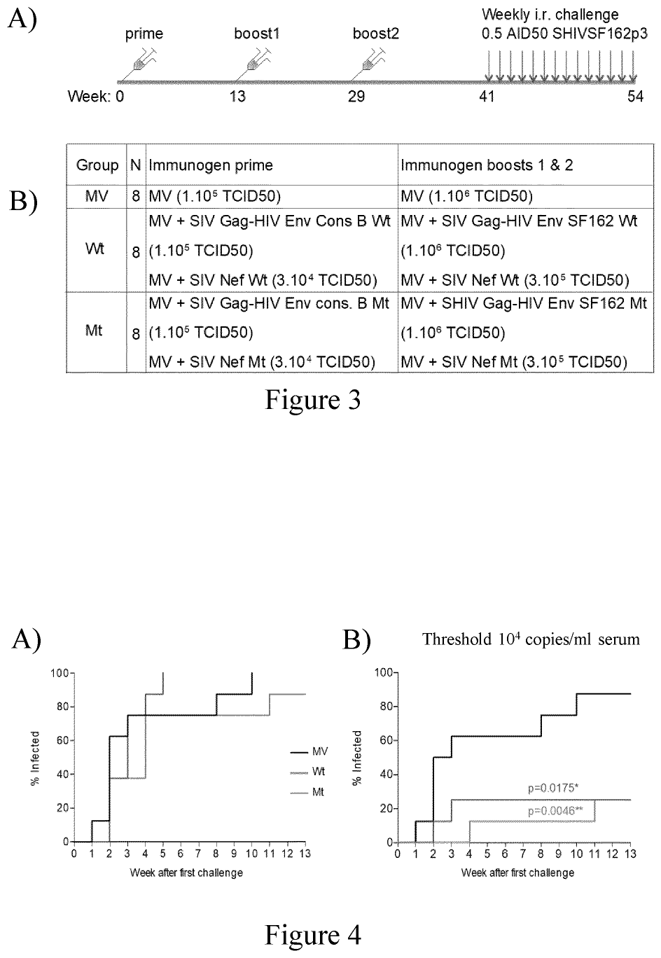

Animals, immunizations, challenge. 24 naïve male cynomolgus macaques (CM) (Macaca fascicularis), each weighing 4 to 5 kg, imported from Mauritius were assigned in the study. Animals were confirmed negative for SIV, STLV (simian T-lymphotropic virus), herpes B virus, filovirus, SRV-1, SRV-2a (Simian retrovirus 1 and 2a), and MV. Eight animals were assigned per group of immunization (FIG. 3). The three animals carrying the H6 MHC class I haplotype were equally distributed among the three experimental groups (one macaque per group) (25). (i) Group “MV”: animals were immunized with MV empty vector as a control, (ii) group “Wt”: animals were immunized with MV vector encoding for wild-type SIV Gag and Nef and wild-type HIV Env, and (iii) group “Mt”: animals were immunized MV vector encoding for wild-type SIV Gag, IS domain-Mutated SIV Nef and HIV Env. Prime was performed with wild type or ISD-mutant consensus B Env deltaV1/V2 and boost 1 and boost 2 with Wt or Mt full-length SF162 Env.

Vaccine vectors were injected subcutaneously at week 0, 13 and 29. MV, MV-SHIV WT and MV-SHIV IS domain-mutant encoding for Gag and Env proteins were injected at 1.10⁵ 50% tissue culture infective dose (TCID50), and MVSIV Wt and IS domain mutant encoding for Nef proteins at 3.10⁴ TCID50. Boost 1 and 2 were performed with a 10-fold increased dose regarding the prime (1.10⁶ TCID50 MV SHIV Gag Env Wt/Mt and 3.10⁵ TCID50 MV SIV Nef Wt/Mt). Boost 2 was administered both intranasally and subcutaneously: each animal received 1×10⁶ MVSHIV Gag Env Wt/Mt and 3×10⁵ MVSIV Nef Wt/Mt TCID50 both subcutaneously and in intra-nostril as a spray.

Macaques were repeatedly challenged once weekly by the intrarectal route with 0.5 animal infectious dose 50% (AID50) of SHIV162p3. The virus stock was provided by the NIH AIDS Research and Reference Reagent Program. Plasma viral loads were measured weekly and challenges were pursued until two consecutive qRT-PCR virus detections, with a maximum of 10 inoculations.

Plasma virus and provirus quantification. Plasma SIV RNA was quantified as previously described (26, 27). The lower limit of quantification (LOQ) and the lower limit of detection (LOD) were 37 and 12.3 copies of vRNA/mL, respectively.

Proviral DNA in PBMC and in organs was measured by quantitative PCR, using primers amplifying the gag region of SIV (30). Measurements were performed at week +13 post first SHIV detection in plasma.

FLUOROSPOT IFN-γ and IL-2 assays. IFN-γ and IL-2 responses were analyzed in PBMC by using FluoroSpot assay (FS-2122-10 Monkey IFNγ/IL2 FluoroSpot kit from Mabtech, Nacka, Sweden) according to manufacturer's instructions. The following peptide pools were used for ex vivo stimulation (2 μg/mL): Gag-SIVp15-p27 (15mers, provided by Proteogenix) in 1 pool of 85 peptides; Nef-SIV (15mers, provided by Proteogenix) in 1 pool of 63 peptides; HIV-1 Consensus B Env peptides—Complete Set (15mers, provided by NIH, cat. #9480), divided in 3 pools of 70 peptides and MV 5 Schwarz virus (1 pfu/cell). PMA/ionomycine were used as positive control. Plates were incubated for 44 h at +37° C. in an atmosphere containing 5% CO₂. Spots were counted with an automated FluoroSpot Reader ELRIFL04 (Autoimmun Diagnostika GmbH, Strassberg, Germany).

Intracellular cytokine assay (ICS). 2×10⁶ PBMCs were incubated in 200 μl of complete media (RPMI 1640 with L-glutamine containing 10% fetal calf serum FBS) with anti-CD28 (1 μg/ml) and anti-CD49d (1 μg/ml) (BD Biosciences, San Diego, CA, USA). Brefeldin A (Sigma-Aldrich, Saint-Louis, MO) was added to each well at a final concentration of 10 μg/ml and plates were incubated at 37° C., 5% CO₂ overnight and different conditions for stimulation were applied: (i) DMSO solvent as control, (ii) HIV Env peptide pool (2 μg/ml), (iii) SIV Nef peptide pool (2 μg/ml), (iv) SIV Gag peptide pool (2 μg/ml), (v) MV proteins, (vi) SEB as positive control (4ug/ml). After washing in staining buffer, cells were stained with a viability dye (violet fluorescent reactive dye, Invitrogen), and then fixed and permeabilized with the BD Cytofix/Cytoperm reagent. Permeabilized cell samples were stored at −80° C. before the staining procedure with the following antibodies: CD3, CD4 and CD8 (used as lineage markers), and INF-g, TNF-α, IL-2 and CD154. After incubation, cells were washed in BD Perm/Wash buffer before to be resuspended in 200 μl of wash buffer and acquired with the BD Canto II Flow Cytometer (BD Biosciences). Flow Cytometry data were analyzed using Flowjo software (TreeStar, OR).

Analysis of antibody responses in serum. The antibody response against SHIV antigens was measured by an enzyme-linked immunosorbent assay (ELISA) using proteins from the NIH AIDS Research and reference Reagent Program (Env protein: gp120 Bal) or the NIBSC (Nef J5 and Gag rp27 proteins) as capture antigens. Anti-MV (Trinity Biotech) antibodies were detected by using commercial ELISA kits. Briefly, 1 μg/mL protein was used to coat a 96-well Nunc Maxisorp microtiter plate. Negative controls consisted of normal cynomolgus macaque serum and saturation assay buffer. The starting dilution of the sera was 1/50, and bound antibodies were detected with goat anti-monkey total Ig conjugated to horseradish peroxidase (Hrp) (Jackson Immunoresearch). Following TMB substrate addition, the optical density of the plates was read at 450 nm. The endpoint ELISA titer of binding antibodies was defined as follow: exp [Ln (dilution>)+(baseline OD)/(OD>−OD<)×Ln (dilution>/dilution<)]. The detection limit of the ELISA was considered to be the starting dilution (1/50) of the test sera.

As described for the plasma antibodies, rectal secretion IgA binding antibodies were sought from fluid collected with Weck-Cel™ sponges using goat anti-monkey IgA (Alpha Diagnostics, San Antonio, TX).

Full hematology. Lymphocyte, eosinophils, and cell blood counts (CBC) were performed using a HMX A/L (Beckman Coulter).

Virus neutralization assays. Neutralization assays were performed as described previously (28). Pseudovirus stocks were collected from the 293T cell supernatants at 48-72 hours after transfection, clarified by centrifugation, divided into small volumes and frozen at −80° C. SHIV SF162p3, HIV1 SF162 and HIV1 QH10, which are infectious virus were propagated in activated human PBMCs. Fivefold serial dilutions of heat-inactivated serum samples were assayed for their inhibitory potential against the Env pseudoviruses using the TZM-bl indicator cell line, with luciferase as the readout as described. TZM-bl cells were plated and cultured overnight in flat-bottomed 96-well plates. A pseudovirus (2000 IU per well) in DMEM with 3.5% (vol/vol) FBS (Hyclone) and 40 μg/ml DEAE-dextran was mixed with serial dilutions of plasma or serum and subsequently added to the plated TZM-bl cells. At 48 hours post-infection, the cells were lysed and luciferase activity was measured using a BioTek Synergy HT multimode microplate reader with Gen 5, v2.0 software. The average background luminescence from a series of uninfected wells was subtracted from each experimental well and infectivity curves were generated using GraphPad Prism v6.0d, where values from experimental wells were compared against a well containing a virus without a test reagent (100% infectivity). Neutralization IC50 titer values were calculated in Graphpad Prism v6.2 (GraphPad, San Diego, CA) using the dose-response inhibition analysis function with variable slope, log-transformed x values and normalized y values.

Statistical analysis. Kaplan-Meier curves and the log-rank Mantel Cox test were used to test for differences of survival curves. Non-parametric Kruskal-Wallis and Dunn's multiple comparisons tests were used to evaluate the immune responses obtained in the three different groups of immunization: MV, Wt and Mt. Wilcoxon matched-pairs signed rank tests were used to compare significance of changes in frequency in comparison with baseline frequencies when performing analysis plasma viremia. The spearman rank correlation method was used for correlations. Statistical analyses were performed using GraphPad Prism v6.2 software (GraphPad, San Diego, CA).

Example 1—Generation of Recombinant MeV Viruses Expressinq SHIV Antiqens

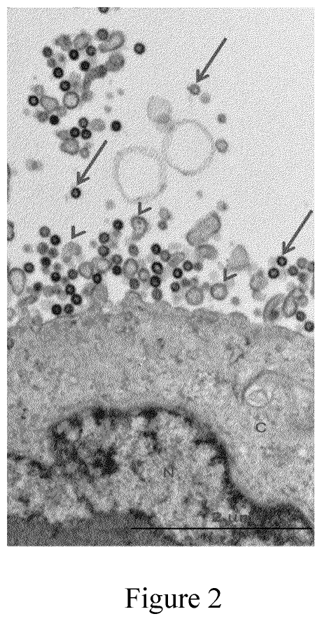

New MV-SHIV vectors expressing simultaneously Gag-Env to form virus-like-particles (VLPs) that we previously demonstrated as very immunogenic (12) were generated (FIG. 2). The sequences corresponding to SIV239 gag and HIV-1 env genes were inserted into two distinct additional transcription units (ATU) (consensus B Env for prime and SF162 Env for boosts) (FIG. 1A). Another MV vector was generated expressing SIV239 Nef under a secreted and non-myristoylated form (FIG. 1B); Nef protein is fused with a signal sequence resulting in its cellular export and lack of myristoylation. Specific vectors were also generated with targeted mutations within the HIV Env and SIV Nef IS domains.

Indeed, HIV possesses not only an IS domain within its Env, likewise other retroviruses, but also within the Nef protein (10-11). Mutations of IS domains have been shown to restore tumor cells sensitivity to immune-rejection (15) and to improve vaccine-immunity (17). Here, both HIV Env and SIV Nef IS domains were mutated, based on the ability of tumor cells expressing the mutated virus proteins to be promptly rejected in vivo compared to tumor cells carrying the wild type forms.

Example 2—Animal Immunization and Vaccine Reqimen

Animals were immunized by subcutaneous route with a prime and two boosts at weeks 13 and 29 (FIG. 3A) with the two MV-SHIV vectors in their wild type or IS domain mutant forms (FIG. 3B). MHC haplotypes associated with natural increased control of HIV/SIV were dispatched in the three groups (25). To evaluate the protective efficacy of the different vaccine regimens, all animals were challenged intra-rectally at week 41 (3 months following the last immunization), once a week with 0.5 AID50 of SHIV-SF162-P3 clade B R5-tropic chimeric virus (a dose that potentially infects 25% of animals at each challenge). Challenges were stopped after two subsequent qRT-PCR virus detections.

Almost all the animals were infected after 5 weeks of challenge (FIG. 4A). However, the viremia was strongly reduced in vaccinated animals as compared to controls, both in yield and in shorter time to clearance, as 75% of vaccinated animals exhibited virus peak values below 10⁴ plasma virus copies/ml (FIG. 4B). Of note, one animal in the mutant group remained uninfected along the study, despite 13 subsequent challenges.

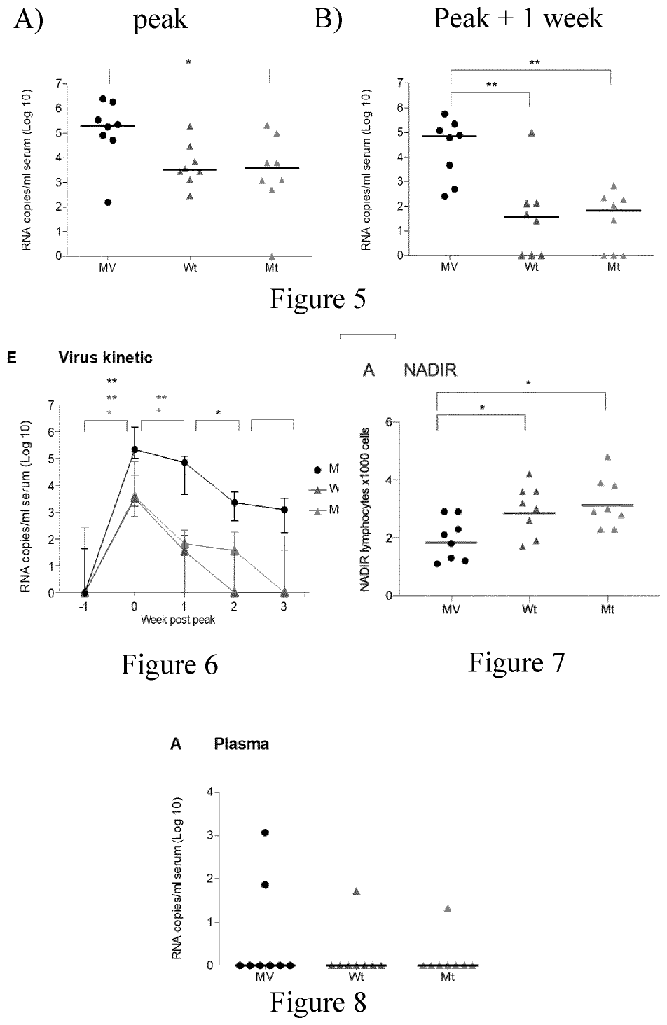

Plasma viremia was strongly reduced in vaccinated animals at peak (mutant group: p<0.05, Kruskal-Wallis and Dunn's multiple comparisons test, FIG. 5A), and one week post-peak with 50 RNA copies/ml or below in 9 out of 16 vaccinated monkeys (p<0.01, FIG. 5B). Vaccination had a strong impact on the kinetic of plasma virus reduction within the first week (p=0.0078 wild-type, p=0.0156 IS domain-mutant, Wilcoxon matched-pairs signed rank tests, FIG. 6). Two weeks post virus peak, more than 50% of vaccinated animals (9 over 16 vaccinated monkeys from the Wt and Mt groups) had no viral RNA in their plasma, thus perfectly controlling the infection, and the others had a strongly reduced viremia as compared to controls (FIG. 6). In contrast, plasma virus RNA copies were still found in 7 out of 8 control monkeys 3 weeks post-peak (FIG. 6, MV group). Vaccination also protected monkeys from lymphocyte depletion (lymphocyte NADIR post-challenge, p<0.05, Kruskal-Wallis and Dunn's multiple comparisons test, FIG. 7).

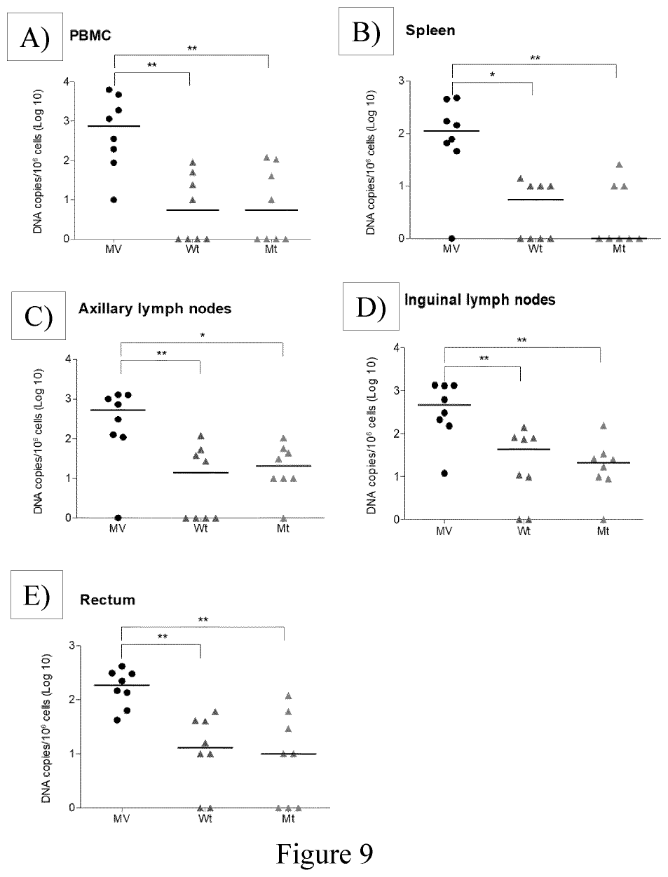

The reduction of plasma virus load in vaccinated animals correlated to the reduction of integrated provirus in PBMCs (p<0.01 Kruskal-Wallis and Dunn's multiple comparisons tests) (FIG. 8 vs. FIG. 9A)), spleen (p<0.05 and p<0.01) (FIG. 8 vs. FIG. 9B), axillary nodes (p<0.05 and p<0.01) (FIG. 8 vs. FIG. 9C), inguinal lymph nodes (p<0.01) (FIG. 8 vs. FIG. 9D) and rectum (p<0.01) (FIG. 8 vs. FIG. 9E). While all control animals were positive for proviral integration in most organs (FIG. 8 and FIG. 9, MV group), a significant proportion of vaccinated animals were negative (8 in PBMC, 9 in Spleen, 5 in axillary lymph nodes, 3 in inguinal lymph nodes and 5 in rectum among the 16 vaccinated animals).

Interestingly, the control of virus reservoirs in long-term non-progressors/elite controller patients has been attributed to superior cytotoxic T lymphocyte (CTL) responses (29).

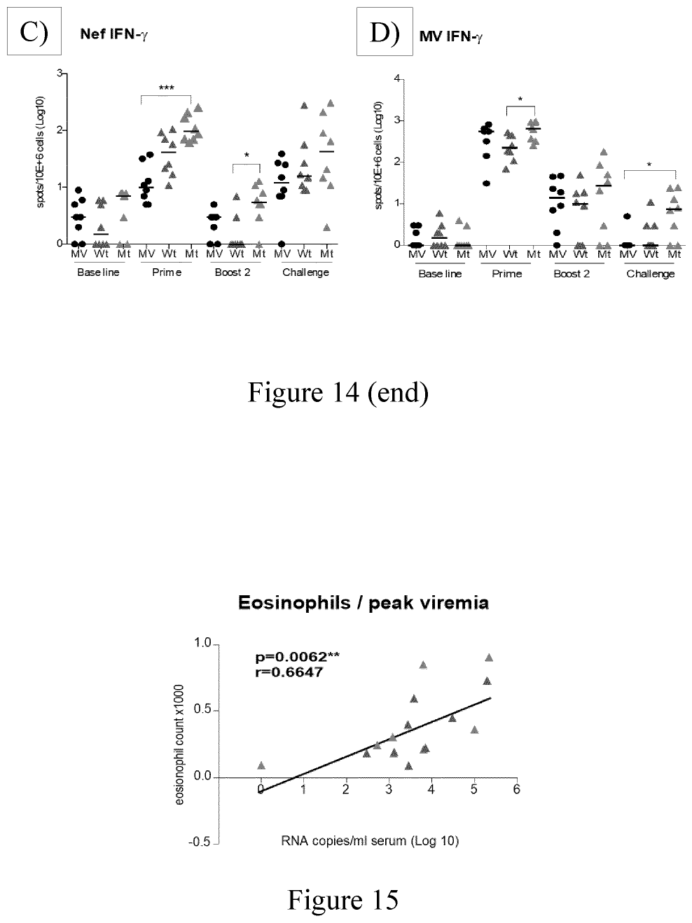

Regarding the role of IS domain mutations in the vaccine composition, we found that anti-Gag and Nef IFNγ cellular immune responses were increased post-prime due to IS domain mutations (p<0.001 compared to controls, Kruskal-Wallis and Dunn's multiple comparisons test) (FIG. 10), in contrast to wild-type antigens (p<0.05 for Gag and non-significant for Nef) (FIG. 10). Although boosting did not improve global cellular immune responses (which is a hallmark of live vaccines that elicit long-term cellular responses after a single immunization) (FIG. 14), higher IL-2 intracellular cytokine levels were measured in animals vaccinated with the IS domain mutants, but not with the IS domain wild type, when compared to control animals (responses specific to Nef p<0.05, Env p<0.05, and Gag p<0.01) (FIG. 11).

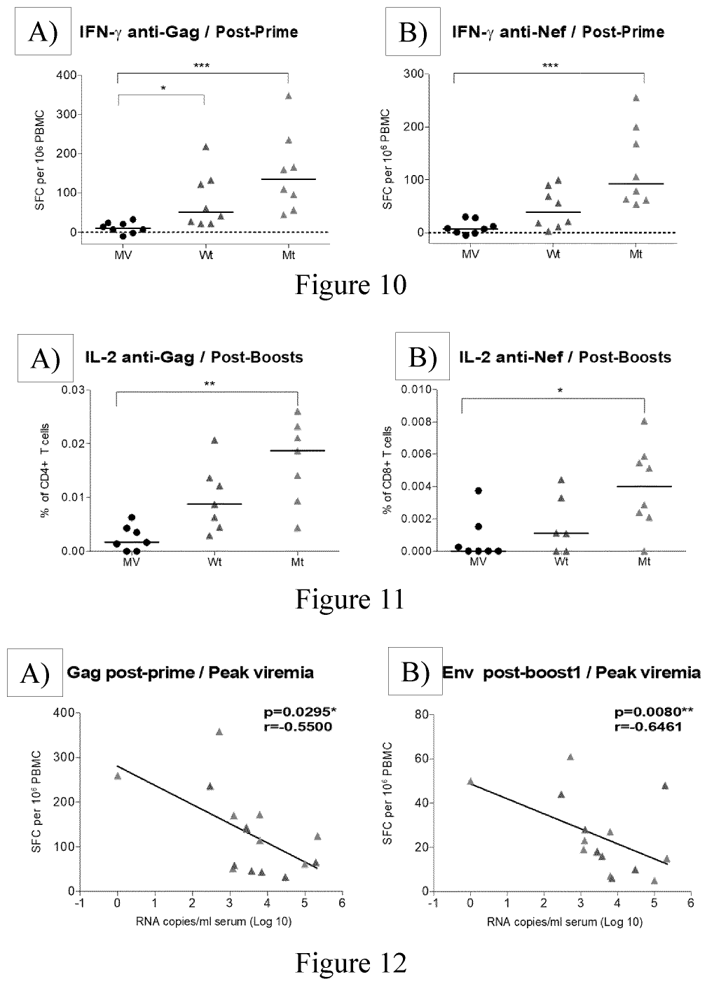

When looking for a correlate of protection, we found that IFNγ cellular immune responses post-prime correlated with reduction of challenge virus peak (anti-Gag, p=0.029, r=−0.5500 non-parametric Spearman correlation, P value: two-tailed), and post-boost (anti-Env, p=0.0080, r=−0,6461) (FIG. 12). The protective role of anti-Gag cellular immune responses was previously reported in monkeys challenged with SHIVSF162p3 (30). In addition, virus-escape mutants have been identified in the STEP trial (Clade B Gag/Pol/Nef) in the vaccinated patients with T-cell epitopes (31), suggesting the strong pressure exerted by the cellular immune system. Efficient cellular immune response with the capacity to differentiate effector cells (TEM) at early replication sites is a hallmark of live vaccines. This was previously shown with a replication-competent cytomegalovirus vaccine expressing SIV proteins that did not prevent initial infection but controlled and eliminated virus in 50% of animals with no detectable antibodies but via a potent stimulation of SIV-specific CTL responses (32, 33).

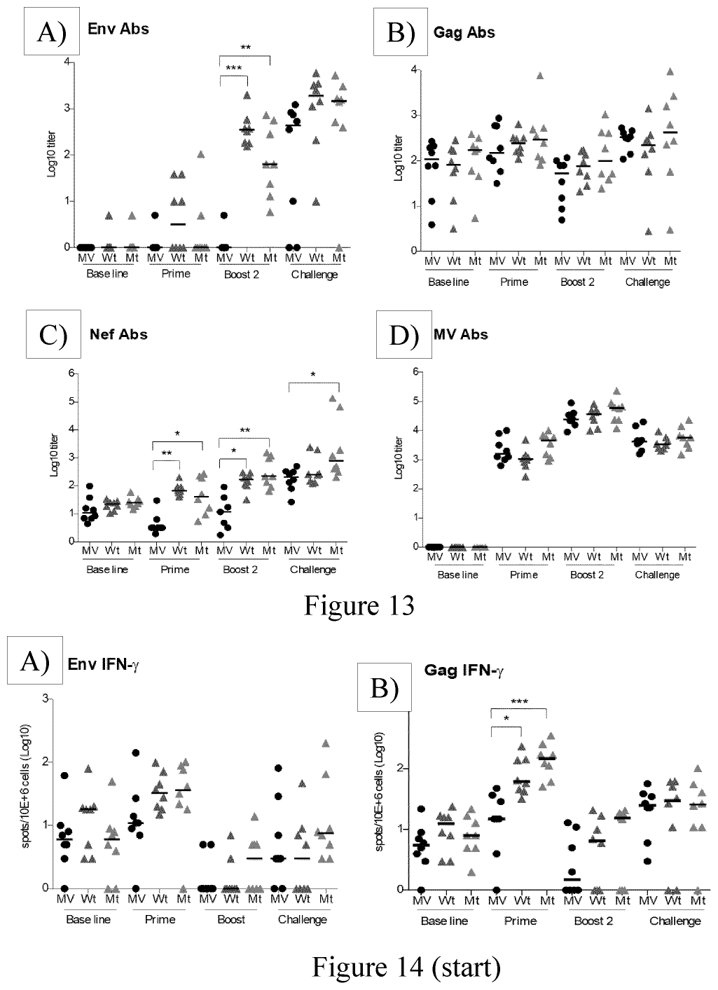

Broadly neutralizing antibodies can be protective in NHP and non-neutralizing antibodies directed to Env variable V2 loop correlated with vaccine-induced protection in the RV144 trial (2, 3). In our study, high levels of antibodies to MV, moderate to HIV Env, and low or even undetectable to SIV Gag and Nef were detected (FIG. 13). Anti-Env antibodies were detected in all animals after the second boost at a relatively high level and were 10-fold increased after challenge in vaccines as anamnestic responses (FIG. 13A). Borderline levels of antibodies to SIV Gag were elicited and were not increased after challenge (FIG. 13B). Antibodies to SIV Nef were induced in all vaccinated animals after boosting and were slightly increased after challenge (p<0.05 for mutant vaccine group compared to controls) (FIG. 13C). No neutralizing antibody was found against the Tier2 SHIVSF162p3 strain, and only a few animals displayed low antibody neutralizing activity after the second boost against the easy to neutralize Tier1 HIV-SF162 strain (Table 1). Although we did not find correlations between antibody titers, especially anti-Env antibodies, and viral loads or SHIV-acquisition, we suppose that vaccine-induced antibodies might contribute to SHIV control as previously shown for non-neutralizing antibodies through antibody dependent cell cytotoxicity (ADCC) and/or antibody dependent cell phagocytosis (ADCP) mechanisms (2, 3).

TABLE 1
Neutralization activity of MV-SHIV-induced antibodies. Assays
of neutralizing activity of serum-antibodies from controls
(measles virus, MV group) or vaccinated macaques with MV-
SHIV wild-type (Wt group) or IS domain mutant (Mt group) against
HIV-1 SF162, SHIV162p3 or HIV-1 QHO pseudo-viruses. Serums
were collected at base line (week −2), boost (4 weeks
post-second boost, week +33), and challenge (3 weeks post
first positive qRT-PCR, W + 3 post infection).

| | | | | HIV-1 | HIV-1 |
|---|---|---|---|---|---|
| | | | SHIV162p3 | SF162 | QH0 |
| | | Animals | IC50 | IC50 | IC50 |
| MV | Base | BV165 | ND | <16 | <16 |
| | line | BW695 | ND | <16 | <16 |
| | | CA117 | ND | <16 | <16 |
| | | CA870N | ND | <16 | <16 |
| | | CBD006 | ND | <16 | <16 |
| | | BV834 | ND | <16 | <16 |
| | | CB135 | ND | <16 | <16 |
| | | CG393 | ND | <16 | <16 |
| | Boost | BV165 | ND | <16 | <16 |
| | | BW695 | ND | <16 | <16 |
| | | CA117 | ND | <16 | <16 |
| | | CA870N | ND | <16 | <16 |
| | | CBD006 | ND | <16 | <16 |
| | | BV834 | ND | <16 | <16 |
| | | CB135 | ND | <16 | <16 |
| | | CG393 | ND | <16 | <16 |
| | Challenge | BV165 | ND | <16 | <16 |
| | | BW695 | ND | <16 | <16 |
| | | CA117 | ND | <16 | <16 |
| | | CA870N | ND | <16 | <16 |
| | | CBD006 | ND | <16 | <16 |
| | | BV834 | ND | <16 | <16 |
| | | CB135 | ND | <16 | <16 |
| | | CG393 | ND | <16 | <16 |
| Wt | Base | BX409 | <16 | <16 | <16 |
| | line | BY250 | <16 | <16 | <16 |
| | | BW430 | <16 | <16 | <16 |
| | | CBE002 | <16 | <16 | <16 |
| | | BZ509 | <16 | <16 | <16 |
| | | CB637 | <16 | <16 | <16 |
| | | BY549 | <16 | <16 | <16 |
| | | BY791 | <16 | <16 | <16 |
| | Boost | BX409 | <16 | 64 | <16 |
| | | BY250 | <16 | <16 | <16 |
| | | BW430 | <16 | <16 | <16 |
| | | CBE002 | <16 | 200 | <16 |
| | | BZ509 | <16 | <16 | <16 |
| | | CB637 | <16 | <16 | <16 |
| | | BY549 | <16 | <16 | <16 |
| | | BY791 | <16 | <16 | <16 |
| | Challenge | BX409 | <16 | 50 | <16 |
| | | BY250 | <16 | 70 | <16 |
| | | BW430 | <16 | 200 | <16 |
| | | CBE002 | <16 | 230 | <16 |
| | | BZ509 | <16 | <16 | <16 |
| | | CB637 | <16 | <16 | <16 |
| | | BY549 | <16 | 150 | <16 |
| | | BY791 | <16 | <16 | <16 |
| Mt | Base | BW821 | <16 | <16 | <16 |
| | line | BX109 | <16 | <16 | <16 |
| | | BX879 | <16 | <16 | <16 |
| | | CG581 | <16 | <16 | <16 |
| | | BV285 | <16 | <16 | <16 |
| | | CBD005 | <16 | <16 | <16 |
| | | CA142 | <16 | <16 | <16 |
| | | CG889 | <16 | <16 | <16 |
| | Boost | BW821 | <16 | <16 | <16 |
| | | BX109 | <16 | <16 | <16 |
| | | BX879 | <16 | <16 | <16 |
| | | CG581 | <16 | <16 | <16 |
| | | BV285 | <16 | <16 | <16 |
| | | CBD005 | <16 | <16 | <16 |
| | | CA142 | <16 | <16 | <16 |
| | | CG889 | <16 | <16 | <16 |
| | Challenge | BW821 | <16 | <16 | <16 |
| | | BX109 | <16 | 250 | <16 |
| | | BX879 | <16 | <16 | <16 |
| | | CG581 | <16 | <16 | <16 |
| | | BV285 | <16 | <16 | <16 |
| | | CBD005 | <16 | <16 | <16 |
| | | CA142 | <16 | 250 | <16 |
| | | CG889 | <16 | <16 | <16 |

IC50: 50% inhibitory concentration,
ND: not done.

We evaluated cell-mediated immune responses by IFNγ ELISPOT assay in response to HIV Env, SIV Gag, SIV Nef, and MV vector antigens (FIG. 14A-D). HIV-Env cellular responses were low and only observed after prime with no increase after challenge (FIG. 14A). In contrast, Gag and Nef cellular immune responses were significantly induced by MV-SHIV prime (FIGS. 14B and C). Boosting did not improve these responses similarly to MV-specific cellular responses (FIG. 14D), which is a feature of live vaccines that induce long-term cellular responses after a single vaccination. Post-prime cellular responses elicited to SHIV and MV antigens waned over time despite two booster immunizations and challenge. We previously made the same observation in macaques and demonstrated that although memory T cell responses to MV vector are hardly detectable in circulating PBMCs and necessitate long in vitro proliferation to be detected, they persist in the secondary lymphoid organs such as lymph nodes and spleen (11).

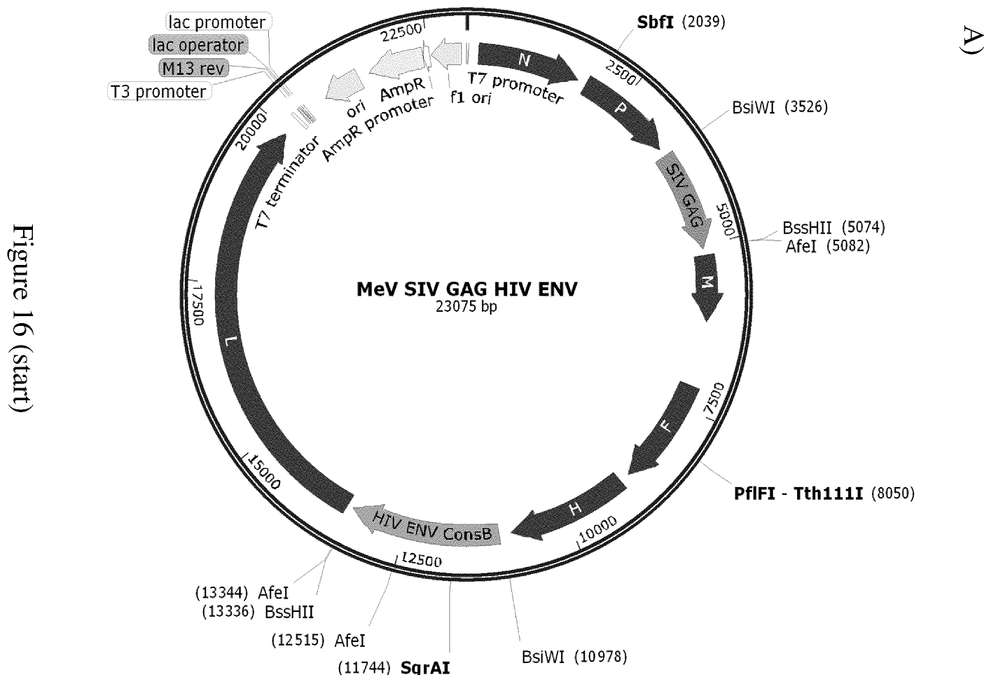

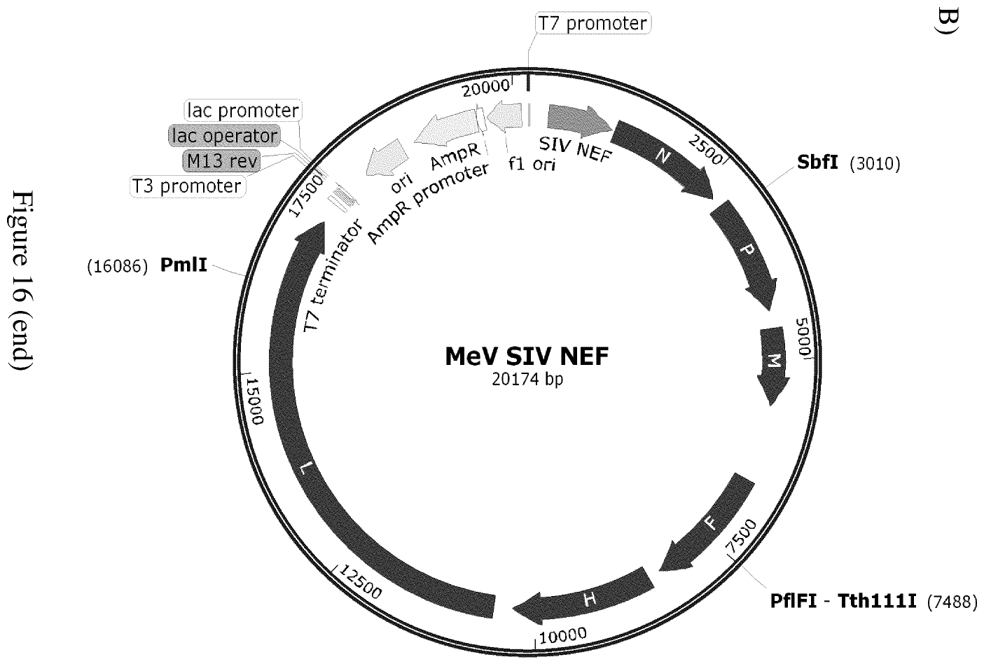

Noteworthy, we observed that the eosinophil cell counts in vaccinated animals positively correlated with the level of viremia (p=0.0207, r=0.5794, Spearman test) (FIG. 15), suggesting that high levels of eosinophil cells could contribute to SHIV infection. Indeed, a prevalence rate of 70% of eosinophilia in SIV-infected macaques compared to 10% in naïve monkeys was previously reported (34), and eosinophilia is described to be associated to HIV infection/AIDS progression (35).

Vaccine protection against neutralization-resistant SHIV162p3 has only been achieved only in a limited number of NHP trials (36-39). CCR5-tropic SHIV162p3 is one of the most stringent NHP challenge model with low level of broadly neutralizing antibody in long-term infected macaques (40). Homologous vaccination with SF162 Env trimer protein did not protect against SHIVSF162p3 acquisition or proviral load in reservoirs (28). Similarly, vaccination with SIVmac251 antigens and challenge with SIVmac251 was not protective in macaques (41). In the perspective of HIV transmission prevention, reducing early plasma virus through an HIV vaccine could play a major role as 50% of human infections occur via donors who are in acute or early stage of infection (42, 43). Moreover, reducing viral load at early stage of infection could delay AIDS outcome, as early initiation of antiretroviral therapy (ART) does (44, 45).

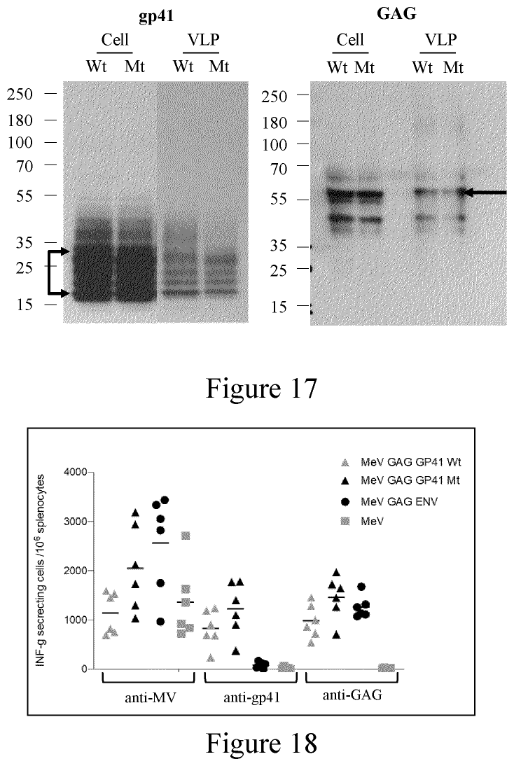

Production of SHIV Antiqens in Cells Infected with a Measles-SHIV Virus Accordinq to the Invention (MeV-SIV-GAG HIV-ENV-Qp41) (FIG. 17) and Immune Response in Mice Vaccinated with the Measles-SHIV Virus of the Invention (FIG. 18).

As illustrated on FIG. 17, MeV-GAG-gp41 leads to the production of GAG and gp41 proteins in infected cells, and also lead to the production of VLPs having GAG and gp41 proteins. As illustrated on FIG. 18, the immune system of mice vaccinated with a MeV-SHIV according to the invention comprising heterologous polynucleotides encoding ENV-gp41 (mutated or not within its IS domain) and GAG responds to the expression and secretion of the encoded antigens; indeed, the cells of the immune systems of vaccinated mice are more responsive to a stimulation with SHIV peptides (they secret more IFNγ than MeV-vaccinated control mice). Further, it can be observed that mice vaccinated with MeV-SHIV virus according to the invention comprising heterologous polynucleotides encoding ENV-gp41 mutated within its IS domain and GAG elicit an improved immune response, as compared to control mice and mice vaccinated with a MeV-SHIV virus encoding wild type gp41 or ENV and GAG.

Elicitation of Immune Response by Prime/Boost Vaccination

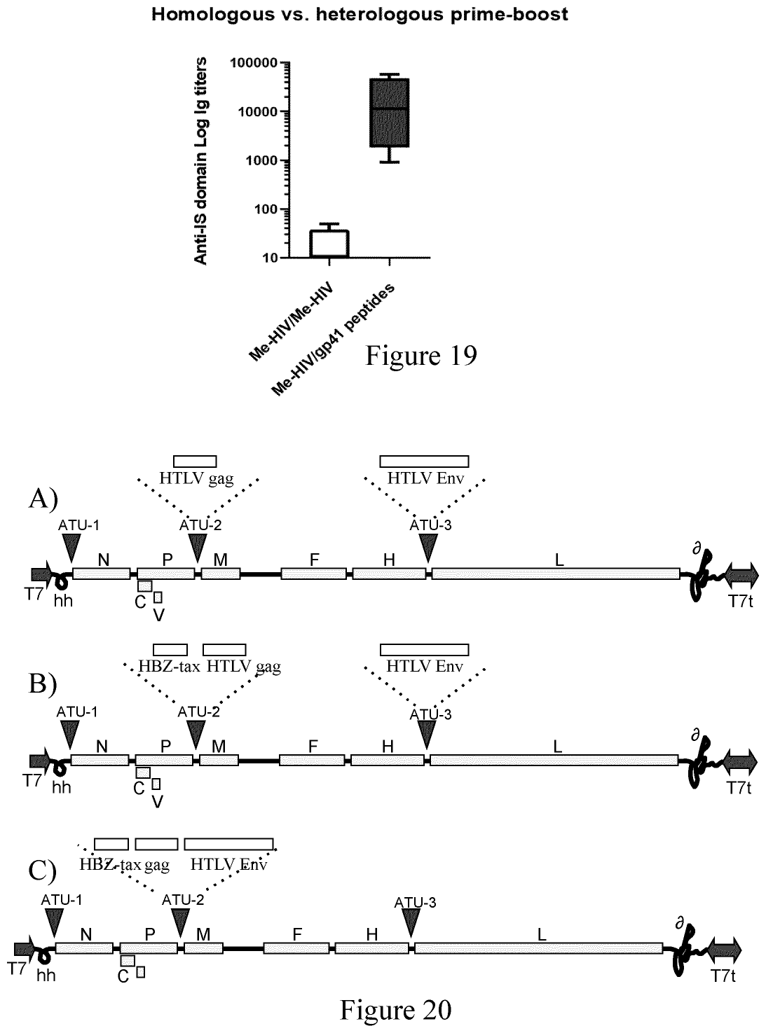

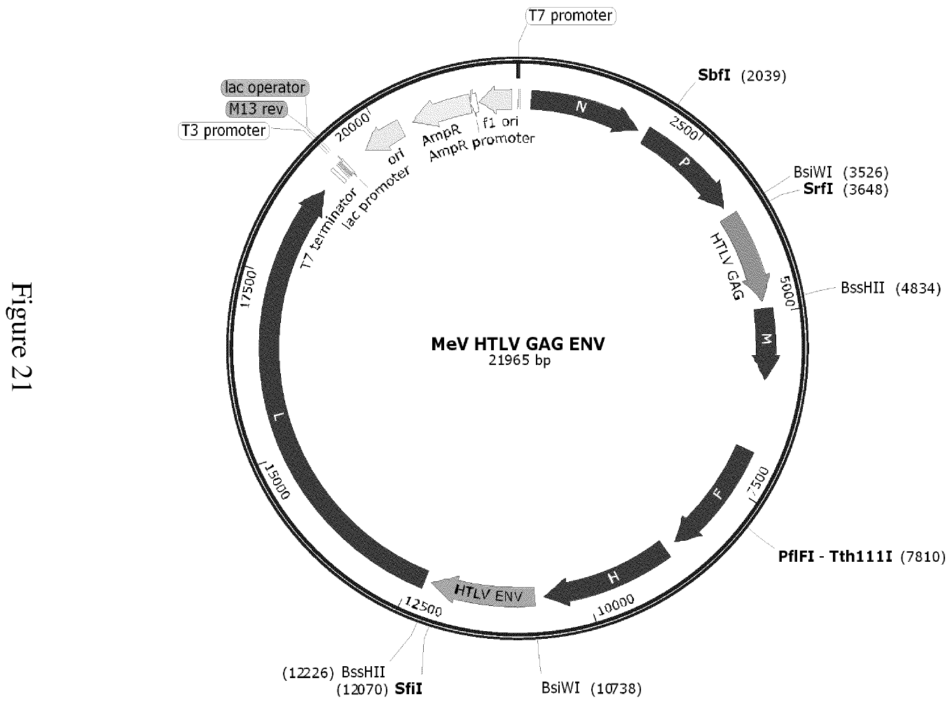

As illustrated on FIG. 19, combining recombinant measles virus (MeV) expressing HIV antigens (MeV-HIV) with heterologous boosts strongly increases immunogenicity, especially humoral responses against specific antigenic domains. These results demonstrate in mice the superiority of heterologous prime/boost vaccination over homologous prime/boost vaccination for the induction of antibodies directed against the immunosuppressive (IS) domain of the HIV envelope. In these experiments, two groups of mice were given at 2 weeks intervals a MeV-HIV/Me-HIV prime/boost (MeV expressing SIV Gag and HIV Env or gp41 antigens) or a MeV-HIV prime followed by a boost consisting of several peptide-adjuvants of the extracellular region of gp41. Measles vectorized HIV vaccination could also be used as a boost following protein (peptide), mRNA-liposome or non-measles viral vector and vaccine antigens.

### Conclusion

This study is the first demonstration that a measles-derived replicating vaccine vector is able to provide some protection from high viremia and reservoir establishment in NHP. Virus control was achieved with no need of heterologous boosting or complex composition and correlated with levels of cellular immune responses. Mutations of IS domains clearly increased vaccine immunogenicity, indicating that they should be included in any other HIV vaccine strategies. The replicative capacity of this human vaccine likely played a major role in providing protection. Measles vaccine platform has already demonstrated clinical feasibility. It is a cheap live vaccine easy to manufacture and to distribute. Preventing AIDS with a pediatric vaccine would be an ideal goal, and clinical studies of the present invention are ongoing to confirm the potential of such MV-HIV-1 vaccine candidate in humans.

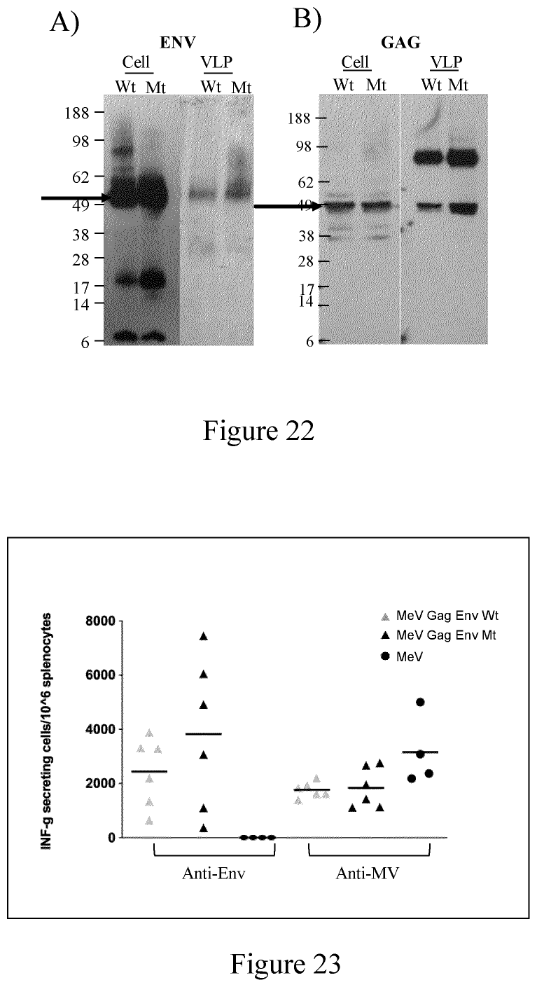

Example 3—Production of HTLV Antiqens in Cells Infected with a Measles-HTLV Virus Accordinq to the Invention (MeV HTLV GAG ENV) and Immune Response in Mice Vaccinated with the Measles-HTLV Virus of the Invention (FIGS. 22 and 23)

HIV and HTLV both belong to the retroviridae family and to the lentivirus and delta-virus genera respectively. These retroviruses have comparable genomic organizations: 3 genes encoding capsid proteins (Gag), attachment proteins (Env) and enzyme proteins (Pro and Pol) framed by transcriptional regulatory domains LTRs (long terminal repeat). HIV and HTLV have different antigenic characteristics and encode distinct proteins which are used in prime/boost vaccine strategies using recombinant measles virus: NEF proteins for HIV and TAX and HBZ for HTLV.

Measles-HIV vaccine induces strong CD8 T cell responses against Gag, Env and Nef antigens (see FIG. 14), which in turn correlate with the reduction in post-challenge viral load found in the non-human primate (FIG. 15). MeV-HIV prime/boost heterologous vaccination strategy applied to HTLV vaccination should lead to high levels of cellular and humoral immune responses in non-human primates. MeV-HTLV vaccination is based on the administration of the vaccine antigens Gag, Env, Tax and HBZ. Yet, vaccination with recombinant vaccinia viruses (rVVs) expressing HTLV-1 basic leucine zipper (bZIP) factor (HBZ) or TAX antigens protected mice inoculated with human HTLV-lymphoma cells and induced strong cellular responses against both HBZ and TAX antigens in non-human primates (Sugata et al., 2015). In addition, several clinical studies have reported the therapeutic benefits of inducing strong T-CD8 cellular responses through autologous transfer of dendritic cells with HTLV TAX CD8 T epitopes in HTLV-infected patients (Suehiro et al., 2015; El Hajj et al., 2020).

Cells infected with a nucleic acid construct of the invention comprising within the cDNA of a MeV a first polynucleotide encoding a GAG antigen from a HTLV (FIG. 22B) and a second polynucleotide encoding a ENV antigen from HTLV (FIG. 22A) produces detectable quantities of GAG and ENV antigen, irrespectively of the nature of ENV (i.e. either wild type version of the antigen or a mutated version thereof). VLPs are also produced by cells infected with the nucleic acid construct, illustrating that a vaccine based on these constructions is likely to induce an immune response within a host. As illustrated on FIG. 23, the immune system of mice vaccinated with a MeV-HTLV according to the invention comprising heterologous polynucleotides encoding ENV (mutated or not within its IS domain) and GAG responds to the expression and secretion of the encoded antigens; indeed, the cells of the immune systems of vaccinated mice are more responsive to a stimulation with HTLV peptides (they secret more IFNγ than control mice, as compared to the negative control). Further, it can be observed that mice vaccinated with MeV-HTLV virus according to the invention comprising heterologous polynucleotides encoding ENV mutated within its IS domain and GAG elicit an improved immune response, as compared to control mice and mice vaccinated with a MeV-HTLV virus encoding wild type ENV and GAG.

### Conclusion

This study is the first demonstration that a measles-derived replicating vaccine vector is able to elicit production of HTLV antigens in host cells. Mutations of the ENV IS domain increases vaccine immunogenicity, indicating that they should be included in vaccine strategies. Measles vaccine platform has already demonstrated clinical feasibility. The replicative capacity of this human vaccine likely played a major role in providing protection. It is a cheap live vaccine easy to manufacture and to distribute. Preventing HTLV with a vaccine would be an ideal goal, and clinical studies of the present invention are ongoing to confirm the potential of such MV-HTLV vaccine candidate in humans.

The invention can be defined according to the following embodiments:

1. A nucleic acid construct which comprises a cDNA molecule encoding a full length antigenomic (+) RNA strand of a measles virus (MeV); and
(i) a first heterologous polynucleotide encoding at least one GAG antigen, a fragment thereof, or a mutated version thereof of a Simian Immunodeficiency Virus (SIV) or a Human Immunodeficiency Virus (HIV), wherein the first heterologous polynucleotide is operatively cloned within an additional transcription unit (ATU) inserted within the cDNA of the antigenomic (+) RNA, in particular an ATU located between the P gene and the M gene of the MeV, in particular in the ATU2 inserted between the P gene and the M gene of the MeV;
(ii) a second heterologous polynucleotide encoding at least one ENV antigen, or a fragment thereof comprising an immunosuppressive domain (ISD), in particular at least one fragment comprising the transmembrane subunit of the ENV antigen, wherein the ENV antigen or its fragment is mutated within its immunosuppressive domain (ISD) and is of a Simian Immunodeficiency Virus (SIV) or a Human Immunodeficiency Virus (HIV), wherein the second heterologous polynucleotide is operatively cloned within the same or a different additional transcription unit (ATU) as in (i) inserted within the cDNA of the antigenomic (+) RNA, in particular an ATU located between the H gene and the L gene of the MeV, in particular in the ATU3 inserted between the H gene and the L gene of the MeV;
wherein the mutation within the ISD domain of ENV reduces the immunosuppressive index of the ENV antigen; and wherein the GAG and ENV antigens, or their respective immunogenic fragments or mutated versions thereof, all originate from the same virus type, in particular are from the same virus strain, more particularly from HIV-1.

2. The nucleic acid construct of embodiment 1, wherein the GAG and ENV antigens are issued from HIV and/or SIV, and which comprises:
(iii) a third heterologous polynucleotide encoding at least one NEF antigen, or a fragment thereof, comprising an immunosuppressive domain (ISD), wherein the NEF antigen is mutated within its ISD domain, and is of a SIV or HIV, wherein the third heterologous polynucleotide is operatively cloned within the same or a different additional transcription unit (ATU) as in (i) or (ii) inserted within the cDNA of the antigenomic (+) RNA, in particular an ATU located upstream the N gene of the MeV, in particular in the ATU1 inserted upstream the N gene of the MeV,
wherein the mutation within the ISD of NEF reduces the immunosuppressive index of the NEF antigen; and wherein the GAG, ENV and NEF antigens, or their respective immunogenic fragments or mutated versions thereof, all originate from the same virus type, in particular are from the same virus strain, more particularly from HIV-1.

3. A combination of nucleic acid constructs which comprises:
(a) the first nucleic acid construct according to embodiment 1 wherein the GAG and ENV antigens are issued from HIV and/or SIV; and
(b) a second nucleic acid construct comprising:
(i′) a second cDNA molecule encoding a full length antigenomic (+) RNA strand of a measles virus (MeV);
(ii′) a third heterologous polynucleotide encoding at least one NEF antigen, or a fragment thereof, mutated within its ISD, of a SIV or HIV, wherein the third heterologous polynucleotide is operatively cloned within an additional transcription unit (ATU) inserted within the cDNA of the antigenomic (+) RNA of (i′), in particular an ATU located upstream the N gene of the MeV, in particular in the ATU1 inserted upstream the N gene of the MeV,
wherein the mutation within the ISD of NEF reduces the immunosuppressive index of the NEF antigen; and wherein the GAG, ENV and NEF antigens or their respective immunogenic fragments or mutated versions thereof, all originate from the same virus type, in particular are from the same virus strain, more particularly from HIV-1.

4. The nucleic acid construct(s) according to any one of embodiments 1 to 3, wherein the first heterologous polynucleotide encodes at least a fragment of an antigen selected from the group consisting of SIV-GAG, SIV-GAGpro, HIV-GAG or HIV-GAGpro, in particular a HIV-1-GAG or HIV-1-GAGpro, in particular an antigen comprising or consisting of the amino acid sequence set forth in SEQ ID No: 1, SEQ ID No: 2, SEQ ID No: 3, SEQ ID No: 4, SEQ ID No: 5 or SEQ ID No: 6.

5. The nucleic acid construct(s) according to any one of embodiments 1 to 4, wherein the second heterologous polynucleotide encodes at least an antigen or a fragment thereof selected from the group consisting of SIV-ENV or HIV-ENV, in particular HIV-1-ENV, in particular an antigen comprising or consisting of the amino acid sequence set forth in SEQ ID No: 8, SEQ ID No: 10, SEQ ID No: 11 or SEQ ID No: 13, or wherein the second heterologous polynucleotide encodes at least a ENV antigen, or a fragment thereof, wherein said antigen or fragment comprises a mutated immunosuppressive domain (ISD), wherein the mutation corresponds to a substitution or a deletion of at least one amino acid residue within its ISD, as compared to a wild type ENV ISD, in particular as compared to the ISD of the ENV polypeptide of SEQ ID No: 7, SEQ ID No: 9 or SEQ ID No: 12.

6. The nucleic acid construct(s) according to embodiment 2, wherein the third heterologous polynucleotide encodes at least a NEF antigen, or a fragment thereof, comprising or consisting of the amino acid sequence of SIV-NEF or HIV-NEF, in particular HIV-1-NEF, in particular an antigen comprising or consisting of the amino acid sequence set forth in SEQ ID No: 15, SEQ ID No: 17 or SEQ ID No: 19, or wherein the third heterologous polynucleotide encodes at least a NEF antigen, or a fragment thereof, said antigen or fragment comprising a mutated immunosuppressive domain (ISD), wherein the mutation corresponds to a substitution or a deletion of at least one amino acid residue within its ISD, as compared to a wild type NEF ISD, in particular as compared to the ISD of the NEF polypeptide of SEQ ID No: 14, SEQ ID No: 16 or SEQ ID No: 18.

7. The nucleic acid construct(s) according to any one of embodiments 2 to 6, wherein the first heterologous antigen encodes at least a fragment of HIV-GAG or HIV-GAGpro, in particular HIV-1-GAG or HIV-1-GAGpro, in particular a HIV-1-GAG comprising or consisting of the amino acid sequence of SEQ ID No: 2 or HIV-1-GAGpro comprising or consisting of amino acid sequence of SEQ ID No: 5,
wherein the second heterologous polynucleotide encodes ENV or a ENV fragment comprising or consisting of the amino acid sequence of HIV consensus B ENV, or the amino acid sequence of SF162 ENV, in particular the amino acid sequence set forth in the group consisting of SEQ ID No: 20 or SEQ ID No: 21, or wherein the second heterologous polynucleotide encodes at least a fragment of an ENV antigen mutated within its immunosuppressive domain (ISD), wherein the mutation corresponds to a substitution or a deletion of at least one amino acid residue within its ISD, as compared to a wild type ENV ISD, in particular as compared to the ISD of the ENV polypeptide of SEQ ID No: 7, SEQ ID No: 9 or SEQ ID No: 12, in particular at least a fragment of an ENV antigen comprising or consisting of the amino acid sequence of SEQ ID No: 8, SEQ ID No: 10, SEQ ID No: 11 or SEQ ID No: 13, and
wherein the third heterologous polynucleotide encodes at least a fragment of a NEF antigen comprising or consisting of the amino acid sequence of SEQ ID No: 15, SEQ ID No: 17 or SEQ ID No: 19, or wherein the third heterologous polynucleotide encodes at least a fragment of a NEF antigen mutated within its immunosuppressive domain (ISD), wherein the mutation corresponds to a substitution or a deletion of at least one amino acid residue within its ISD, as compared to a wild type NEF ISD, in particular as compared to the ISD of the NEF polypeptide of SEQ ID No: 14, SEQ ID No: 16 or SEQ ID No: 18.

8. The nucleic acid construct(s) according to any one of embodiments 1 to 7, wherein the measles virus is an attenuated virus strain selected from the group consisting of the Schwarz strain, the Zagreb strain, the AIK-C strain, the Moraten strain, the Philips strain, the Beckenham 4A strain, the Beckenham 16 strain, the Edmonston seed A strain, the Edmonston seed B strain, the CAM-70 strain, the TD 97 strain, the Leningrad-16 strain, the Shanghai 191 strain and the Belgrade strain, in particular the Schwarz strain.

9. The nucleic acid construct(s) according to any one of embodiments 1 to 8, wherein the first nucleic acid construct has a recombinant cDNA sequence selected from the group consisting of:
SEQ ID No: 32 (construct MeV-SIVgag-HIVenv Cons B WT);
SEQ ID No: 40 (construct MeV-SIVgag-HIVenv Cons B MT);
SEQ ID No: 33 (construct MeV-SIVgag-HIVenv SF162 WT);
SEQ ID No: 41 (construct MeV-SIVgag-HIVenv SF162 MT);
SEQ ID No: 43 (construct MeV-SIVgag-HIVenv gp41 WT); and
SEQ ID No: 44 (construct MeV-SIVgag-HIVenv gp41 MT).

10. An infectious recombinant measles virus, said virus comprising in its genome one nucleic acid construct according to any one of embodiments 1 to 9, in particular wherein the infectious replicating measles virus expresses at least one antigen selected from the group consisting of mutated ENV, GAG, or GAGpro, and optionally mutated NEF antigen, or immunogenic fragments thereof.

11. The infectious replicating recombinant measles virus according to embodiment 10, which elicits a cellular and/or humoral and cellular response, in particular after a prime-boost immunization, more particularly after a homologous prime-boost immunization, against the immunogenic antigen(s) of the GAG, ENV and/or NEF antigens if any, or immunogenic fragments thereof, in particular a T cell response, in particular a IFNγ and/or a IL-2 response.

12. A host cell transfected with the combination of nucleic acid constructs according to any one of embodiments 1 to 9, or infected with the recombinant measles virus according to embodiment 10 or 11, in particular a mammalian cell, a VERO NK cells, CEF cells, or human embryonic kidney cell line 293T.

13. Recombinant virus like particles (VLPs) comprising a GAG and a ENV antigen, and optionally a NEF antigen, or immunogenic fragments thereof, of SIV and/or HIV, wherein the antigen or immunogenic fragments thereof are encoded by the first, the second, and optionally the third, heterologous polynucleotides of the nucleic acid constructs according to embodiments 1 to 9, or the recombinant measles virus according to embodiment 10 or 11, or produced within the host cell of embodiment 12.

14. An immunogenic composition, especially a virus vaccine composition, comprising the infectious replicating recombinant measles virus according to embodiment 10 or 11, the recombinant VLPs according to embodiment 13, or the recombinant measles virus according to embodiment 10 or 11 and the recombinant VLPs according to embodiment 13, and a pharmaceutically acceptable vehicle.

15. The composition according to embodiment 14 for use in the elicitation of a protective, and preferentially prophylactic, immune response against HIV or SIV by the elicitation of antibodies directed against HIV and/or SIV polypeptides or antigenic fragments thereof or mutated version thereof, and/or a cellular or humoral and cellular response against the HIV and/or SIV, in a host in need thereof, in particular a human host, in particular a child.

16. The composition of embodiment 14 or 15 for use in the elicitation of a protective, and preferentially prophylactic, immune response against measles virus by the elicitation of antibodies directed against measles virus protein(s), and/or a cellular and/or humoral and cellular response against the measles virus, in a host in need thereof, in particular a human host, in particular a child.

17. A method for preventing or treating a HIV or SIV related disease, said method comprising the immunization of a mammalian, especially a human, in particular a child, by the injection, in particular mucosal or intramuscular or subcutaneous injection, more particularly mucosal injection, and most particularly nasal injection, of recombinant Virus Like Particles according to embodiment 13, and/or a measles virus according to embodiment 10 or 11.

The invention can be defined according to the following embodiments:

1. A nucleic acid construct which comprises a cDNA molecule encoding a full length antigenomic (+) RNA strand of a measles virus (MeV); and
(i) a first heterologous polynucleotide encoding at least one GAG antigen, a fragment thereof, or a mutated version thereof of a Human T-lymphotropic virus (HTLV), wherein the first heterologous polynucleotide is operatively cloned within an additional transcription unit (ATU) inserted within the cDNA of the antigenomic (+) RNA, in particular an ATU located between the P gene and the M gene of the MeV, in particular in the ATU2 inserted between the P gene and the M gene of the MeV;
(ii) a second heterologous polynucleotide encoding at least one ENV antigen, or a fragment thereof comprising an immunosuppressive domain (ISD), in particular at least one fragment comprising the transmembrane subunit of the ENV antigen, wherein the ENV antigen or its fragment is mutated within its immunosuppressive domain (ISD) and is of a Human T-lymphotropic virus (HTLV), wherein the second heterologous polynucleotide is operatively cloned within the same or a different additional transcription unit (ATU) as in (i) inserted within the cDNA of the antigenomic (+) RNA, in particular an ATU located between the H gene and the L gene of the MeV, in particular in the ATU3 inserted between the H gene and the L gene of the MeV;
wherein the mutation within the ISD domain of ENV reduces the immunosuppressive index of the ENV antigen; and wherein the GAG and ENV antigens, or their respective immunogenic fragments or mutated versions thereof, all originate from the same virus type, in particular are from the same virus strain, more particularly from HTLV-1 or HTLV-2 or HTLV-3.

2. The nucleic acid construct of embodiment 1, which comprises:
(iii) a third heterologous polynucleotide encoding at least one HBZ antigen, or a fragment thereof, or a mutated version thereof, and is of HTLV, wherein the third heterologous polynucleotide is operatively cloned within the same or a different additional transcription unit (ATU) as in (i) or (ii) inserted within the cDNA of the antigenomic (+) RNA, in particular an ATU located upstream the N gene of the MeV, in particular in the ATU1 inserted upstream the N gene of the MeV, wherein the GAG, ENV and HBZ antigens, or their respective immunogenic fragments or mutated versions thereof, all originate from the same virus type, in particular are from the same virus strain, more particularly from HTLV-1 or HTLV-2 or HTLV-3.

3. The nucleic acid construct(s) according to embodiment 1 or 2, wherein the first heterologous polynucleotide encodes at least a fragment of an antigen selected from the group consisting of HTLV-GAG or HTLV-GAGpro, in particular HTLV-1-GAG or HTLV-1-GAGpro, in particular an antigen comprising or consisting of the amino acid sequence set forth in SEQ ID No: 1 or SEQ ID No: 2.

4. The nucleic acid construct(s) according to any one of embodiments 1 to 3, wherein the second heterologous polynucleotide encodes at least an antigen or a fragment thereof of HTLV ENV, in particular HTLV-1-ENV or HTLV-2-ENV, in particular an antigen comprising or consisting of the amino acid sequence set forth in SEQ ID No: 4 or SEQ ID No: 11, or wherein the second heterologous polynucleotide encodes at least a ENV antigen, or a fragment thereof, wherein said antigen or fragment comprises a mutated immunosuppressive domain (ISD), wherein the mutation corresponds to a substitution or a deletion of at least one amino acid residue within its ISD, as compared to a wild type HTLV ENV ISD, in particular as compared to the ISD of the HTLV ENV polypeptide of SEQ ID No: 3 or SEQ ID No: 10.

5. The nucleic acid construct(s) according to any one of embodiments 2 to 4, wherein the first heterologous antigen encodes at least a fragment of HTLV-GAG, in particular HTLV-1-GAG, comprising or consisting of the amino acid sequence of SEQ ID No: 1 or HTLV-1-GAGpro comprising or consisting of the amino acid sequence of SEQ ID No: 2,
wherein the second heterologous polynucleotide encodes ENV or a ENV fragment comprising or consisting of the amino acid sequence of HTLV ENV, in particular the amino acid sequence set forth in SEQ ID No: 4 or SEQ ID No: 11, or wherein the second heterologous polynucleotide encodes at least a fragment of an ENV antigen mutated within its immunosuppressive domain (ISD), wherein the mutation corresponds to a substitution or a deletion of at least one amino acid residue within its ISD, as compared to a wild type ENV ISD, in particular as compared to the ISD of the ENV polypeptide of SEQ ID No: 3 or SEQ ID No: 10, in particular at least a fragment of an ENV antigen comprising or consisting of the amino acid sequence of SEQ ID No: 4 or SEQ ID No: 11, and
wherein the third heterologous polynucleotide encodes at least a fragment of a HBZ antigen of HTLV comprising or consisting of the amino acid sequence of SEQ ID No: 5 or SEQ ID No: 9, or wherein the third heterologous polynucleotide encodes at least a fragment of a HBZ antigen, wherein HBZ is mutated to reduce its oncogenic properties, in particular wherein the mutation corresponds to a substitution or a deletion of at least one amino acid residue within HBZ, as compared to a wild type HBZ of SEQ ID No: 19, and which is in particular a HBZ antigen associated with at least a fragment of a TAX antigen of the amino acid residue of SEQ ID No: 9.

6. The nucleic acid construct(s) according to any one of embodiments 1 to 5, wherein the measles virus is an attenuated virus strain selected from the group consisting of the Schwarz strain, the Zagreb strain, the AIK-C strain, the Moraten strain, the Philips strain, the Beckenham 4A strain, the Beckenham 16 strain, the Edmonston seed A strain, the Edmonston seed B strain, the CAM-70 strain, the TD 97 strain, the Leningrad-16 strain, the Shanghai 191 strain and the Belgrade strain, in particular the Schwarz strain.

7. The nucleic acid construct(s) according to any one of embodiments 1 to 6, wherein the first nucleic acid construct has the recombinant cDNA sequence of SEQ ID No: 12 (construct MeV-HTLVgag-HTLVenv).

8. An infectious recombinant measles virus, said virus comprising in its genome one nucleic acid construct according to any one of embodiments 1 to 7, in particular wherein the infectious replicating measles virus expresses at least one antigen selected from the group consisting of mutated ENV, GAG, or GAGpro, and optionally mutated HBZ antigen, or immunogenic fragments thereof.

9. The infectious replicating recombinant measles virus according to embodiments 8, which elicits a cellular and/or humoral and cellular response, in particular after a prime-boost immunization, more particularly after a homologous prime-boost immunization, against the immunogenic antigen(s) of the GAG, ENV and/or HBZ antigens if any, or immunogenic fragments thereof, in particular a T cell response, in particular a IFNγ and/or a IL-2 response.

10. A host cell transfected with the combination of nucleic acid constructs according to any one of embodiments 1 to 7, or infected with the recombinant measles virus according to embodiment 8 or 9, in particular a mammalian cell, a VERO NK cells, CEF cells, or human embryonic kidney cell line 293T.

11. Recombinant virus like particles (VLPs) comprising a GAG and a ENV antigen, and optionally a HBZ antigen, or immunogenic fragments thereof, of HTLV, wherein the antigen or immunogenic fragments thereof are encoded by the first, the second, and optionally the third, heterologous polynucleotides of the nucleic acid constructs according to embodiments 1 to 7, or the recombinant measles virus according to embodiment 8 or 9, or produced within the host cell of embodiment 10.

12. An immunogenic composition, especially a virus vaccine composition, comprising the infectious replicating recombinant measles virus according to embodiment 8 or 9, the recombinant VLPs according to embodiment 11, or the recombinant measles virus according to embodiment 8 or 9 and the recombinant VLPs according to embodiment 11, and a pharmaceutically acceptable vehicle.

13. The composition according to embodiment 12 for use in the elicitation of a protective, and preferentially prophylactic, immune response against HTLV by the elicitation of antibodies directed against HTLV polypeptides or antigenic fragments thereof or mutated version thereof, and/or a cellular or humoral and cellular response against the HTLV, in a host in need thereof, in particular a human host, in particular a child.

14. The composition of embodiment 12 or 13 for use in the elicitation of a protective, and preferentially prophylactic, immune response against measles virus by the elicitation of antibodies directed against measles virus protein(s), and/or a cellular and/or humoral and cellular response against the measles virus, in a host in need thereof, in particular a human host, in particular a child.

15. A method for preventing or treating a HTLV related disease, said method comprising the immunization of a mammalian, especially a human, in particular a child, by the injection, in particular mucosal or intramuscular or subcutaneous injection, more particularly mucosal injection, and most particularly nasal injection, of recombinant Virus Like Particles according to embodiment 11, and/or a measles virus according to embodiment 8 or 9.

### BIBLIOGRAPHIC REFERENCES

1. Ng'uni T, Chasara C, Ndhlovu Z M, Major Scientific Hurdles in HIV Vaccine Development: Historical Perspective and Future Directions. Front Immunol. 11, 590780 (2020).
2. S. Rerks-Ngarm, P. Pitisuttithum, S. Nitayaphan, J. Kaewkungwal, J. Chiu, R. Paris, N. Premsri, C. Namwat, M. de Souza, E. Adams, M. Benenson, S. Gurunathan, J. Tartaglia, J. G. McNeil, D. P. Francis, D. Stablein, D. L. Birx, S. Chunsuttiwat, C. Khamboonruang, P. Thongcharoen, M. L. Robb, N. L. Michael, P. Kunasol, J. H. Kim, MOPH-TAVEG Investigators, Vaccination with ALVAC and AIDSVAX to prevent HIV-1 infection in Thailand. N. Engl. J. Med. 361, 2209-2220 (2009).
3. B. F. Haynes, P. B. Gilbert, M. J. McElrath, S. Zolla-Pazner, G. D. Tomaras, S. M. Alam, D. T. Evans, D. C. Montefiori, C. Karnasuta, R. Sutthent, H. X. Liao, A. L. DeVico, G. K. Lewis, C. Williams, A. Pinter, Y. Fong, H. Janes, A. DeCamp, Huang Y, M. Rao, E. Billings, N. Karasavvas, M. L. Robb, V. Ngauy, M. S. de Souza, R. Paris, G. Ferrari, R. T. Bailer, K. A. Soderberg, C. Andrews, P. W. Berman, N. Frahm, S. C. De Rosa, M. D. Alpert, N. L. Yates, X. Shen, R. A. Koup, P. Pitisuttithum, J. Kaewkungwal, S. Nitayaphan, S. Rerks-Ngarm, N. L. Michael, J. H. Kim, Immune-correlates analysis of an HIV-1 vaccine efficacy trial. N. Engl. J. Med. 366(14), 1275-86 (2012).
4. S. P. Buchbinder, D. V. A Mehrotra, Duerr, D. W. Fitzgerald, R. Mogg, D. Li, P. B. Gilbert, J. R. Lama, M. Marmor, C. Del Rio, M. J. McElrath, D. R. Casimiro, K. M. Gottesdiener, J. A. Chodakewitz, L. Corey, M. N. Robertson; Step Study Protocol Team, Efficacy assessment of a cell-mediated immunity HIV-1 vaccine (the Step Study): a double-blind, randomised, placebo-controlled, test-of-concept trial. Lancet 372(9653), 1881-1893 (2008).
5. P. Pitisuttithum, P. Gilbert, M. Gurwith, W. Heyward, M. Martin, F. van Griensven, D. Hu, J. W. Tappero, K. Choopanya, Bangkok Vaccine Evaluation Group, Randomized, double-blind, placebo-controlled efficacy trial of a bivalent recombinant glycoprotein 120 HIV-1 vaccine among injection drug users in Bangkok, Thailand. J. Infect. Dis. 194(12), 1661-71 (2006).
6. N. M. Flynn, D. N. Forthal, C. D. Harro, F. N. Judson, K. H. Mayer, M. F. Para; rgp120 HIV Vaccine Study Group, Placebo-controlled phase 3 trial of a recombinant glycoprotein 120 vaccine to prevent HIV-1 infection. J. Infect. Dis. 191(5), 654-65 (2005).
7. P. N. Frantz, S. Teeravechyan, F. Tangy, Measles-derived vaccines to prevent emerging viral diseases. Microbes Infect. pii: S1286-4579(18), 30038-8 (2018).
8. K. Ramsauer, M. Schwameis, C. Firbas, M. Müllner, R. J. Putnak, S. J. Thomas, P. Desprès, E. Tauber, B. Jilma, F. Tangy, Immunogenicity, safety, and tolerability of a recombinant measles-virus-based chikungunya vaccine: a randomised, double-blind, placebo-controlled, active-comparator, first-in-man trial. Lancet Infect. Dis. 15(5), 519-27 (2015).
9. J. F. Enders, and T. C. Peebles, Propagation in tissue cultures of cytopathogenic agents from patients with measles. Proc. Soc. Exp. Biol. Med. 86, 277-286 (1954)
10. B. N. Fields D. M. Knipe, P. M. Howley, Fields virology 3^{rd} edition, vol. 1, Lippincott-Raven publishers (1996).
11. R. Stebbings, M. Février, B. Li, C. Lorin, M. Koutsoukos, E. T. Mee, N. Rose, J. Hall, M. Page, N. Almond, G. Voss, F Tangy, Immunogenicity of a recombinant measles-HIV-1 clade B candidate vaccine, PLoS One 7(11), e50397 (2012).
12. M. Guerbois, A. Moris, C. Combredet, V. Najburg, C. Ruffié, M. Février, N. Cayet, S. Brandler, O. Schwartz, F. Tang, Live attenuated measles vaccine expressing HIV-1 Gag virus like particles covered with gp160DeltaV1V2 is strongly immunogenic. Virology 388(1), 191-203 (2009).
13. M. Renard, M. Mangeney, T. Heidmann, Mutated HIV Nef for modulating immunity. patent number WO 2006/018289 (2006).
14. T. Heidmann, Mutated lentiviral Env proteins and their use as drugs. patent number WO 2013/083799 (2013).
15. M. Mangeney, M. Renard, G. Schlecht-Louf, I. Bouallaga, O. Heidmann, C. Letzelter, A. Richaud, B. Ducos, T. Heidmann, Placental syncytins: Genetic disjunction between the fusogenic and immunosuppressive activity of retroviral envelope proteins. Proc. Natl. Acad. Sci. U.S.A. 104, 20534-20539 (2007).
16. G. Schlecht-Louf, M. Renard, M. Mangeney, C. Letzelter, A. Richaud, B. Ducos, I. Bouallaga, T. Heidmann, Retroviral infection in vivo requires an immune escape virulence factor encrypted in the envelope protein of oncoretroviruses, Proc. Natl. Acad. Sci. U.S.A. 107(8), 3782-7 (2010).
17. G. Schlecht-Louf, M. Mangeney, H. El-Garch, V. Lacombe, H. Poulet, T. Heidmann, A targeted mutation within the feline leukemia virus (FeLV) envelope protein immunosuppressive domain to improve a canarypox virus-vectored FeLV vaccine. J. Virol. 88(2), 992-1001 (2014).
18. M. Mangeney, T. Heidmann. Tumor cells expressing a retroviral envelope escape immune rejection in vivo. Proc. Natl. Acad. Sci. USA. 95(25), 14920-5 (1998).
19. M. Mangeney, N. de Parseval, G. Thomas, T. Heidmann. The full-length envelope of an HERV-H human endogenous retrovirus has immunosuppressive properties. J Gen Virol. 82(Pt 10), 2515-18 (2001).
20. S. M. Horikami, S. A. Moyer, Structure, transcription, and replication of measles virus. Curr Top Microbiol Immunol. 191, 35-50 (1995).
21. C. Combredet, V. Labrousse, L. Mollet, C. Lorin, F. Delebecque, B. Hurtrel, H. McClure, M. B. Feinberg, M. Brahic, F. Tangy, A molecularly cloned Schwarz strain of measles virus vaccine induces strong immune responses in macaques and transgenic mice. Journal of virology 77(21), 11546-54 (2003).
22. G. Neumann, M. A. Whitt, Y. Kawaoka, A decade after the generation of a negative-sense RNA virus from cloned cDNA—what have we learned? J Gen Virol. 83(Pt 11), 2635-62 (2002).
23. L. Bénit, P. Dessen, T. Heidmann, Identification, phylogeny, and evolution of retroviral elements based on their envelope genes. Journal of virology 75(23), 11709-19 (2001)
24. R Cattaneo, K. Kaelin, K. Baczko, M. A. Billeter, Measles virus editing provides an additional cysteine-rich protein. Cell 56(5), 759-4 (1989).
25. E. T. Mee, N. Berry, C. Ham, U. Sauermann, M. T. Maggiorella, F. Martinon, E. J. Verschoor, J. L. Heeney, R. Le Grand, F. Titti, N. Almond, N. J. Rose, Mhc haplotype H6 is associated with sustained control of SIVmac251 infection in Mauritian cynomolgus macaques. Immunogenetics 61(5), 327-39 (2009).
26. I. Karlsson, B. Malleret, P. Brochard, B. Delache, J. Calvo, R. Le Grand, B. Vaslin, FoxP3+ CD25+ CD8+ T-cell induction during primary simian immunodeficiency virus infection in cynomolgus macaques correlates with low CD4+ T-cell activation and high viral load. J. Virol. 81, 13444-13455 (2007).
27. S. Bernard-Stoecklin, C. Gommet, A. B. Corneau, S. Guenounou, C. Torres, N. Dejucq-Rainsford, A. Cosma, N. Dereuddre-Bosquet, R. Le Grand, Semen CD4+ T cells and macrophages are productively infected at all stages of SIV infection in macaques. PLoS Pathog. 9, e1003810 (2013).
28 R. Le Grand, N. Dereuddre-Bosquet, S. Dispinseri, L. Gosse, D. Desjardins, X. Shen, M. Tolazzi, C. Ochsenbauer, H. Saidi, G. Tomaras, M. Prague, S. W. Barnett, R. Thiebaut, A. Cope, G. Scarlatti, R. J. Shattock, Superior Efficacy of a Human Immunodeficiency Virus Vaccine Combined with Antiretroviral Prevention in Simian-Human Immunodeficiency Virus-Challenged Nonhuman Primates. J. Virol. 90(11), 5315-5328 (2016).
29. S. A. Migueles, D. Mendoza, M. G. Zimmerman, K. M. Martins, S. A. Toulmin, E. P. Kelly, B. A. Peterson, S. A. Johnson, E. Galson, K. O. Poropatich, A. Patamawenu, H. Imamichi, A. Ober, C. A. Rehm, S. Jones, C. W. Hallahan, D. A. Follmann, M. Connors, CD8(+) T-cell Cytotoxic Capacity Associated with Human Immunodeficiency Virus-1 Control Can Be Mediated through Various Epitopes and Human Leukocyte Antigen Types. EBioMedicine 2(1), 46-58 (2014).
30. N. Trunova, L. Tsai, S. Tung, E. Schneider, J. Harouse, A. Gettie, V. Simon, J. Blanchard, C. Cheng-Mayer, Progestin-based contraceptive suppresses cellular immune responses in SHIV-infected rhesus macaques. Virology 352(1), 169-77 (2006).
31. M. Rolland, S. Tovanabutra, A. C. deCamp, N. Frahm, P. B. Gilbert, E. Sanders-Buell, L. Heath, C. A. Magaret, M. Bose, A. Bradfield, A. O'Sullivan, J. Crossler, T. Jones, M. Nau, K. Wong, H. Zhao, D. N. Raugi, S. Sorensen, J. N. Stoddard, B. S. Maust, W. Deng, J. Hural, S. Dubey, N. L. Michael, J. Shiver, L. Corey, F. Li, S. G. Self, J. Kim, S. Buchbinder, D. R. Casimiro, M. N. Robertson, A. Duerr, M. J. McElrath, F. E. McCutchan, J. I. Mullins, Genetic impact of vaccination on breakthrough HIV-1 sequences from the STEP trial. Nat. Med. 17(3), 366-71 (2011).
32. S. G. Hansen, C. Vieville, N. Whizin, L. Coyne-Johnson, D. C. Siess, D. D. Drummond, A. W. Legasse, M. K. Axthelm, K. Oswald, C. M. Trubey, M. Jr. Piatak, J. D. Lifson, J. A. Nelson, M. A. Jarvis, L. J. Picker, Effector memory T cell responses are associated with protection of rhesus monkeys from mucosal simian immunodeficiency virus challenge. Nat Med. 15(3), 293-9 (2009).
33. S. G. Hansen, J. C. Ford, M. S. Lewis, A. B. Ventura, C. M. Hughes, L. Coyne-Johnson, N. Whizin, K. Oswald, R. Shoemaker, T. Swanson, A. W. Legasse, M. J. Chiuchiolo, C. L. Parks, M. K. Axthelm, J. A. Nelson, M. A. Jarvis, M. Jr. Piatak, J. D. Lifson, L. J. Picker, Profound early control of highly pathogenic SIV by an effector memory T-cell vaccine. Nature 473(7348), 523-7 (2011).
34. A. F. Gill, M. H. Ahsan, A. A. Lackner, R. S. Veazey, Hematologic abnormalities associated with simian immunodeficieny virus (SIV) infection mimic those in HIV infection. J. Med. Primatol. 41(3), 214-24 (2012).
35. M. Sivaram, A. White, K. W. Radcliffe, Eosinophilia: clinical significance in HIV-infected individuals. Int. J. STD. AIDS. 23(9), 635-8 (2012).
36. M. Bomsel, D, Tudor, A. S. Drillet, A. Alfsen, Y. Ganor, M. G. Roger, N. Mouz, M. Amacker, A. Chalifour, L. Diomede, G. Devillier, Z. Cong, Q. Wei, H. Gao, C. Qin, G. B. Yang, R. Zurbriggen, L. Lopalco, S. Fleury, Immunization with HIV-1 gp41 subunit virosomes induces mucosal antibodies protecting nonhuman primates against vaginal SHIV challenges. Immunity 34(2), 269-80 (2011).
37. D. H. Barouch, K. E. Stephenson, E. N. Borducchi, K. Smith, K. Stanley, A. G. McNally, J. Liu, P. Abbink, L. F. Maxfield, M. S. Seaman, A. S. Dugast, G. Alter, M. Ferguson, W. Li, P. L. Earl, B. Moss, E. E. Giorgi, J. J. Szinger, L. A. Eller, E. A. Billings, M. Rao, S. Tovanabutra, E. Sanders-Buell, M. Weijtens, M. G. Pau, H. Schuitemaker, M. L. Robb, J. H. Kim, B. T. Korber, N. L. Michael, Protective efficacy of a global HIV-1 mosaic vaccine against heterologous SHIV challenges in rhesus monkeys. Cell 155(3), 531-9 (2013).
38. D. H. Barouch, F. L. Tomaka, F. Wegmann, D. J. Stieh, G. Alter, M. L. Robb, N. L. Michael, L. Peter, J. P. Nkolola, E. N. Borducchi, A. Chandrashekar, D. Jetton, K. E. Stephenson, W. Li, B. Korber, G. D. Tomaras, D. C. Montefiori, G. Gray, N. Frahm, M. J McElrath, L. Baden, J. Johnson, J. Hutter, E. Swann, E. Karita, H. Kibuuka, J. Mpendo, N. Garrett, K. Mngadi, K. Chinyenze, F. Priddy, E. Lazarus, F. Laher, S. Nitayapan, P. Pitisuttithum, S. Bart, T. Campbell, R. Feldman, G. Lucksinger, C. Borremans, K. Callewaert, R. Roten, J. Sado, L. Scheppler, M. Weijtens, K. Feddes-de Boer, D. van Manen, J. Vreugdenhil, R. Zahn, L. Lavreys, S. Nijs, J. Tolboom, J. Hendriks, Z. Euler, M. G Pau, H. Schuitemaker, Evaluation of a mosaic HIV-1 vaccine in a multicentre, randomised, double-blind, placebo-controlled, phase 1/2a clinical trial (APPROACH) and in rhesus monkeys (NHP 13-19). The Lancet 392(10143), 232-43 (2018).
39. A. T. Jones, X. Shen, K. L. Walter, C. C. LaBranche, L. S. Wyatt, G. D. Tomaras, D. C. Montefiori, B. Moss, D. H. Barouch, J. D. Clements, P. A. Kozlowski, R. Varadarajan, R. R. Amara, HIV-1 vaccination by needle-free oral injection induces strong mucosal immunity and protects against SHIV challenge. Nat. Commun. 10, 798 (2019)
40. N. Gao, W. Wang, C. Wang, T. Gu, R. Guo, B. Yu, W. Kong, C. Qin, E. E. Giorgi, Z. Chen, S. Townsley, S. L. Hu, X. Yu, F, Gao, Development of broad neutralization activity in simian/human immunodeficiency virus-infected rhesus macaques after long-term infection. AIDS. 32(5), 555-63 (2018).
41. M. Vaccari, S. N. Gordon, S. Fourati, L. Schifanella, N. P. Liyanage, M. Cameron, B. F. Keele, X. Shen, G. D. Tomaras, E. Billings, M. Rao, A. W. Chung, K. G. Dowell, C. Bailey-Kellogg, E. P. Brown, M. E. Ackerman, D. A. Vargas-Inchaustegui, S. Whitney, M. N. Doster, N. Binello, P. Pegu, D. C. Montefiori, K. Foulds, D. S. Quinn, M. Donaldson, F. Liang, K. Loré, M. Roederer, R. A. Koup, A. McDermott, Z. M. Ma, C. J. Miller, T. B. Phan, D. N. Forthal, M. Blackburn, F. Caccuri, M. Bissa, G. Ferrari, V. Kalyanaraman, M. G. Ferrari, D. Thompson, M. Robert-Guroff, S. Ratto-Kim, J. H. Kim, N. L. Michael, S. Phogat, S. W. Barnett, J. Tartaglia, D. Venzon, D. M. Stablein, G. Alter, R. P. Sekaly, G. Franchini, Adjuvant-dependent innate and adaptive immune signatures of risk of SIVmac251 acquisition. Nat. Med. 22(7), 762-70 (2016).
42. C. D. Pilcher, H. C. Tien, J. J. Jr Eron, P. L. Vernazza, S. Y. Leu, P. W. Stewart, L. E. Goh, M. S. Cohen, Quest Study, Duke-UNC-Emory Acute HIV Consortium, Brief but efficient: acute HIV infection and the sexual transmission of HIV. J. Infect. Dis. 189(10), 1785-92 (2004).
43. M. L. Robb, L. A. Eller, M. Rolland, Acute HIV-1 Infection in Adults in East Africa and Thailand. N. Engl. J. Med. 375(12), 1195 (2016).
44. S. G. Deeks, S. R. Lewin, A. L. Ross, J. Ananworanich, M. Benkirane, P. Cannon, N. Chomont, D. Douek, J. D. Lifson, Y. R. Lo, D. Kuritzkes, D. Margolis, J. Mellors, D. Persaud, J. D. Tucker, F. Barre-Sinoussi; International AIDS Society Towards a Cure Working Group, G. Alter, J. Auerbach, B. Autran, D. H. Barouch, G. Behrens, M. Cavazzana, Z. Chen, É. A. Cohen, G. M. Corbelli, S. Eholié, N. Eyal, S. Fidler, L. Garcia, C. Grossman, G. Henderson, T. J. Henrich, R. Jefferys, H. P. Kiem, J. McCune, K. Moodley, P. A. Newman, M. Nijhuis, M. S. Nsubuga, M. Ott, S. Palmer, D. Richman, A. Saez-Cirion, M. Sharp, J. Siliciano, G. Silvestri, J. Singh, B. Spire, J. Taylor, M. Tolstrup, S. Valente, J. van Lunzen, R. Walensky, I. Wilson, J. Zack, International AIDS Society global scientific strategy: towards an HIV cure. Nat. Med. 22(8), 839-50 (2016).
45. A. Tagarro, M. Chan, P. Zangari, B. Ferns, C. Foster, A. De Rossi, E. Nastouli, M. A. Mũnoz-Fernández, D. Gibb, P. Rossi, C. Giaquinto, A. Babiker, C. Fortuny, R. Freguja, N. Cotugno, A. Judd, A. Noguera-Julian, M. L. Navarro, M. J. Mellado, N. Klein, P. Palma, P. Rojo, EPIICAL Consortium, Early and Highly Suppressive ART are Main Factors Associated with Low Viral Reservoir in European Perinatally HIV Infected Children. J. Acquir Immune Defic. Syndr. 79(2), 269-76 (2018).
46. T. Zhao, The Role of HBZ in HTLV-1-Induced Oncogenesis. Viruses 8(34), 1-12 (2016).

1. A nucleic acid construct which comprises a cDNA molecule encoding a full length antigenomic (+) RNA strand of a measles virus (MeV); and
(i) a first heterologous polynucleotide encoding at least one GAG antigen, a fragment thereof, or a mutated version thereof of a Simian Immunodeficiency Virus (SIV), a Human Immunodeficiency Virus (HIV) or a Human T lymphotropic virus (HTLV), wherein the first heterologous polynucleotide is operatively cloned within an additional transcription unit (ATU) inserted within the cDNA of the antigenomic (+) RNA, in particular an ATU located between the P gene and the M gene of the MeV, in particular in the ATU2 inserted between the P gene and the M gene of the MeV;
(ii) a second heterologous polynucleotide encoding at least one ENV antigen, or a fragment thereof comprising an immunosuppressive domain (ISD), in particular at least one fragment comprising the transmembrane subunit of the ENV antigen, wherein the ENV antigen or its fragment is mutated within its immunosuppressive domain (ISD) and is of a Simian Immunodeficiency Virus (SIV), a Human Immunodeficiency Virus (HIV) or a Human T lymphotropic virus (HTLV), wherein the second heterologous polynucleotide is operatively cloned within the same or a different additional transcription unit (ATU) as in (i) inserted within the cDNA of the antigenomic (+) RNA, in particular an ATU located between the H gene and the L gene of the MeV, in particular in the ATU3 inserted between the H gene and the L gene of the MeV;
wherein the mutation within the ISD domain of ENV reduces the immunosuppressive index of the ENV antigen; and wherein the GAG and ENV antigens, or their respective immunogenic fragments or mutated versions thereof, all originate from the same virus type, in particular are from the same virus strain, more particularly from HIV-1 or HTLV-1.

2. The nucleic acid construct of claim 1, wherein the GAG and ENV antigens are issued from HIV and/or SIV, and which comprises:
(iii) a third heterologous polynucleotide encoding at least one NEF antigen, or a fragment thereof, comprising an immunosuppressive domain (ISD), wherein the NEF antigen is mutated within its ISD domain, and is of a SIV or HIV, wherein the third heterologous polynucleotide is operatively cloned within the same or a different additional transcription unit (ATU) as in (i) or (ii) inserted within the cDNA of the antigenomic (+) RNA, in particular an ATU located upstream the N gene of the MeV, in particular in the ATU1 inserted upstream the N gene of the MeV,
wherein the mutation within the ISD of NEF reduces the immunosuppressive index of the NEF antigen; and wherein the GAG, ENV and NEF antigens, or their respective immunogenic fragments or mutated versions thereof, all originate from the same virus type, in particular are from the same virus strain, more particularly from HIV-1.

3. The nucleic acid construct of claim 1, wherein the GAG and ENV antigens are issued from HTLV, and which comprises:
(iii) a third heterologous polynucleotide encoding at least one HBZ antigen, or a fragment thereof, or a mutated version thereof, and is of HTLV, wherein the third heterologous polynucleotide is operatively cloned within the same or a different additional transcription unit (ATU) as in (i) or (ii) inserted within the cDNA of the antigenomic (+) RNA, in particular an ATU located upstream the N gene of the MeV, in particular in the ATU1 inserted upstream the N gene of the MeV,
wherein the GAG, ENV and HBZ antigens, or their respective immunogenic fragments or mutated versions thereof, all originate from the same virus type, in particular are from the same virus strain, more particularly from HTLV-1.

4. A combination of nucleic acid constructs which comprises:
(a) the first nucleic acid construct according to claim 1 wherein the GAG and ENV antigens are issued from HIV and/or SIV; and
(b) a second nucleic acid construct comprising:
(i′) a second cDNA molecule encoding a full length antigenomic (+) RNA strand of a measles virus (MeV);
(ii′) a third heterologous polynucleotide encoding at least one NEF antigen, or a fragment thereof, mutated within its ISD, of a SIV or HIV, wherein the third heterologous polynucleotide is operatively cloned within an additional transcription unit (ATU) inserted within the cDNA of the antigenomic (+) RNA of (i′), in particular an ATU located upstream the N gene of the MeV, in particular in the ATU1 inserted upstream the N gene of the MeV,
wherein the mutation within the ISD of NEF reduces the immunosuppressive index of the NEF antigen; and wherein the GAG, ENV and NEF antigens or their respective immunogenic fragments or mutated versions thereof, all originate from the same virus type, in particular are from the same virus strain, more particularly from HIV-1.

5. The nucleic acid construct according to claim 1, wherein the first heterologous polynucleotide encodes at least a fragment of an antigen selected from the group consisting of SIV-GAG, SIV-GAGpro, HIV-GAG, HIV-GAGpro, HTLV-GAG, or HTLV-GAGpro, in particular a HIV-1-GAG or HIV-1-GAGpro, in particular an antigen comprising or consisting of the amino acid sequence set forth in SEQ ID No: 1, SEQ ID No: 2, SEQ ID No: 3, SEQ ID No: 4, SEQ ID No: 5, SEQ ID No: 6, SEQ ID No. 45 or SEQ ID No. 46.

6. The nucleic acid construct according to claim 1, wherein the second heterologous polynucleotide encodes at least an antigen or a fragment thereof selected from the group consisting of SIV-ENV, HIV-ENV, or HTLV-ENV, in particular HIV-1-ENV or HTLV-1-ENV, in particular an antigen comprising or consisting of the amino acid sequence set forth in SEQ ID No: 8, SEQ ID No: 10, SEQ ID No: 11, SEQ ID No: 13 or SEQ ID No. 48, or wherein the second heterologous polynucleotide encodes at least a ENV antigen, or a fragment thereof,
wherein said antigen or fragment comprises a mutated immunosuppressive domain (ISD), wherein the mutation corresponds to a substitution or a deletion of at least one amino acid residue within its ISD, as compared to a wild type ENV ISD, in particular as compared to the ISD of the ENV polypeptide of SEQ ID No: 7, SEQ ID No: 9, SEQ ID No: 12, or SEQ ID No. 47.

7. The nucleic acid construct according to claim 2, wherein the third heterologous polynucleotide encodes at least a NEF antigen, or a fragment thereof, comprising or consisting of the amino acid sequence of SIV-NEF or HIV-NEF, in particular HIV-1-NEF, in particular an antigen comprising or consisting of the amino acid sequence set forth in SEQ ID No: 15, SEQ ID No: 17 or SEQ ID No: 19, or wherein the third heterologous polynucleotide encodes at least a NEF antigen, or a fragment thereof, said antigen or fragment comprising a mutated immunosuppressive domain (ISD), wherein the mutation corresponds to a substitution or a deletion of at least one amino acid residue within its ISD, as compared to a wild type NEF IDS, in particular as compared to the ISD of the NEF polypeptide of SEQ ID No: 14, SEQ ID No: 16 or SEQ ID No: 18.

8. The nucleic acid construct according to claim 2, wherein the first heterologous antigen encodes at least a fragment of HIV-GAG or HIV-GAGpro, in particular HIV-1-GAG or HIV-1-GAGpro, in particular a HIV-1-GAG comprising or consisting of the amino acid sequence of SEQ ID No: 2 or HIV-1-GAGpro comprising or consisting of amino acid sequence of SEQ ID No: 5,
wherein the second heterologous polynucleotide encodes ENV or a ENV fragment comprising or consisting of the amino acid sequence of HIV consensus B ENV, or the amino acid sequence of SF162 ENV, in particular the amino acid sequence set forth in the group consisting of SEQ ID No: 20 or SEQ ID No: 21, or wherein the second heterologous polynucleotide encodes at least a fragment of an ENV antigen mutated within its immunosuppressive domain (ISD), wherein the mutation corresponds to a substitution or a deletion of at least one amino acid residue within its ISD, as compared to a wild type ENV ISD, in particular as compared to the ISD of the ENV polypeptide of SEQ ID No: 7, SEQ ID No: 9 or SEQ ID No: 12, in particular at least a fragment of an ENV antigen comprising or consisting of the amino acid sequence of SEQ ID No: 8, SEQ ID No: 10, SEQ ID No: 11 or SEQ ID No: 13, and
wherein the third heterologous polynucleotide encodes at least a fragment of a NEF antigen comprising or consisting of the amino acid sequence of SEQ ID No: 15, SEQ ID No: 17 or SEQ ID No: 19, or wherein the third heterologous polynucleotide encodes at least a fragment of a NEF antigen mutated within its immunosuppressive domain (ISD), wherein the mutation corresponds to a substitution or a deletion of at least one amino acid residue within its ISD, as compared to a wild type NEF ISD, in particular as compared to the ISD of the NEF polypeptide of SEQ ID No: 14, SEQ ID No: 16 or SEQ ID No: 18.

9. The nucleic acid construct according to claim 1, wherein the first heterologous antigen encodes at least a fragment of HTLV-GAG, in particular HTLV-1-GAG, comprising or consisting of the amino acid sequence of SEQ ID No: 45 or HTLV-1-GAGpro comprising or consisting of the amino acid sequence of SEQ ID No: 46, wherein the second heterologous polynucleotide encodes ENV or a ENV fragment comprising or consisting of the amino acid sequence of HTLV ENV, in particular the amino acid sequence set forth in SEQ ID No. 48, or wherein the second heterologous polynucleotide encodes at least a fragment of an ENV antigen mutated within its immunosuppressive domain (ISD), wherein the mutation corresponds to a substitution or a deletion of at least one amino acid residue within its ISD, as compared to a wild type ENV ISD, in particular as compared to the ISD of the ENV polypeptide of SEQ ID No: 47, in particular at least a fragment of an ENV antigen comprising or consisting of the amino acid sequence of SEQ ID No: 48, and
wherein the third heterologous polynucleotide encodes at least a fragment of a HBZ antigen of HTLV comprising or consisting of the amino acid sequence of SEQ ID No. 49, or wherein the third heterologous polynucleotide encodes at least a fragment of a HBZ antigen, wherein HBZ is mutated to reduce its oncogenic properties, in particular wherein the mutation corresponds to a substitution or a deletion of at least one amino acid residue within HBZ, as compared to a wild type HBZ of SEQ ID No: 55, and which is in particular a HBZ antigen associated with at least a fragment of a TAX antigen of the amino acid residue of SEQ ID No. 50.

10. The nucleic acid construct according to claim 1, wherein the measles virus is an attenuated virus strain selected from the group consisting of the Schwarz strain, the Zagreb strain, the AIK-C strain, the Moraten strain, the Philips strain, the Beckenham 4A strain, the Beckenham 16 strain, the Edmonston seed A strain, the Edmonston seed B strain, the CAM-70 strain, the TD 97 strain, the Leningrad-16 strain, the Shanghai 191 strain and the Belgrade strain, in particular the Schwarz strain.

11. The nucleic acid construct according to claim 1, wherein the first nucleic acid construct has a recombinant cDNA sequence selected from the group consisting of:
SEQ ID No: 32 (construct MeV-SIVgag-HIVenv Cons B WT);
SEQ ID No: 40 (construct MeV-SIVgag-HIVenv Cons B MT);
SEQ ID No: 33 (construct MeV-SIVgag-HIVenv SF162 WT);
SEQ ID No: 41 (construct MeV-SIVgag-HIVenv SF162 MT);
SEQ ID No: 43 (construct MeV-SIVgag-HIVenv gp41 WT);
SEQ ID No: 44 (construct MeV-SIVgag-HIVenv gp41 MT); and
SEQ ID No: 54 (construct MeV-HTLVgag-HTLVenv).

12. An infectious recombinant measles virus, said virus comprising in its genome one nucleic acid construct according to claim 1, in particular wherein the infectious replicating measles virus expresses at least one antigen selected from the group consisting of mutated ENV, GAG, or GAGpro, and optionally mutated NEF antigen, or immunogenic fragments thereof.

13. The infectious replicating recombinant measles virus according to claim 12, which elicits a cellular and/or humoral and cellular response, in particular after a prime-boost immunization, more particularly after a homologous prime-boost immunization, against the immunogenic antigen(s) of the GAG, ENV and/or NEF antigens if any, or immunogenic fragments thereof, in particular a T cell response, in particular a IFNγ and/or a IL-2 response.

14. A host cell transfected with the combination of nucleic acid constructs according to claim 4, in particular a mammalian cell, a VERO NK cells, CEF cells, or human embryonic kidney cell line 293T.

15. Recombinant virus like particles (VLPs) comprising a GAG and a ENV antigen, and optionally a NEF antigen or HBZ antigen, or immunogenic fragments thereof, of SIV and/or HIV or HTLV, wherein the antigen or immunogenic fragments thereof are encoded by the first, the second, and optionally the third, heterologous polynucleotides of the nucleic acid constructs according to claim 1.

16. An immunogenic composition, especially a virus vaccine composition, comprising the infectious replicating recombinant measles virus according to claim 12 and a pharmaceutically acceptable vehicle.

17. The composition according to claim 16 for use in the elicitation of a protective, and preferentially prophylactic, immune response against HIV and/or SIV or HTLV by the elicitation of antibodies directed against HIV and/or SIV or HTLV polypeptides or antigenic fragments thereof or mutated version thereof, and/or a cellular or humoral and cellular response against the HIV and/or SIV or HTLV, in a host in need thereof, in particular a human host, in particular a child.

18. The composition of claim 16 for use in the elicitation of a protective, and preferentially prophylactic, immune response against measles virus by the elicitation of antibodies directed against measles virus protein(s), and/or a cellular and/or humoral and cellular response against the measles virus, in a host in need thereof, in particular a human host, in particular a child.

19. A method for preventing or treating a HIV or SIV or HTLV related disease, said method comprising the immunization of a mammalian, especially a human, in particular a child, by the injection, in particular mucosal or intramuscular or subcutaneous injection, more particularly mucosal injection, and most particularly nasal injection, of recombinant Virus Like Particles according to claim 15.


SEQUENCE LISTING (55 sequences)

SEQ ID NO: 1 - protein, 506 aa, Artificial Sequence
  Synthetic sequence
MGARNAVLSGKKADELEKIRLRPGGKKKYMLKHVVWAANELDRFGLAESLLENKEGCQKILSVLAPLVPTGSENLKSLYNTVCVIWCIHAEEKVKHTEEAKQIVQRHLVVETGTAETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLRLQKCVRMYNPINILDVKQRPKEPFQSYVDRFYKSLRAEQTDAAVKNWMTQTLLIQNANPDCKLVLKGLGVNPTLEEMLTACQGVGGPGQKARLMAEALKEALRPVPTPFAAAQQRGPRKPIKCWNCGKEGHSARQCRAPRRQRCWKCGKMDHVMAKCPDRQAGFLGLGPWGKKPRNFPMAQVHQGLTPTAPPEDPAVDLLKNYMQLGKQQRESREKPYKEVTEDLLHLNSLFGGDQ

SEQ ID NO: 2 - protein, 500 aa, Artificial Sequence
  Synthetic sequence
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 3 - protein, 521 aa, Artificial Sequence
  Synthetic sequence
MGARGSVLSGKKTDELEKVRLRPGGKKKYMLKHVVWAVNELDRFGLAESLLESKEGCQKILKVLAPLVPTGSENLKSLFNIVCVIFCLHAEEKVKDTEEAKKIAQRHLAADTEKMPATNKPTAPPSGGNYPVQQLAGNYVHLPLSPRTLNAWVKLVEEKKFGAEVVPGFQALSEGCTPYDINQMLNCVGEHQAAMQIIREIINEEAADWDQQHPSPGPMPAGQLRDPRGSDIAGTTSTVEEQIQWMYRAQNPVPVGNIYRRWIQLGLQKCVRMYNPTNILDIKQGPKEPFQSYVDRFYKSLRAEQTDPAVKNWMTQTLLIQNANPDCKLVLKGLGMNPTLEEMLTACQGIGGPGQKARLMAEALKEALTPAPIPFAAVQQKAGKRGTVTCWNCGKQGHTARQCRAPRRQGCWKCGKTGHIMSKCPERQAGFLGLGPWGKKPRNFPMTQVPQGVTPSAPPMNPAEGMTPRGATPSAPPADPAVEMLKSYMQMGRQQRESRERPYKEVTEDLLHLNSLFGEDQ

SEQ ID NO: 4 - protein, 1448 aa, Artificial Sequence
  Synthetic sequence
MGARNSVLSGKKADELEKIRLRPGGKKKYMLKHVVWAANELDRFGLAESLLENKEGCQKILSVLAPLVPTGSENLKSLYNTVCVIWCIHAEEKVKHTEEAKQIVQRHLVMETGTAETMPKTSRPTAPFSGRGGNYPVQQIGGNYTHLPLSPRTLNAWVKLIEEKKFGAEVVSGFQALSEGCLPYDINQMLNCVGDHQAAMQIIRDIINEEAADWDLQHPQQAPQQGQLREPSGSDIAGTTSTVEEQIQWMYRQQNPIPVGNIYRRWIQLGLQKCVRMYNPTNILDVKQGPKEPFQSYVDRFYKSLRAEQTDPAVKNWMTQTLLIQNANPDCKLVLKGLGTNPTLEEMLTACQGVGGPGQKARLMAEALKEALAPAPIPFAAAQQKGPRKPIKCWNCGKEGHSARQCRAPRRQGCWKCGKMDHVMAKCPNRQAGFFRPWPLGKEAPQFPHGSSASGADANCSPRRTSCGSAKELHALGQAAERKQREALQGGDRGFAAPQFSLWRRPVVTAHIEGQPVEVLLDTGADDSIVTGIELGPHYTPKIVGGIGGFINTKEYKNVEIEVLGKRIKGTIMTGDTPINIFGRNLLTALGMSLNLPIAKVEPVKSPLKPGKDGPKLKQWPLSKEKIVALREICEKMEKDGQLEEAPPTNPYNTPTFAIKKKDKNKWRMLIDFRELNRVTQDFTEVQLGIPHPAGLAKRKRITVLDIGDAYFSIPLDEEFRQYTAFTLPSVNNAEPGKRYIYKVLPQGWKGSPAIFQYTMRHVLEPFRKANPDVTLVQYMDDILIASDRTDLEHDRVVLQLKELLNSIGFSSPEEKFQKDPPFQWMGYELWPTKWKLQKIELPQRETWTVNDIQKLVGVLNWAAQIYPGIKTKHLCRLIRGKMTLTEEVQWTEMAEAEYEENKIILSQEQEGCYYQESKPLEATVIKSQDNQWSYKIHQEDKILKVGKFAKIKNTHTNGVRLLAHVIQKIGKEAIVIWGQVPKFHLPVEKDVWEQWWTDYWQVTWIPEWDFISTPPLVRLVFNLVKDPIEGEETYYVDGSCSKQSKEGKAGYITDRGKDKVKVLEQTTNQQAELEAFLMALTDSGPKANIIVDSQYVMGIITGCPTESESRLVNQIIEEMIKKTEIYVAWVPAHKGIGGNQEIDHLVSQGIRQVLFLEKIEPAQEEHSKYHSNIKELVFKFGLPRLVAKQIVDTCDKCHQKGEAIHGQVNSDLGTWQMDCTHLEGKIVIVAVHVASGFIEAEVIPQETGRQTALFLLKLASRWPITHLHTDNGANFASQEVKMVAWWAGIEHTFGVPYNPQSQGVVEAMNHHLKNQIDRIREQANSVETIVLMAVHCMNFKRRGGIGDMTPAERLINMITTEQEIQFQQSKNSKFKNFRVYYREGRDQLWKGPGELLWKGEGAVILKVGTDIKVVPRRKAKIIKDYGGGKEMDSSSHMEDTGEAREVA

SEQ ID NO: 5 - protein, 1435 aa, Artificial Sequence
  Synthetic sequence
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 6 - protein, 1465 aa, Artificial Sequence
  Synthetic sequence
MGARGSVLSGKKTDELEKVRLRPGGKKKYMLKHVVWAVNELDRFGLAESLLESKEGCQKILKVLAPLVPTGSENLKSLFNIVCVIFCLHAEEKVKDTEEAKKIAQRHLAADTEKMPATNKPTAPPSGGNYPVQQLAGNYVHLPLSPRTLNAWVKLVEEKKFGAEVVPGFQALSEGCTPYDINQMLNCVGEHQAAMQIIREIINEEAADWDQQHPSPGPMPAGQLRDPRGSDIAGTTSTVEEQIQWMYRAQNPVPVGNIYRRWIQLGLQKCVRMYNPTNILDIKQGPKEPFQSYVDRFYKSLRAEQTDPAVKNWMTQTLLIQNANPDCKLVLKGLGMNPTLEEMLTACQGIGGPGQKARLMAEALKEALTPAPIPFAAVQQKAGKRGTVTCWNCGKQGHTARQCRAPRRQGCWKCGKTGHIMSKCPERQAGFLRVRTLGKEASQLPHDPSASGSDTICTPDEPSRGHDTSGGDTICAPCRSSSGDAEKLHADGETTEREPRETLQGGDRGFAAPQFSLWRRPVVKACIEGQSVEVLLDTGVDDSIVAGIELGSNYTPKIVGGIGGFINTKEYKDVEIEVVGKRVRATIMTGDTPINIFGRNILNTLGMTLNFPVAKVEPVKVELKPGKDGPKIRQWPLSREKILALKEICEKMEKEGQLEEAPPTNPYNTPTFAIKKKDKNKWRMLIDFRELNKVTQDFTEVNWVFPTRQVAEKRRITVIDVGDAYFSIPLDPNFRQYTAFTLPSVNNAEPGKRYIYKVLPQGWKGSQSICQYSMRKVLDPFRKANSDVIIIQYMDDILIASDRSDLEHDRVVSQLKELLNDMGFSTPEEKFQKDPPFKWMGYELWPKKWKLQKIQLPEKEVWTVNAIQKLVGVLNWAAQLFPGIKTRHICKLIRGKMTLTEEVQWTELAEAELQENKIILEQEQEGSYYKERVPLEATVQKNLANQWTYKIHQGNKVLKVGKYAKVKNTHTNGVRLLAHVVQKIGKEALVIWGEIPVFHLPVERETWDQWWTDYWQVTWIPEWDFVSTPPLIRLAYNLVKDPLEGRETYYTDGSCNRTSKEGKAGYVTDRGKDKVKVLEQTTNQQAELEAFALALTDSEPQVNIIVDSQYVMGIIAAQPTETESPIVAKIIEEMIKKEAVYVGWVPAHKGLGGNQEVDHLVSQGIRQVLFLEKIEPAQEEHEKYHGNVKELVHKFGIPQLVAKQIVNSCDKCQQKGEAIHGQVNADLGTWQMDCTHLEGKIIIVAVHVASGFIEAEVIPQETGRQTALFLLKLASRWPITHLHTDNGANFTSPSVKMVAWWVGIEQTFGVPYNPQSQGVVEAMNHHLKNQIDRLRDQAVSIETVVLMATHCMNFKRRGGIGDMTPAERLVNMITTEQEIQFFQAKNLKFQNFQVYYREGRDQLWKGPGELLWKGEGAVIIKVGTEIKVVPRRKAKIIRHYGGGKGLDCSADMEDTRQAREMAQSD

SEQ ID NO: 7 - protein, 878 aa, Artificial Sequence
  Synthetic sequence
  misc_feature (733)..(733): Xaa can be any naturally occurring amino acid
MGCLGNQLLIAILLLSVYGIYCTQYVTVFYGVPAWRNATIPLFCATKNRDTWGTTQCLPDNGDYSELALNVTESFDAWENTVTEQAIEDVWQLFETSIKPCVKLSPLCITMRCNKSETDRWGLTKSSTTITTAAPTSAPVSEKIDMVNETSSCIAQNNCTGLEQEQMISCKFTMTGLKRDKTKEYNETWYSTDLVCEQGNSTDNESRCYMNHCNTSVIQESCDKHYWDTIRFRYCAPPGYALLRCNDTNYSGFMPKCSKVWSSCTRMMETQTSTWFGFNGTRAENRTYIYWHGRDNRTIISLNKYYNLTMKCRRPGNKTVLPVTIMSGLVFHSQPINDRPKQAWCWFGGKWKDAIKEVKQTIVKHPRYTGTNNTDKINLTAPGGGDPEVTFMWTNCRGEFLYCKMNWFLNWVEDRDVTTQRPKERHRRNYVPCHIRQIINTWHKVGKNVYLPPREGDLTCNSTVTSLIANIDWTDGNQTSITMSAEVAELYRLELGDYKLVEITPIGLAPTDVKRYTTGGTSRNKRGVFVLGFLGFLATAGSAMGAASLTLTAQSRTLLAGIVQQQQQLLDWKRQQELLRLTVWGTKNLQTRVTAIEKYLKDQAQLNAWGCAFRQVCHTTVPWPNASLTPDWNNDTWQEWERKVDFLEENITALLEEAQIQQEKNMYELQKLNSWDVFGNWFDLASWIKYIQYGIYVWGVILLRIVIYIVQMLAKLRQGYRPVFSSPPSYFQXTHTQQDPALPTREGKEGDGGEGGGNSSWPWQIEYIHFLIRQLIRLLTWLFSNCRTLLSRAYQILQPILQRLSATLRRVREVLRTELTYLQYGWSYFHEAVQAGWRSATETLAGAWRDLWETLRRGGRWILAIPRRIRQGLELTLL

SEQ ID NO: 8 - protein, 878 aa, Artificial Sequence
  Synthetic sequence
  misc_feature (733)..(733): Xaa can be any naturally occurring amino acid
MGCLGNQLLIAILLLSVYGIYCTQYVTVFYGVPAWRNATIPLFCATKNRDTWGTTQCLPDNGDYSELALNVTESFDAWENTVTEQAIEDVWQLFETSIKPCVKLSPLCITMRCNKSETDRWGLTKSSTTITTAAPTSAPVSEKIDMVNETSSCIAQNNCTGLEQEQMISCKFTMTGLKRDKTKEYNETWYSTDLVCEQGNSTDNESRCYMNHCNTSVIQESCDKHYWDTIRFRYCAPPGYALLRCNDTNYSGFMPKCSKVWSSCTRMMETQTSTWFGFNGTRAENRTYIYWHGRDNRTIISLNKYYNLTMKCRRPGNKTVLPVTIMSGLVFHSQPINDRPKQAWCWFGGKWKDAIKEVKQTIVKHPRYTGTNNTDKINLTAPGGGDPEVTFMWTNCRGEFLYCKMNWFLNWVEDRDVTTQRPKERHRRNYVPCHIRQIINTWHKVGKNVYLPPREGDLTCNSTVTSLIANIDWTDGNQTSITMSAEVAELYRLELGDYKLVEITPIGLAPTDVKRYTTGGTSRNKRGVFVLGFLGFLATAGSAMGAASLTLTAQSRTLLAGIVQQQQQLLDWKRQQELLRLTVWGTKNLQTRVTAIEKYRKDQAQLNAWGCAFRQVCHTTVPWPNASLTPDWNNDTWQEWERKVDFLEENITALLEEAQIQQEKNMYELQKLNSWDVFGNWFDLASWIKYIQYGIYVWGVILLRIVIYIVQMLAKLRQGYRPVFSSPPSYFQXTHTQQDPALPTREGKEGDGGEGGGNSSWPWQIEYIHFLIRQLIRLLTWLFSNCRTLLSRAYQILQPILQRLSATLRRVREVLRTELTYLQYGWSYFHEAVQAGWRSATETLAGAWRDLWETLRRGGRWILAIPRRIRQGLELTLL

SEQ ID NO: 9 - protein, 858 aa, Artificial Sequence
  Synthetic sequence
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLGNATNTNSSNNSSSGEMMMEKGEIKNCSFNISTSIRGKVQKEYAFFYKLDIIPIDNDTTSYTLTSCNTSVITQACPKVSFEPIPIHYCAPAGFAILKCNNKTFNGTGPCTNVSTVQCTHGIRPWSTQLLLNGSLAEEEVVIRSANFTDNAKTIIVQLNQSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKIFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEWQGACRAIRHIPRRIRQGLERILL

SEQ ID NO: 10 - protein, 858 aa, Artificial Sequence
  Synthetic sequence
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLGNATNTNSSNNSSSGEMMMEKGEIKNCSFNISTSIRGKVQKEYAFFYKLDIIPIDNDTTSYTLTSCNTSVITQACPKVSFEPIPIHYCAPAGFAILKCNNKTFNGTGPCTNVSTVQCTHGIRPWSTQLLLNGSLAEEEVVIRSANFTDNAKTIIVQLNQSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHLLQLTVWGIKQLQARILAVERRLKDQQLLGIWGCSGKLICTTAVPWNASWSNKSLEQIWNNMTWMEWDREINNYTSLIHSLIEESQNQQEKNEQELLELDKWASLWNWFNITNWLWYIKIFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEWQGACRAIRHIPRRIRQGLERILL

SEQ ID NO: 11 - protein, 859 aa, Artificial Sequence
  Synthetic sequence
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLGNATNTNSSNTNSSSGEMMMEKGEIKNCSFNISTSIRGKVQKEYAFFYKLDIIPIDNDTTSYTLTSCNTSVITQACPKVSFEPIPIHYCAPAGFAILKCNNKTFNGTGPCTNVSTVQCTHGIRPWSTQLLLNGSLAEEEVVIRSANFTDNAKTIIVQLNQSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGARSMTLTVQARQLLSGIVQQQNNLLRAIEAQQHLLQLTVWGIKQLQARILAVERRLKDQQLLGIWGCSGKLICTTAVPWNASWSNASLEQIWNNMTWMEWDREINNYTSLIHSLIEESQNQQEKNEQELLELDKWASLWNWFNITNWLWYIKIFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEWQGACRAIRHIPRRIRQGLERILL

SEQ ID NO: 12 - protein, 856 aa, Artificial Sequence
  Synthetic sequence
MEPGRNQLFWILLTSACLVYCSQYVTVFYGIPAWKNASIPLFCATKNRDTWGTIQCLPDNDDYQEIILNVTEAFDAWNNTVTEQAVEDVWHLFETSIKPCVKLTPLCVAMNCSRVQGNTTTPNPRTSSSTTSRPPTSAASIINETSNCIENNTCAGLGYEEMMQCEFNMKGLEQDKKRRYKDTWYLEDWCDNTTAGTCYMRHCNTSIIKESCDKHYWDAMRFRYCAPPGFALLRCNDTNYSGFEPKCTKVVAASCTRMMETQTSTWFGFNGTRAENRTYIYWHGRDNRTIISLNKYYNLTMRCKRPGNKTVLPITLMSGLVFHSQPINTRPRQAWCRFGGRWREAMQEVKQTLVQHPRYKGINDTGKINFTKPGAGSDPEVAFMWTNCRGEFLYCNMTWFLNWVEDKNQTRRNYCHIKQIINTWHKVGKNVYLPPREGELACESTVTSIIANIDIDKNRTHTNITFSAEVAELYRLELGDYKLIEITPIGFAPTDQRRYSSTPVRNKRGVFVLGFLGFLATAGSAMGARSLTLSAQSRTLLAGIVQQQQQLLDWKRQQEMLRLTVWGTKNLQARVTAIEKYLKHQAQLNSWGCAFRQVCHTTVPWVNDSLSPDWKNMTWQEWEKQVRYLEANISQSLEEAQIQQEKNMYELQKLNSWDILGNWFDLTSWVKYIQYGVHIWGIIALRIAIYVVQLLSRFRKGYRPVFSSPPGYLQQIHIHKDRGQPANEGTEEDVGGDSGYDLWPWPINYVQFLIHLLTRLLIGLYNICRDLLSKNSPTRRLISQSLTAIRDWLRLKAAQLQYGCEWIQEAFQAFARTTRETLAGAWGWLWEAARRIGRGILAVPRRIRQGAELALL

SEQ ID NO: 13 - protein, 856 aa, Artificial Sequence
  Synthetic sequence
MEPGRNQLFWILLTSACLVYCSQYVTVFYGIPAWKNASIPLFCATKNRDTWGTIQCLPDNDDYQEIILNVTEAFDAWNNTVTEQAVEDVWHLFETSIKPCVKLTPLCVAMNCSRVQGNTTTPNPRTSSSTTSRPPTSAASIINETSNCIENNTCAGLGYEEMMQCEFNMKGLEQDKKRRYKDTWYLEDWCDNTTAGTCYMRHCNTSIIKESCDKHYWDAMRFRYCAPPGFALLRCNDTNYSGFEPKCTKVVAASCTRMMETQTSTWFGFNGTRAENRTYIYWHGRDNRTIISLNKYYNLTMRCKRPGNKTVLPITLMSGLVFHSQPINTRPRQAWCRFGGRWREAMQEVKQTLVQHPRYKGINDTGKINFTKPGAGSDPEVAFMWTNCRGEFLYCNMTWFLNWVEDKNQTRRNYCHIKQIINTWHKVGKNVYLPPREGELACESTVTSIIANIDIDKNRTHTNITFSAEVAELYRLELGDYKLIEITPIGFAPTDQRRYSSTPVRNKRGVFVLGFLGFLATAGSAMGARSLTLSAQSRTLLAGIVQQQQQLLDWKRQQEMLRLTVWGTKNLQARVTAIEKYRKHQAQLNSWGCAFRQVCHTTVPWVNDSLSPDWKNMTWQEWEKQVRYLEANISQSLEEAQIQQEKNMYELQKLNSWDILGNWFDLTSWVKYIQYGVHIWGIIALRIAIYVVQLLSRFRKGYRPVFSSPPGYLQQIHIHKDRGQPANEGTEEDVGGDSGYDLWPWPINYVQFLIHLLTRLLIGLYNICRDLLSKNSPTRRLISQSLTAIRDWLRLKAAQLQYGCEWIQEAFQAFARTTRETLAGAWGWLWEAARRIGRGILAVPRRIRQGAELALL

SEQ ID NO: 14 - protein, 263 aa, Artificial Sequence
  Synthetic sequence
MGGAISMRRSRPSGDLRQRLLRARGETYGRLLGEVEDGYSQSPGGLDKGLSSLSCEGQKYNQGQYMNTPWRNPAEEREKLAYRKQNMDDIDEEDDDLVGVSVRPKVPLRTMSYKLAIDMSHFIKEKGGLEGIYYSARRHRILDIYLEKEEGIIPDWQDYTSGPGIRYPKTFGWLWKLVPVNVSDEAQEDEEHYLMHPAQTSQWDDPWGEVLAWKFDPTLAYTYEAYVRYPEEFGSKSGLSEEEVRRRLTARGLLNMADKKETR

SEQ ID NO: 15 - protein, 263 aa, Artificial Sequence
  Synthetic sequence
MGGAISMRRSRPSGDLRQRLLRARGETYGRLLGEVEDGYSQSPGGLDKGLSSLSCEGQKYNQGQYMNTPWRNPAEEREKLAYRKQNMDDIDEEDDDLVGVSVRPKVPLRTMSYKLAIDMSHFIKRKGGLEGIYYSARRHRILDIYLEKEEGIIPDWQDYTSGPGIRYPKTFGWLWKLVPVNVSDEAQEDEEHYLMHPAQTSQWDDPWGEVLAWKFDPTLAYTYEAYVRYPEEFGSKSGLSEEEVRRRLTARGLLNMADKKETR

SEQ ID NO: 16 - protein, 206 aa, Artificial Sequence
  Synthetic sequence
MGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 17 - protein, 206 aa, Artificial Sequence
  Synthetic sequence
MGGKWSKSSVVGWPTVRERMRRAEPAADGVGAASRDLEKHGAITSSNTAATNAACAWLEAQEEEEVGFPVTPQVPLRPMTYKAAVDLSHFLKRKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 18 - protein, 180 aa, Artificial Sequence
  Synthetic sequence
MGASGSKKRSKPLQGLQERLLQARGETCGGRCNESGGGYLQSHEGSGREQNSPSCEGQRYQQGDFVNTPWRTPAAEREKELYKQQNMDDVDLDDDDQVGFPVTPRVPLRPMTFKLAVDMSHFIKEKGGLEGLFYSQRRHRILDLYLDKAFTLYPEEFGHNSGLPEKEWKARLKARGIPFS

SEQ ID NO: 19 - protein, 180 aa, Artificial Sequence
  Synthetic sequence
MGASGSKKRSKPLQGLQERLLQARGETCGGRCNESGGGYLQSHEGSGREQNSPSCEGQRYQQGDFVNTPWRTPAAEREKELYKQQNMDDVDLDDDDQVGFPVTPRVPLRPMTFKLAVDMSHFIKRKGGLEGLFYSQRRHRILDLYLDKAFTLYPEEFGHNSGLPEKEWKARLKARGIPFS

SEQ ID NO: 20 - protein, 847 aa, Artificial Sequence
  Synthetic sequence
MRVKGIRKNYQHLWRGGTLLLGMLMICSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKRAVTLGAMFLGFLGAAGSTMGARSLTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYIKIFIMIVGGLVGLRIVFTVLSIVNRVRQGYSPLSFQTRFPAPRGPDRPEGIEEEGGERDRDRSSPLVHGLLALIWDDLRSLCLFSYHRLRDLILIAARIVELLGRRGWEALKYWGNLLQYWIQELKNSAVSLFDAIAIAVAEGTDRIIEVAQRIGRAFLHIPRRIRQGFERALL

SEQ ID NO: 21 - protein, 849 aa, Artificial Sequence
  Synthetic sequence
MKVMGTKKNYQHLWRWGIMLLGMLMMSSAAEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREGMKNCSFNTTTVIRDKIQKEYALFYKLDIVPIEGKNTNTSYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIIGGLVGLRTVCAVLSIVNRVRQGYSPLSFQTRLPNPRGPGRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 22 - protein, 231 aa, Artificial Sequence
  Synthetic sequence
METDTLLLWVLLLWVPGSTGDAAQPAGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 23 - protein, 231 aa, Artificial Sequence
  Synthetic sequence
METDTLLLWVLLLWVPGSTGDAAQPAGGKWSKSSVVGWPTVRERMRRAEPAADGVGAASRDLEKHGAITSSNTAATNAACAWLEAQEEEEVGFPVTPQVPLRPMTYKAAVDLSHFLKRKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 24 - DNA, 115 bp, Artificial Sequence
  Synthetic sequence
cttaggaaccaggtccacacagccgccagcccatcaacgcgtacgatgtaggcgcgcagcgcttagacgtctcgcgatcgatactagtacaacctaaatccattataaaaaactt

SEQ ID NO: 25 - DNA, 18967 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 26 - protein, 284 aa, Artificial Sequence
  Synthetic sequence
METDTLLLWVLLLWVPGSTGDAGGAISMRRSRPSGDLRQRLLRARGETYGRLLGEVEDGYSQSPGGLDKGLSSLSCEGQKYNQGQYMNTPWRNPAEEREKLAYRKQNMDDIDEEDDDLVGVSVRPKVPLRTMSYKLAIDMSHFIKEKGGLEGIYYSARRHRILDIYLEKEEGIIPDWQDYTSGPGIRYPKTFGWLWKLVPVNVSDEAQEDEEHYLMHPAQTSQWDDPWGEVLAWKFDPTLAYTYEAYVRYPEEFGSKSGLSEEEVRRRLTARGLLNMADKKETR

SEQ ID NO: 27 - protein, 284 aa, Artificial Sequence
  Synthetic sequence
METDTLLLWVLLLWVPGSTGDAGGAISMRRSRPSGDLRQRLLRARGETYGRLLGEVEDGYSQSPGGLDKGLSSLSCEGQKYNQGQYMNTPWRNPAEEREKLAYRKQNMDDIDEEDDDLVGVSVRPKVPLRTMSYKLAIDMSHFIKRKGGLEGIYYSARRHRILDIYLEKEEGIIPDWQDYTSGPGIRYPKTFGWLWKLVPVNVSDEAQEDEEHYLMHPAQTSQWDDPWGEVLAWKFDPTLAYTYEAYVRYPEEFGSKSGLSEEEVRRRLTARGLLNMADKKETR

SEQ ID NO: 28 - protein, 227 aa, Artificial Sequence
  Synthetic sequence
METDTLLLWVLLLWVPGSTGDAGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 29 - protein, 227 aa, Artificial Sequence
  Synthetic sequence
METDTLLLWVLLLWVPGSTGDAGGKWSKSSVVGWPTVRERMRRAEPAADGVGAASRDLEKHGAITSSNTAATNAACAWLEAQEEEEVGFPVTPQVPLRPMTYKAAVDLSHFLKRKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 30 - protein, 271 aa, Artificial Sequence
  Synthetic sequence
METDTLLLWVLLLWVPGSTGDAGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCGGGGSGSPNKGSGTTSGTTRLLSGHTCFTLTGLLGTLVTMGLLT

SEQ ID NO: 31 - protein, 271 aa, Artificial Sequence
  Synthetic sequence
METDTLLLWVLLLWVPGSTGDAGGKWSKSSVVGWPTVRERMRRAEPAADGVGAASRDLEKHGAITSSNTAATNAACAWLEAQEEEEVGFPVTPQVPLRPMTYKAAVDLSHFLKRKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCGGGGSGSPNKGSGTTSGTTRLLSGHTCFTLTGLLGTLVTMGLLT

SEQ ID NO: 32 - DNA, 23075 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgggccaccatgggggtgaggaacagcgtgctgagcgggaagaaggccgatgagctggagaagatcaggctgaggcccaacgggaagaagaagtacatgctgaagcacgtggtgtgggccgccaacgagctggataggttcgggctggccgagagcctgctggagaacaaggaggggtgccagaagatcctgagcgtgctggcccccctggtgcccacagggagcgagaacctgaagagcctgtacaacacagtgtgcgtgatctggtgcatccacgccgaggagaaggtgaagcacacagaggaggccaagcagatcgtgcagaggcacctggtggtggagacagggacaacagagacaatgcccaagacaagcaggcccacagccccctccagcgggaggggcgggaactaccccgtgcagcagatcgggggcaactacgtgcacctgcccctgagccccaggacactgaacgcctgggtgaagctgatcgaggagaagaagttcggggccgaggtggtgcccgggttccaggccctgagcgaggggtgcacaccctacgatatcaaccagatgctgaactgcgtgggggatcaccaggccgccatgcagatcatcagggatatcatcaacgaggaggccgccgattgggatctgcaacacccacagcccgctccccaacaggggcagctgagggagcccagcgggagcgatatcgccgggacaacaagcagcgtggatgagcagatccagtggatgtacaggcagcagaaccccatccccgtcgggaacatctacaggaggtggatccagctggggctgcagaagtgcgtgaggatgtacaaccccacaaacatcctggatgtgaagcaggggccaaaggagccattccagagctacgtcgataggttctacaagagcctgagggccgagcagacagatgccgccgtgaagaactggatgacacagacactgctgatccagaacgccaaccccgattgcaagctggtgctgaaaggcctgggggtgaatcccacactggaggagatgttgacagcctgccagggggtggggggacccgggcagaaagccaggctgatggccgaagccctgaaggaagctctggctcccgtgcccatcccattcgccgctgcccagcagagggggcccaggaagcccatcaagtgctggaactgcgggaaggaggggcacagcgccaggcagtgcagggcccccaggaggcaggggtgctggaagtgcgggaagatggatcacgtgatggccaagtgccccgataggcaggccgggttcctggggctggggccctggggcaagaagcccaggaactttccaatggcccaggtgcaccaggggctgatgcccacagctccccccgaggatcccgccgtggatctgctgaagaactacatgcagctcgggaagcaacagagggagaagcagagggagagcagggagaagccctacaaggaggtgacagaggatttgctgcacctgaacagcctgttcgggggggatcagtgatagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacgggccaccatgcgcgtgaagggcatccgcaagaactaccagcacctgtggcgctggggcaccatgctgctgggcatgctgatgatctgcagcgccgccgagcagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggaggtggtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaacaccagcgtgatcacccaggcctgccccaaagtgagcttcgagcccatccccatccactactgcgcccccgccggcttcgccatcctgaagtgcaacgacaagaagttcaacggcaccggcccctgcaccaacgtgagcaccgtgcagtgcacccacggcatccgccccgtggtgagcacccagctgctgctgaacggcagcctggccgaggaggaggtggtgatccgcagcgagaacttcaccgacaacgccaagaccatcatcgtgcagctgaacgagagcgtggagatcaactgcacccgccccaacaacaacacccgcaagagcatcaacatcggccccggccgcgccctgtacaccaccggcgagatcatcggcgacatccgccaggcccactgcaacatcagccgcgccaagtggaacaacaccctgaagcagatcgtgatcaagctgcgcgagcagttcggcaacaagaccatcgtgttcaaccagagcagcggcggcgaccccgagatcgtgatgcacagcttcaactgcggcggcgagttcttctactgcaacagcacccagctgttcacctggaacgacacccgcaagctgaacaacaccggccgcaacatcaccctgccctgccgcatcaagcagatcatcaacatgtggcaggaagtgggcaaggccatgtacgcccctcccatccgcggccagatccgctgcagcagcaacatcaccggcctgctgctgacccgcgacggcggcaaggacaccaacggcaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgggccgtgggcatcggcgccatgttcctgggcttcctgggcgccgccggcagcaccatgggcgccgccagcatgaccctgaccgtgcaggcccgccagctgctgagcggcatcgtgcagcagcagaacaacctgctgcgggccatcgaggcccagcagcacctgctgcagctgaccgtgtggggcatcaagcagctgcaggcccgcgtgctggccgtggagcgctacctgaaggaccagcagctgctgggcatctggggctgcagcggcaagctgatctgcaccaccgccgtgccctggaacgccagctggagcaacaagagcctggaccagatctggaacaacatgacctggatggagtgggagcgcgagatcgacaactacaccagcctgatctacaccctgatcgaggagagccagaaccagcaggagaagaacgagcaggagctgctggagctggacaagtgggccagcctgtggaactggttcgacatcaccaactggctgtggtacatcaagatcttcatcatgatcgtgggcggcctggtgggcctgcgcatcgtgttcgccgtgctgagcatcgtgaaccgcgtgcgccagggctacagccccctgagcttccagacccgcctgcccgccccccgcggccccgaccgccccgagggcatcgaggaggagggcggcgagcgcgaccgcgaccgcagcggccgcctggtggacggcttcctggccctgatctgggtggacctgcgcagcctgtgcctgttcagctaccaccgcctgcgcgacctgctgctgatcgtgacccgcatcgtggagctgctgggccgccgcggctgggaggccctgaagtactggtggaacctgctgcagtactggagccaggagctgaagaacagcgccgtgagcctgctgaacgccaccgccatcgccgtggccgagggcaccgaccgcgtgatcgaggtggtgcagcgggcctgccgcgccatcctgcacatcccccgccgcatccgccagggcctggagcgggccctgctgtaatagcgcgcagcgctgacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 33 - DNA, 20174 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagctagcctaccctccatcattgttataaaaaacttaggaaccaggtccacacagccgccagcccatcaacgcgtacggccaccatggagaccgacaccctgctgttgtgggtgctgctgctgtgggtccccggcagcacaggcgatgccggcggcgctatcagcatgaggcggtctcggcctagcggcgatctgaggcagcggctgctgcgggccaggggcgagacctacggcagactgctgggcgaggtggaggacggctacagccagagccccggcggcctggacaaaggattgagcagcctgagctgcgagggccagaagtacaaccagggccagtacatgaacaccccctggaggaaccccgccgaggagcgggagaagctggcctaccggaagcagaacatggacgacatcgacgaggaggatgacgacctggtgggcgtgagcgtgcggcccaaggtgcccctgcggaccatgagctacaagctggctatcgacatgagccacttcatcaaggagaaaggaggcctggagggcatctactacagcgccaggcggcaccggatcctggacatctacctggagaaggaggaaggcatcatccccgactggcaggactacaccagcggccccggcatccggtaccccaagaccttcggctggctgtggaagctggtgcccgtgaacgtgagcgacgaggcccaggaggacgaagagcactacctgatgcaccccgcccagaccagccagtgggacgacccctggggggaggtgctggcctggaagttcgaccccaccttggcctacacctacgaggcctacgtgaggtaccccgaggagttcggcagcaagagcggcctgagcgaagaggaagtgaggcggcggctgacagctcggggcctgctgaacatggccgacaagaaggagacccggtgatgagcgcgcagcgcttagacgtctcgcgatcgatatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccgccaccgcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccagtaatagcgcgcagcgctgacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 34 - protein, 39 aa, Artificial Sequence
  Synthetic sequence
GSPNKGSGTTSGTTRLLSGHTCFTLTGLLGTLVTMGLLT

SEQ ID NO: 35 - DNA, 120 bp, Artificial Sequence
  Synthetic sequence
ggatccccaaataaaggaagtggaaccacttcaggtactacccgtcttctatctgggcacacgtgtttcacgttgacaggtttgcttgggacgctagtaaccatgggcttgctgacttaa

SEQ ID NO: 36 - protein, 20 aa, Artificial Sequence
  Synthetic sequence
METDTLLLWVLLLWVPGSTG

SEQ ID NO: 37 - DNA, 60 bp, Artificial Sequence
  Synthetic sequence
atggagacagacacactcctgctatgggtactgctgctctgggttccaggttccactggt

SEQ ID NO: 38 - protein, 23 aa, Artificial Sequence
  Synthetic sequence
MYRMQLLSCIALSLALVTNSISA

SEQ ID NO: 39 - DNA, 69 bp, Artificial Sequence
  Synthetic sequence
atgtacaggatgcaactcctgtcttgcattgcactaagtcttgcacttgtcacgaattcgatatcggcc

SEQ ID NO: 40 - DNA, 23075 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgggccaccatgggggtgaggaacagcgtgctgagcgggaagaaggccgatgagctggagaagatcaggctgaggcccaacgggaagaagaagtacatgctgaagcacgtggtgtgggccgccaacgagctggataggttcgggctggccgagagcctgctggagaacaaggaggggtgccagaagatcctgagcgtgctggcccccctggtgcccacagggagcgagaacctgaagagcctgtacaacacagtgtgcgtgatctggtgcatccacgccgaggagaaggtgaagcacacagaggaggccaagcagatcgtgcagaggcacctggtggtggagacagggacaacagagacaatgcccaagacaagcaggcccacagccccctccagcgggaggggcgggaactaccccgtgcagcagatcgggggcaactacgtgcacctgcccctgagccccaggacactgaacgcctgggtgaagctgatcgaggagaagaagttcggggccgaggtggtgcccgggttccaggccctgagcgaggggtgcacaccctacgatatcaaccagatgctgaactgcgtgggggatcaccaggccgccatgcagatcatcagggatatcatcaacgaggaggccgccgattgggatctgcaacacccacagcccgctccccaacaggggcagctgagggagcccagcgggagcgatatcgccgggacaacaagcagcgtggatgagcagatccagtggatgtacaggcagcagaaccccatccccgtcgggaacatctacaggaggtggatccagctggggctgcagaagtgcgtgaggatgtacaaccccacaaacatcctggatgtgaagcaggggccaaaggagccattccagagctacgtcgataggttctacaagagcctgagggccgagcagacagatgccgccgtgaagaactggatgacacagacactgctgatccagaacgccaaccccgattgcaagctggtgctgaaaggcctgggggtgaatcccacactggaggagatgttgacagcctgccagggggtggggggacccgggcagaaagccaggctgatggccgaagccctgaaggaagctctggctcccgtgcccatcccattcgccgctgcccagcagagggggcccaggaagcccatcaagtgctggaactgcgggaaggaggggcacagcgccaggcagtgcagggcccccaggaggcaggggtgctggaagtgcgggaagatggatcacgtgatggccaagtgccccgataggcaggccgggttcctggggctggggccctggggcaagaagcccaggaactttccaatggcccaggtgcaccaggggctgatgcccacagctccccccgaggatcccgccgtggatctgctgaagaactacatgcagctcgggaagcaacagagggagaagcagagggagagcagggagaagccctacaaggaggtgacagaggatttgctgcacctgaacagcctgttcgggggggatcagtgatagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacgggccaccatgcgcgtgaagggcatccgcaagaactaccagcacctgtggcgctggggcaccatgctgctgggcatgctgatgatctgcagcgccgccgagcagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggaggtggtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaacaccagcgtgatcacccaggcctgccccaaagtgagcttcgagcccatccccatccactactgcgcccccgccggcttcgccatcctgaagtgcaacgacaagaagttcaacggcaccggcccctgcaccaacgtgagcaccgtgcagtgcacccacggcatccgccccgtggtgagcacccagctgctgctgaacggcagcctggccgaggaggaggtggtgatccgcagcgagaacttcaccgacaacgccaagaccatcatcgtgcagctgaacgagagcgtggagatcaactgcacccgccccaacaacaacacccgcaagagcatcaacatcggccccggccgcgccctgtacaccaccggcgagatcatcggcgacatccgccaggcccactgcaacatcagccgcgccaagtggaacaacaccctgaagcagatcgtgatcaagctgcgcgagcagttcggcaacaagaccatcgtgttcaaccagagcagcggcggcgaccccgagatcgtgatgcacagcttcaactgcggcggcgagttcttctactgcaacagcacccagctgttcacctggaacgacacccgcaagctgaacaacaccggccgcaacatcaccctgccctgccgcatcaagcagatcatcaacatgtggcaggaagtgggcaaggccatgtacgcccctcccatccgcggccagatccgctgcagcagcaacatcaccggcctgctgctgacccgcgacggcggcaaggacaccaacggcaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgggccgtgggcatcggcgccatgttcctgggcttcctgggcgccgccggcagcaccatgggcgccgccagcatgaccctgaccgtgcaggcccgccagctgctgagcggcatcgtgcagcagcagaacaacctgctgcgggccatcgaggcccagcagcacctgctgcagctgaccgtgtggggcatcaagcagctgcaggcccgcgtgctggccgtggagcgccgcctgaaggaccagcagctgctgggcatctggggctgcagcggcaagctgatctgcaccaccgccgtgccctggaacgccagctggagcaacaagagcctggaccagatctggaacaacatgacctggatggagtgggagcgcgagatcgacaactacaccagcctgatctacaccctgatcgaggagagccagaaccagcaggagaagaacgagcaggagctgctggagctggacaagtgggccagcctgtggaactggttcgacatcaccaactggctgtggtacatcaagatcttcatcatgatcgtgggcggcctggtgggcctgcgcatcgtgttcgccgtgctgagcatcgtgaaccgcgtgcgccagggctacagccccctgagcttccagacccgcctgcccgccccccgcggccccgaccgccccgagggcatcgaggaggagggcggcgagcgcgaccgcgaccgcagcggccgcctggtggacggcttcctggccctgatctgggtggacctgcgcagcctgtgcctgttcagctaccaccgcctgcgcgacctgctgctgatcgtgacccgcatcgtggagctgctgggccgccgcggctgggaggccctgaagtactggtggaacctgctgcagtactggagccaggagctgaagaacagcgccgtgagcctgctgaacgccaccgccatcgccgtggccgagggcaccgaccgcgtgatcgaggtggtgcagcgggcctgccgcgccatcctgcacatcccccgccgcatccgccagggcctggagcgggccctgctgtaatagcgcgcagcgctgacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 41 - DNA, 20174 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagctagcctaccctccatcattgttataaaaaacttaggaaccaggtccacacagccgccagcccatcaacgcgtacggccaccatggagaccgacaccctgctgttgtgggtgctgctgctgtgggtccccggcagcacaggcgatgccggcggcgctatcagcatgaggcggtctcggcctagcggcgatctgaggcagcggctgctgcgggccaggggcgagacctacggcagactgctgggcgaggtggaggacggctacagccagagccccggcggcctggacaaaggattgagcagcctgagctgcgagggccagaagtacaaccagggccagtacatgaacaccccctggaggaaccccgccgaggagcgggagaagctggcctaccggaagcagaacatggacgacatcgacgaggaggatgacgacctggtgggcgtgagcgtgcggcccaaggtgcccctgcggaccatgagctacaagctggctatcgacatgagccacttcatcaagcggaaaggaggcctggagggcatctactacagcgccaggcggcaccggatcctggacatctacctggagaaggaggaaggcatcatccccgactggcaggactacaccagcggccccggcatccggtaccccaagaccttcggctggctgtggaagctggtgcccgtgaacgtgagcgacgaggcccaggaggacgaagagcactacctgatgcaccccgcccagaccagccagtgggacgacccctggggggaggtgctggcctggaagttcgaccccaccttggcctacacctacgaggcctacgtgaggtaccccgaggagttcggcagcaagagcggcctgagcgaagaggaagtgaggcggcggctgacagctcggggcctgctgaacatggccgacaagaaggagacccggtgatgagcgcgcagcgcttagacgtctcgcgatcgatatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccgccaccgcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccagtaatagcgcgcagcgctgacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 42 - DNA, 21959 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccgccaccatggggcagatctttagcaggagtgctagcccaattcccagaccacccagggggctggccgcccaccactggctgaactttctgcaggccgcctacaggctggagcccgggcccagcagctacgatttccaccagctgaagaagttcctgaagatcgccctggagacacccgtgtggatctgcccaatcaactacagcctgctggccagcctgctgcccaaaggctaccccgggagggtgaacgagatcctgcacatcctgatccagacacaggctcagatcccaagtaggccagccccaccaccccccagcagccccacacacgatccccccgatagcgatccccagatcccccccccatacgtggagcccacagccccacaggtgctgccagtgatgcatccccacggggccccccccaaccacaggccctggcagatgaaggacctccaggccatcaagcaggaggtgagccaggccgcccccgggagcccccagttcatgcagacaatcaggctggccgtgcagcagttcgatcccacagccaaggatctgcaggatctgctgcagtacctgtgcagcagcctggtggcctccctgcaccaccagcagctggatagcctgatcagcgaggccgagaccagggggatcacagggtacaaccccctggccgggcccctgagggtgcaggccaacaacccacagcagcaggggctgaggagagagtatcagcaactgtggttggctgccttcgccgccctgcccgggagcgccaaggatccaagctgggccagcattctgcaggggctggaggagccctaccacgccttcgtggagaggctgaacatcgccctggataacgggctgcccgaggggacaccaaaggatcccatcctgaggagcctggcctacagcaacgccaacaaggagtgccagaagctgctgcaggccagggggcacacaaacagcccactgggggatatgctgagggcctgccagacatggacacccaaggataagacaaaggtgctggtcgtgcagcccaagaagcccccacccaaccagccctgcttcaggtgcgggaaggccgggcattggagtagagattgtacacaaccaaggccaccacccgggccctgccccctgtgccaggatcccacacactggaagagggattgccccaggctgaagcccacaatccccgagccagagcccgaggaggatgccctgctgctggatctgcccgccgatatcccccaccccaagaacagcatcgggggggaggtgtgatgagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccatgggcaagttcctggccacactgatcctgttcttccagttctgccccctcatcctgggggattacagcccaagctgctgcacactgacaatcggggtgagcagctaccacagcaaaccctgcaaccccgcccagcccgtgtgcagctggacactggatctgctggccctgagcgccgatcaggctctgcagcccccctgccccaacctggtgagctacagctcctaccacgccacatacagcctgtacctgttccctcactggatcaagaagcccaacaggaacgggggggggtactacagcgccagctacagcgatccctgcagcctgaagtgcccctacctggggtgccagagctggacatgcccctacacaggggccgtgagcagcccctactggaagttccagcaggatgtgaacttcacacaggaggtgagcaggctcaacatcaacctgcacttcagcaagtgcgggttcccattcagcctgctggtggatgcccccgggtacgatcccatctggtttctgaacacagaaccatcccagctgccccccacagccccccccctgctgccccacagcaacctggatcacatcttggagcccagcatcccctggaagagcaagttgctgacactggtgcagctgacattgcagagcacaaactacacatgcatcgtgtgcatcgatagggccagcctgagcacatggcacgtgttgtacagccccaacgtgagcgtgcccagctcaagtagcacacctctgctgtaccccagcctggccctgcccgccccccacctgacactgccattcaactggacacactgcttcgatccccagatccaggccattgtgagcagtccctgccacaacagtctgatcctgcccccatttagcctgagccccgtccccacactgggaagtaggagtaggagagctgtgccagtggccgtgtggctggtgagtgccctggctatgggcgctggggtcgccggaggaatcacagggagcatgagcctggccagcgggaagagcctgctgcacgaggtggataaggatatcagccagctgacacaggccatcgtgaagaaccacaagaacctgctgaagatcgcccagtacgccgcccagaacaggagggggctggacctgttgttctgggagaggggagggctgtgcaagtttctccaggagcagtgctgcttcctgaacatcacaaacagccacgtgagcattcttcaggagaggccccccctggagaacagggtgctgacagggtgggggctgaactgggatctgggactgagccagtgggccagggaggccttgcagacagggatcacactggtcgccctgttgctgctggtgatcctggccgggccctgcatcctgaggcagctgaggcacctgcccagtagggtgaggtacccccactacagcctgatcaaccccgagagcagcctgtgatgagcgcgcagcgctgacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 43 - DNA, 21391 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgggccaccatgggggtgaggaacagcgtgctgagcgggaagaaggccgatgagctggagaagatcaggctgaggcccaacgggaagaagaagtacatgctgaagcacgtggtgtgggccgccaacgagctggataggttcgggctggccgagagcctgctggagaacaaggaggggtgccagaagatcctgagcgtgctggcccccctggtgcccacagggagcgagaacctgaagagcctgtacaacacagtgtgcgtgatctggtgcatccacgccgaggagaaggtgaagcacacagaggaggccaagcagatcgtgcagaggcacctggtggtggagacagggacaacagagacaatgcccaagacaagcaggcccacagccccctccagcgggaggggcgggaactaccccgtgcagcagatcgggggcaactacgtgcacctgcccctgagccccaggacactgaacgcctgggtgaagctgatcgaggagaagaagttcggggccgaggtggtgcccgggttccaggccctgagcgaggggtgcacaccctacgatatcaaccagatgctgaactgcgtgggggatcaccaggccgccatgcagatcatcagggatatcatcaacgaggaggccgccgattgggatctgcaacacccacagcccgctccccaacaggggcagctgagggagcccagcgggagcgatatcgccgggacaacaagcagcgtggatgagcagatccagtggatgtacaggcagcagaaccccatccccgtcgggaacatctacaggaggtggatccagctggggctgcagaagtgcgtgaggatgtacaaccccacaaacatcctggatgtgaagcaggggccaaaggagccattccagagctacgtcgataggttctacaagagcctgagggccgagcagacagatgccgccgtgaagaactggatgacacagacactgctgatccagaacgccaaccccgattgcaagctggtgctgaaaggcctgggggtgaatcccacactggaggagatgttgacagcctgccagggggtggggggacccgggcagaaagccaggctgatggccgaagccctgaaggaagctctggctcccgtgcccatcccattcgccgctgcccagcagagggggcccaggaagcccatcaagtgctggaactgcgggaaggaggggcacagcgccaggcagtgcagggcccccaggaggcaggggtgctggaagtgcgggaagatggatcacgtgatggccaagtgccccgataggcaggccgggttcctggggctggggccctggggcaagaagcccaggaactttccaatggcccaggtgcaccaggggctgatgcccacagctccccccgaggatcccgccgtggatctgctgaagaactacatgcagctcgggaagcaacagagggagaagcagagggagagcagggagaagccctacaaggaggtgacagaggatttgctgcacctgaacagcctgttcgggggggatcagtgatagcgttcgaactacagctcaacttacctgccaaccccatgccagtcgacccaactagtacaacctaaatccattataaaaaacttaggaaccaggtccacacagccgccagcccatcaaccatccactcccacgattggaggccggcccatcgtacgcatgccaccatgagagtgaagggaatcagaaagaactaccagcacctgtggagatggggaaccatgctgctgggcatgctgatgatctgcagcgccgccgagcaggccagacagctgctgagcggcatcgtgcagcagcagaacaacctgctccgcgcaatcgaagctcaacagcacctgctgcaactgacagtgtggggaatcaagcagctgcaagcaagagtgctggccgtggaaagatacctgaaggaccaacagctcggcggcggcagcccctggaacgcttcatggagcaacaaaagcctggaccagatctggaacaacatgacctggatggagtgggagagagagatagacaactacacaagcctgatttacaccctgattgaagaaagccaaaatcaacaggagaaaaacgaacaagaactcctggagctggacaagtgggccagcctgtggaactggtttgacattaccaactggctgtggtacatcaagatcttcataatgatcgtcgggggcctcgtgggcctgagaatcgtgttcgccgtgctcagcatcgtgaatagagtgcggcagggctacagccccctgagctttcagtaataattcgaacatgcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 44 - DNA, 21391 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgggccaccatgggggtgaggaacagcgtgctgagcgggaagaaggccgatgagctggagaagatcaggctgaggcccaacgggaagaagaagtacatgctgaagcacgtggtgtgggccgccaacgagctggataggttcgggctggccgagagcctgctggagaacaaggaggggtgccagaagatcctgagcgtgctggcccccctggtgcccacagggagcgagaacctgaagagcctgtacaacacagtgtgcgtgatctggtgcatccacgccgaggagaaggtgaagcacacagaggaggccaagcagatcgtgcagaggcacctggtggtggagacagggacaacagagacaatgcccaagacaagcaggcccacagccccctccagcgggaggggcgggaactaccccgtgcagcagatcgggggcaactacgtgcacctgcccctgagccccaggacactgaacgcctgggtgaagctgatcgaggagaagaagttcggggccgaggtggtgcccgggttccaggccctgagcgaggggtgcacaccctacgatatcaaccagatgctgaactgcgtgggggatcaccaggccgccatgcagatcatcagggatatcatcaacgaggaggccgccgattgggatctgcaacacccacagcccgctccccaacaggggcagctgagggagcccagcgggagcgatatcgccgggacaacaagcagcgtggatgagcagatccagtggatgtacaggcagcagaaccccatccccgtcgggaacatctacaggaggtggatccagctggggctgcagaagtgcgtgaggatgtacaaccccacaaacatcctggatgtgaagcaggggccaaaggagccattccagagctacgtcgataggttctacaagagcctgagggccgagcagacagatgccgccgtgaagaactggatgacacagacactgctgatccagaacgccaaccccgattgcaagctggtgctgaaaggcctgggggtgaatcccacactggaggagatgttgacagcctgccagggggtggggggacccgggcagaaagccaggctgatggccgaagccctgaaggaagctctggctcccgtgcccatcccattcgccgctgcccagcagagggggcccaggaagcccatcaagtgctggaactgcgggaaggaggggcacagcgccaggcagtgcagggcccccaggaggcaggggtgctggaagtgcgggaagatggatcacgtgatggccaagtgccccgataggcaggccgggttcctggggctggggccctggggcaagaagcccaggaactttccaatggcccaggtgcaccaggggctgatgcccacagctccccccgaggatcccgccgtggatctgctgaagaactacatgcagctcgggaagcaacagagggagaagcagagggagagcagggagaagccctacaaggaggtgacagaggatttgctgcacctgaacagcctgttcgggggggatcagtgatagcgttcgaactacagctcaacttacctgccaaccccatgccagtcgacccaactagtacaacctaaatccattataaaaaacttaggaaccaggtccacacagccgccagcccatcaaccatccactcccacgattggaggccggcccatcgtacgcatgccaccatgagagtgaagggaatcagaaagaactaccagcacctgtggagatggggaaccatgctgctgggcatgctgatgatctgcagcgccgccgagcaggccagacagctgctgagcggcatcgtgcagcagcagaacaacctgctccgcgcaatcgaagctcaacagcacctgctgcaactgacagtgtggggaatcaagcagctgcaagcaagagtgctggccgtggaaagaagactgaaggaccaacagctcggcggcggcagcccctggaacgcttcatggagcaacaaaagcctggaccagatctggaacaacatgacctggatggagtgggagagagagatagacaactacacaagcctgatttacaccctgattgaagaaagccaaaatcaacaggagaaaaacgaacaagaactcctggagctggacaagtgggccagcctgtggaactggtttgacattaccaactggctgtggtacatcaagatcttcataatgatcgtcgggggcctcgtgggcctgagaatcgtgttcgccgtgctcagcatcgtgaatagagtgcggcagggctacagccccctgagctttcagtaataattcgaacatgcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 45 - protein, 429 aa, Artificial Sequence
  Synthetic sequence
MGQIFSRSASPIPRPPRGLAAHHWLNFLQAAYRLEPGPSSYDFHQLKKFLKIALETPARICPINYSLLASLLPKGYPGRVNEILHILIQTQAQIPSRPAPPPPSSPTHDPPDSDPQIPPPYVEPTAPQVLPVMHPHGAPPNHRPWQMKDLQAIKQEVSQAAPGSPQFMQTIRLAVQQFDPTAKDLQDLLQYLCSSLVASLHHQQLDSLISEAETRGITGYNPLAGPLRVQANNPQQQGLRREYQQLWLAAFAALPGSAKDPSWASILQGLEEPYHAFVERLNIALDNGLPEGTPKDPILRSLAYSNANKECQKLLQARGHTNSPLGDMLRACQTWTPKDKTKVLVVQPKKPPPNQPCFRCGKAGHWSRDCTQPRPPPGPCPLCQDPTHWKRDCPRLKPTIPEPEPEEDALLLDLPADIPHPKNSIGGEV

SEQ ID NO: 46 - protein, 1462 aa, Artificial Sequence
  Synthetic sequence
MGQIFSRSASPIPRPPRGLAAHHWLNFLQAAYRLEPGPSSYDFHQLKKFLKIALETPARICPINYSLLASLLPKGYPGRVNEILHILIQTQAQIPSRPAPPPPSSPTHDPPDSDPQIPPPYVEPTAPQVLPVMHPHGAPPNHRPWQMKDLQAIKQEVSQAAPGSPQFMQTIRLAVQQFDPTAKDLQDLLQYLCSSLVASLHHQQLDSLISEAETRGITGYNPLAGPLRVQANNPQQQGLRREYQQLWLAAFAALPGSAKDPSWASILQGLEEPYHAFVERLNIALDNGLPEGTPKDPILRSLAYSNANKECQKLLQARGHTNSPLGDMLRACQTWTPKDKTKVLVVQPKKPPPNQPCFRCGKAGHWSRDCTQPRPPPGPCPLCQDPTHWKRDCPRLKPTIPEPEPEEDALLLDLPADIPHPKNLHRGGGLTSPPTLQQVLPNQDPASILPVIPLDPARRPVIKAQVDTQTSHPKTIEALLDTGADMTVLPIALFSSNTPLKNTSVLGAGGQTQDHFKLTSLPVLIRLPFRTTPIVLTSCLVDTKNNWAIIGRDALQQCQGVLYLPEAKRPPVILPIQAPAVLGLEHLPRPPQISQFPLNPERLQALQHLVRKALEAGHIEPYTGPGNNPVFPVKKANGTWRFIHDLRATNSLTIDLSSSSPGPPDLSSLPTTLAHLQTIDLRDAFFQIPLPKQFQPYFAFTVPQQCNYGPGTRYAWKVLPQGFKNSPTLFEMQLAHILQPIRQAFPQCTILQYMDDILLASPSHEDLLLLSEATMASLISHGLPVSENKTQQTPGTIKFLGQIISPNHLTYDAVPTVPIRSRWALPELQALLGEIQWVSKGTPTLRQPLHSLYCALQRHTDPRDQIYLNPSQVQSLVQLRQALSQNCRSRLVQTLPLLGAIMLTLTGTTTVVFQSKEQWPLVWLHAPLPHTSQCPWGQLLASAVLLLDKYTLQSYGLLCQTIHHNISTQTFNQFIQTSDHPSVPILLHHSHRFKNLGAQTGELWNTFLKTAAPLAPVKALMPVFTLSPVIINTAPCLFSDGSTSRAAYILWDKQILSQRSFPLPPPHKSAQRAELLGLLHGLSSARSWRCLNIFLDSKYLYHYLRTLALGTFQGRSSQAPFQALLPRLLSRKVVYLHHVRSHTNLPDPISRLNALTDALLITPVLQLSPAELHSFTHCGQTALTLQGATTTEASNILRSCHACRGGNPQHQMPRGHIRRGLLPNHIWQGDITHFKYKNTLYRLHVWVDTFSGAISATQKRKETSSEAISSLLQAIAHLGKPSYINTDNGPAYISQDFLNMCTSLAIRHTTHVPYNPTSSGLVERSNGILKTLLYKYFTDKPDLPMDNALSIALWTINHLNVLTNCHKTRWQLHHSPRLQPIPETRSLSNKQTHWYYFKLPGLNSRQWKGPQEALQEAAGAALIPVSASSAQWIPWRLLKRAACPRPVGGPADPKEKDLQHHG

SEQ ID NO: 47 - protein, 488 aa, Artificial Sequence
  Synthetic sequence
MGKFLATLILFFQFCPLILGDYSPSCCTLTIGVSSYHSKPCNPAQPVCSWTLDLLALSADQALQPPCPNLVSYSSYHATYSLYLFPHWIKKPNRNGGGYYSASYSDPCSLKCPYLGCQSWTCPYTGAVSSPYWKFQQDVNFTQEVSRLNINLHFSKCGFPFSLLVDAPGYDPIWFLNTEPSQLPPTAPPLLPHSNLDHILEPSIPWKSKLLTLVQLTLQSTNYTCIVCIDRASLSTWHVLYSPNVSVPSSSSTPLLYPSLALPAPHLTLPFNWTHCFDPQIQAIVSSPCHNSLILPPFSLSPVPTLGSRSRRAVPVAVWLVSALAMGAGVAGGITGSMSLASGKSLLHEVDKDISQLTQAIVKNHKNLLKIAQYAAQNRRGLDLLFWEQGGLCKALQEQCCFLNITNSHVSILQERPPLENRVLTGWGLNWDLGLSQWAREALQTGITLVALLLLVILAGPCILRQLRHLPSRVRYPHYSLINPESSL

SEQ ID NO: 48 - protein, 488 aa, Artificial Sequence
  Synthetic sequence
MGKFLATLILFFQFCPLILGDYSPSCCTLTIGVSSYHSKPCNPAQPVCSWTLDLLALSADQALQPPCPNLVSYSSYHATYSLYLFPHWIKKPNRNGGGYYSASYSDPCSLKCPYLGCQSWTCPYTGAVSSPYWKFQQDVNFTQEVSRLNINLHFSKCGFPFSLLVDAPGYDPIWFLNTEPSQLPPTAPPLLPHSNLDHILEPSIPWKSKLLTLVQLTLQSTNYTCIVCIDRASLSTWHVLYSPNVSVPSSSSTPLLYPSLALPAPHLTLPFNWTHCFDPQIQAIVSSPCHNSLILPPFSLSPVPTLGSRSRRAVPVAVWLVSALAMGAGVAGGITGSMSLASGKSLLHEVDKDISQLTQAIVKNHKNLLKIAQYAAQNRRGLDLLFWERGGLCKFLQEQCCFLNITNSHVSILQERPPLENRVLTGWGLNWDLGLSQWAREALQTGITLVALLLLVILAGPCILRQLRHLPSRVRYPHYSLINPESSL

SEQ ID NO: 49 - protein, 206 aa, Artificial Sequence
  Synthetic sequence
MAASGLFRCLPVSCPEDLLVEELVDGAASLEEELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQ

SEQ ID NO: 50 - protein, 353 aa, Artificial Sequence
  Synthetic sequence
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEELLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHSLHLLFEEYTNIPISLLFNEKEADDNDHEPQISPGGLEPPSEKHFRETEV

SEQ ID NO: 51 - protein, 317 aa, Artificial Sequence
  Synthetic sequence
METDTLLLWVLLLWVPGSTGAASGLFRCLPVSCPEDLLVEELVDGAASLEEELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQGGGGSGFGQSLLFGYPVYVFGDCVGGGGSLIQYSSFHSLHLLFEEYTNGGGGSGSPNKGSGTTSGTTRLLSGHTCFTLTGLLGTLVTMGLLT

SEQ ID NO: 52 - protein, 486 aa, Artificial Sequence
  Synthetic sequence
MGNVFFLLLFSLTHFPLAQQSRCTLTIGISSYHSSPCSPTQPVCTWNLDLNSLTTDQRLHPPCPNLITYSGFHKTYSLYLFPHWIKKPNRQGLGYYSPSYNDPCSLQCPYLGCQAWTSAYTGPVSSPSWKFHSDVNFTQEVSQVSLRLHFSKCGSSMTLLVDAPGYDPLWFITSEPTQPPPTSPPLVHDSDLEHVLTPSTSWTTKILKFIQLTLQSTNYSCMVCVDRSSLSSWHVLYTPNISIPQQTSSRTILFPSLALPAPPSQPFPWTHCYQPRLQAITTDNCNNSIILPPFSLAPVPPPATRRRRAVPIAVWLVSALAAGTGIAGGVTGSLSLASSKSLLLEVDKDISHLTQAIVKNHQNILRVAQYAAQNRRGLDLLFWEQGGLCKAIQEQCCFLNISNTHVSVLQERPPLEKRVITGWGLNWDLGLSQWAREALQTGITILALLLLVILFGPCILRQIQALPQRLQNRHNQYSLINPETML

SEQ ID NO: 53 - protein, 486 aa, Artificial Sequence
  Synthetic sequence
MGNVFFLLLFSLTHFPLAQQSRCTLTIGISSYHSSPCSPTQPVCTWNLDLNSLTTDQRLHPPCPNLITYSGFHKTYSLYLFPHWIKKPNRQGLGYYSPSYNDPCSLQCPYLGCQAWTSAYTGPVSSPSWKFHSDVNFTQEVSQVSLRLHFSKCGSSMTLLVDAPGYDPLWFITSEPTQPPPTSPPLVHDSDLEHVLTPSTSWTTKILKFIQLTLQSTNYSCMVCVDRSSLSSWHVLYTPNISIPQQTSSRTILFPSLALPAPPSQPFPWTHCYQPRLQAITTDNCNNSIILPPFSLAPVPPPATRRRRAVPIAVWLVSALAAGTGIAGGVTGSLSLASSKSLLLEVDKDISHLTQAIVKNHQNILRVAQYAAQNRRGLDLLFWERGGLCKFIQEQCCFLNISNTHVSVLQERPPLEKRVITGWGLNWDLGLSQWAREALQTGITILALLLLVILFGPCILRQIQALPQRLQNRHNQYSLINPETML

SEQ ID NO: 54 - DNA, 21959 bp, Artificial Sequence
  Synthetic sequence
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccgccaccatggggcagatctttagcaggagtgctagcccaattcccagaccacccagggggctggccgcccaccactggctgaactttctgcaggccgcctacaggctggagcccgggcccagcagctacgatttccaccagctgaagaagttcctgaagatcgccctggagacacccgtgtggatctgcccaatcaactacagcctgctggccagcctgctgcccaaaggctaccccgggagggtgaacgagatcctgcacatcctgatccagacacaggctcagatcccaagtaggccagccccaccaccccccagcagccccacacacgatccccccgatagcgatccccagatcccccccccatacgtggagcccacagccccacaggtgctgccagtgatgcatccccacggggccccccccaaccacaggccctggcagatgaaggacctccaggccatcaagcaggaggtgagccaggccgcccccgggagcccccagttcatgcagacaatcaggctggccgtgcagcagttcgatcccacagccaaggatctgcaggatctgctgcagtacctgtgcagcagcctggtggcctccctgcaccaccagcagctggatagcctgatcagcgaggccgagaccagggggatcacagggtacaaccccctggccgggcccctgagggtgcaggccaacaacccacagcagcaggggctgaggagagagtatcagcaactgtggttggctgccttcgccgccctgcccgggagcgccaaggatccaagctgggccagcattctgcaggggctggaggagccctaccacgccttcgtggagaggctgaacatcgccctggataacgggctgcccgaggggacaccaaaggatcccatcctgaggagcctggcctacagcaacgccaacaaggagtgccagaagctgctgcaggccagggggcacacaaacagcccactgggggatatgctgagggcctgccagacatggacacccaaggataagacaaaggtgctggtcgtgcagcccaagaagcccccacccaaccagccctgcttcaggtgcgggaaggccgggcattggagtagagattgtacacaaccaaggccaccacccgggccctgccccctgtgccaggatcccacacactggaagagggattgccccaggctgaagcccacaatccccgagccagagcccgaggaggatgccctgctgctggatctgcccgccgatatcccccaccccaagaacagcatcgggggggaggtgtgatgagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacggccaccatgggcaagttcctggccacactgatcctgttcttccagttctgccccctcatcctgggggattacagcccaagctgctgcacactgacaatcggggtgagcagctaccacagcaaaccctgcaaccccgcccagcccgtgtgcagctggacactggatctgctggccctgagcgccgatcaggctctgcagcccccctgccccaacctggtgagctacagctcctaccacgccacatacagcctgtacctgttccctcactggatcaagaagcccaacaggaacgggggggggtactacagcgccagctacagcgatccctgcagcctgaagtgcccctacctggggtgccagagctggacatgcccctacacaggggccgtgagcagcccctactggaagttccagcaggatgtgaacttcacacaggaggtgagcaggctcaacatcaacctgcacttcagcaagtgcgggttcccattcagcctgctggtggatgcccccgggtacgatcccatctggtttctgaacacagaaccatcccagctgccccccacagccccccccctgctgccccacagcaacctggatcacatcttggagcccagcatcccctggaagagcaagttgctgacactggtgcagctgacattgcagagcacaaactacacatgcatcgtgtgcatcgatagggccagcctgagcacatggcacgtgttgtacagccccaacgtgagcgtgcccagctcaagtagcacacctctgctgtaccccagcctggccctgcccgccccccacctgacactgccattcaactggacacactgcttcgatccccagatccaggccattgtgagcagtccctgccacaacagtctgatcctgcccccatttagcctgagccccgtccccacactgggaagtaggagtaggagagctgtgccagtggccgtgtggctggtgagtgccctggctatgggcgctggggtcgccggaggaatcacagggagcatgagcctggccagcgggaagagcctgctgcacgaggtggataaggatatcagccagctgacacaggccatcgtgaagaaccacaagaacctgctgaagatcgcccagtacgccgcccagaacaggagggggctggacctgttgttctgggagcagggagggctgtgcaaggccctccaggagcagtgctgcttcctgaacatcacaaacagccacgtgagcattcttcaggagaggccccccctggagaacagggtgctgacagggtgggggctgaactgggatctgggactgagccagtgggccagggaggccttgcagacagggatcacactggtcgccctgttgctgctggtgatcctggccgggccctgcatcctgaggcagctgaggcacctgcccagtagggtgaggtacccccactacagcctgatcaaccccgagagcagcctgtgatgagcgcgcagcgctgacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 55 - protein, 206 aa, Artificial Sequence
  Synthetic sequence
MAASGLFRCLPVSCPEDLLVEELVDGLLSLEEELKDKEEEKAVLDGLLSLEEESRGRLRRGPPGEKAPPRGETHRDRQRRAEEKRKRKKEREKEEEKQIAEYLKRKEEEKARRRRRAEKKAADVARRKQEEQERRERKWRQGAEKAKQHSARKEKMQELGIDGYTRQLEGEVESLEAERRKLLQEKEDLMGEVNYWQGRLEAMWLQ